SEQUENCE LISTING (85 sequences)

SEQ ID NO: 1 - protein, 8 aa, synthetic construct
DHTIHWMR

SEQ ID NO: 2 - protein, 18 aa, synthetic construct
YIYPRDGITGYNERFRGK

SEQ ID NO: 3 - protein, 20 aa, synthetic construct
WGYSYRNYAYYYDYWGQGTL

SEQ ID NO: 4 - protein, 16 aa, synthetic construct
INCRSSQSLLHSSNQR

SEQ ID NO: 5 - protein, 14 aa, synthetic construct
PPKVLIYWASTRES

SEQ ID NO: 6 - protein, 12 aa, synthetic construct
QQYYSYPWTFGQ

SEQ ID NO: 7 - protein, 123 aa, synthetic construct
QVQLVQSGAEVKKPGASVKVSCKASGYTLTDHTIHWMRQAPGQRLEWIGYIYPRDGITGYNERFRGKATLTADTSASTAYMELSSLRSEDTAVYYCARWGYSYRNYAYYYDYWGQGTLVTVSS

SEQ ID NO: 8 - protein, 113 aa, synthetic construct
DIVMTQSPDSLAVSLGERATINCRSSQSLLHSSNQRNYLAWYQQKPGQPPKVLIYWASTRESGVPDRFSGSGSGTDFTLTISSLQAEDVAVYYCQQYYSYPWTFGQGTKVEIK

SEQ ID NO: 9 - protein, 274 aa, synthetic construct
MEAPAQLLFLLLLWLPDTTGDIVMTQSPDSLAVSLGERATINCRSSQSLLHSSNQRNYLAWYQQKPGQPPKVLIYWASTRESGVPDRFSGSGSGTDFTLTISSLQAEDVAVYYCQQYYSYPWTFGQGTKVEIKGGGGSGGGGSGGGGSQVQLVQSGAEVKKPGASVKVSCKASGYTLTDHTIHWMRQAPGQRLEWIGYIYPRDGITGYNERFRGKATLTADTSASTAYMELSSLRSEDTAVYYCARWGYSYRNYAYYYDYWGQGTLVTVSSSPV

SEQ ID NO: 10 - protein, 15 aa, synthetic construct
EPKSCDKTHTCPPCP

SEQ ID NO: 11 - protein, 140 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEE

SEQ ID NO: 12 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 13 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 473 aa, synthetic construct
MEAPAQLLFLLLLWLPDTTGDIVMTQSPDSLAVSLGERATINCRSSQSLLHSSNQRNYLAWYQQKPGQPPKVLIYWASTRESGVPDRFSGSGSGTDFTLTISSLQAEDVAVYYCQQYYSYPWTFGQGTKVEIKGGGGSGGGGSGGGGSQVQLVQSGAEVKKPGASVKVSCKASGYTLTDHTIHWMRQAPGQRLEWIGYIYPRDGITGYNERFRGKATLTADTSASTAYMELSSLRSEDTAVYYCARWGYSYRNYAYYYDYWGQGTLVTVSSSPVEPKSCDKTHTCPPCPPLIKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 598 aa, synthetic construct
MEAPAQLLFLLLLWLPDTTGDIVMTQSPDSLAVSLGERATINCRSSQSLLHSSNQRNYLAWYQQKPGQPPKVLIYWASTRESGVPDRFSGSGSGTDFTLTISSLQAEDVAVYYCQQYYSYPWTFGQGTKVEIKGGGGSGGGGSGGGGSQVQLVQSGAEVKKPGASVKVSCKASGYTLTDHTIHWMRQAPGQRLEWIGYIYPRDGITGYNERFRGKATLTADTSASTAYMELSSLRSEDTAVYYCARWGYSYRNYAYYYDYWGQGTLVTVSSSPVKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEEPLIKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 815 bp, synthetic construct
  source 1..815: mol_type = other DNA
atggaggcccccgcccagctgctgttcctgctgctgctgtggctgcccgacaccaccggcgacatcgtgatgacccagagccccgacagcctggccgtgagcctgggcgagagagccaccatcaactgcagaagcagccagagcctgctgcacagcagcaaccagagaaactacctggcctggtaccagcagaagcccggccagccccccaaggtgctgatctactgggccagcaccagagagagcggcgtgcccgacagattcagcggcagcggcagcggcaccgacttcaccctgaccatcagcagcctgcaggccgaggacgtggccgtgtactactgccagcagtactacagctacccctggaccttcggccagggcaccaaggtggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccctgaccgaccacaccatccactggatgagacaggcccccggccagagactggagtggatcggctacatctaccccagagacggcatcaccggctacaacgagagattcagaggcaaggccaccctgaccgccgacaccagcgccagcaccgcctacatggagctgagcagcctgagaagcgaggacaccgccgtgtactactgcgccagatggggctacagctacagaaactacgcctactactacgactactggggccagggcaccctggtgaccgtgagcagctc

SEQ ID NO: 17 - DNA, 1422 bp, synthetic construct
  source 1..1422: mol_type = other DNA
atggaggcccccgcccagctgctgttcctgctgctgctgtggctgcccgacaccaccggcgacatcgtgatgacccagagccccgacagcctggccgtgagcctgggcgagagagccaccatcaactgcagaagcagccagagcctgctgcacagcagcaaccagagaaactacctggcctggtaccagcagaagcccggccagccccccaaggtgctgatctactgggccagcaccagagagagcggcgtgcccgacagattcagcggcagcggcagcggcaccgacttcaccctgaccatcagcagcctgcaggccgaggacgtggccgtgtactactgccagcagtactacagctacccctggaccttcggccagggcaccaaggtggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccctgaccgaccacaccatccactggatgagacaggcccccggccagagactggagtggatcggctacatctaccccagagacggcatcaccggctacaacgagagattcagaggcaaggccaccctgaccgccgacaccagcgccagcaccgcctacatggagctgagcagcctgagaagcgaggacaccgccgtgtactactgcgccagatggggctacagctacagaaactacgcctactactacgactactggggccagggcaccctggtgaccgtgagcagctcaccggtcgagcccaagagctgcgacaagacccacacctgtcccccctgcccccccttaattaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 18 - DNA, 1797 bp, synthetic construct
  source 1..1797: mol_type = other DNA
atggaggcccccgcccagctgctgttcctgctgctgctgtggctgcccgacaccaccggcgacatcgtgatgacccagagccccgacagcctggccgtgagcctgggcgagagagccaccatcaactgcagaagcagccagagcctgctgcacagcagcaaccagagaaactacctggcctggtaccagcagaagcccggccagccccccaaggtgctgatctactgggccagcaccagagagagcggcgtgcccgacagattcagcggcagcggcagcggcaccgacttcaccctgaccatcagcagcctgcaggccgaggacgtggccgtgtactactgccagcagtactacagctacccctggaccttcggccagggcaccaaggtggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccctgaccgaccacaccatccactggatgagacaggcccccggccagagactggagtggatcggctacatctaccccagagacggcatcaccggctacaacgagagattcagaggcaaggccaccctgaccgccgacaccagcgccagcaccgcctacatggagctgagcagcctgagaagcgaggacaccgccgtgtactactgcgccagatggggctacagctacagaaactacgcctactactacgactactggggccagggcaccctggtgaccgtgagcagctcaccggtcaaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaggacaagcagaacaccgtgtgtgaagagtgccccgacggcacctacagcgacgaggccgcccgggccgccgacgccgagtgcgaggaacccttaattaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 19 - DNA, 45 bp, synthetic construct
  source 1..45: mol_type = other DNA
gagcccaagagctgcgacaagacccacacctgtcccccctgcccc

SEQ ID NO: 20 - DNA, 420 bp, synthetic construct
  source 1..420: mol_type = other DNA
aaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaggacaagcagaacaccgtgtgtgaagagtgccccgacggcacctacagcgacgaggccgcccgggccgccgacgccgagtgcgaggaa

SEQ ID NO: 21 - skipped

SEQ ID NO: 22 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 23 - protein, 680 aa, synthetic construct
MEAPAQLLFLLLLWLPDTTGDIVMTQSPDSLAVSLGERATINCRSSQSLLHSSNQRNYLAWYQQKPGQPPKVLIYWASTRESGVPDRFSGSGSGTDFTLTISSLQAEDVAVYYCQQYYSYPWTFGQGTKVEIKGGGGSGGGGSGGGGSQVQLVQSGAEVKKPGASVKVSCKASGYTLTDHTIHWMRQAPGQRLEWIGYIYPRDGITGYNERFRGKATLTADTSASTAYMELSSLRSEDTAVYYCARWGYSYRNYAYYYDYWGQGTLVTVSSSPVKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNPLIKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 689 aa, synthetic construct
MEAPAQLLFLLLLWLPDTTGDIVMTQSPDSLAVSLGERATINCRSSQSLLHSSNQRNYLAWYQQKPGQPPKVLIYWASTRESGVPDRFSGSGSGTDFTLTISSLQAEDVAVYYCQQYYSYPWTFGQGTKVEIKGGGGSGGGGSGGGGSQVQLVQSGAEVKKPGASVKVSCKASGYTLTDHTIHWMRQAPGQRLEWIGYIYPRDGITGYNERFRGKATLTADTSASTAYMELSSLRSEDTAVYYCARWGYSYRNYAYYYDYWGQGTLVTVSSSPVEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPLIKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 2043 bp, synthetic construct
  source 1..2043: mol_type = other DNA
atggaggcccccgcccagctgctgttcctgctgctgctgtggctgcccgacaccaccggcgacatcgtgatgacccagagccccgacagcctggccgtgagcctgggcgagagagccaccatcaactgcagaagcagccagagcctgctgcacagcagcaaccagagaaactacctggcctggtaccagcagaagcccggccagccccccaaggtgctgatctactgggccagcaccagagagagcggcgtgcccgacagattcagcggcagcggcagcggcaccgacttcaccctgaccatcagcagcctgcaggccgaggacgtggccgtgtactactgccagcagtactacagctacccctggaccttcggccagggcaccaaggtggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccctgaccgaccacaccatccactggatgagacaggcccccggccagagactggagtggatcggctacatctaccccagagacggcatcaccggctacaacgagagattcagaggcaaggccaccctgaccgccgacaccagcgccagcaccgcctacatggagctgagcagcctgagaagcgaggacaccgccgtgtactactgcgccagatggggctacagctacagaaactacgcctactactacgactactggggccagggcaccctggtgaccgtgagcagctcaccggtcaaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaagacaagcagaataccgtgtgtgaagagtgccccgacggcacctacagcgacgaggccaaccacgtggacccctgcctgccctgcactgtgtgcgaggacaccgagcggcagctgcgcgagtgcacaagatgggccgacgccgagtgcgaagagatccccggcagatggatcaccagaagcaccccccctgagggcagcgacagcaccgcccctagcacccaggaacctgaggcccctcccgagcaggacctgatcgcctctacagtggccggcgtggtgacaaccgtgatgggcagctctcagcccgtggtgacacggggcaccaccgacaatcccttaattaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 26 - DNA, 2070 bp, synthetic construct
  source 1..2070: mol_type = other DNA
atggaggcccccgcccagctgctgttcctgctgctgctgtggctgcccgacaccaccggcgacatcgtgatgacccagagccccgacagcctggccgtgagcctgggcgagagagccaccatcaactgcagaagcagccagagcctgctgcacagcagcaaccagagaaactacctggcctggtaccagcagaagcccggccagccccccaaggtgctgatctactgggccagcaccagagagagcggcgtgcccgacagattcagcggcagcggcagcggcaccgacttcaccctgaccatcagcagcctgcaggccgaggacgtggccgtgtactactgccagcagtactacagctacccctggaccttcggccagggcaccaaggtggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccctgaccgaccacaccatccactggatgagacaggcccccggccagagactggagtggatcggctacatctaccccagagacggcatcaccggctacaacgagagattcagaggcaaggccaccctgaccgccgacaccagcgccagcaccgcctacatggagctgagcagcctgagaagcgaggacaccgccgtgtactactgcgccagatggggctacagctacagaaactacgcctactactacgactactggggccagggcaccctggtgaccgtgagcagctcaccggtcgagcccaagagccccgacaagacccacacctgtcccccctgtcctgcccctccagtggccggacctagcgtgttcctgttccccccaaagcccaaggacaccctgatgatcgcccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagaatacaagtgcaaggtctccaacaaggccctgcctgcccccatcgagaaaaccatcagcaaggccaagggccagccccgcgagccccaggtgtacacactgccccccagccgggacgagctgaccaagaaccaggtgtccctgacctgcctcgtgaaaggcttctaccccagcgatatcgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcaagcccttaattaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 27 - protein, 222 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDN

SEQ ID NO: 28 - protein, 231 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 666 bp, synthetic construct
  source 1..666: mol_type = other DNA
aaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaagacaagcagaataccgtgtgtgaagagtgccccgacggcacctacagcgacgaggccaaccacgtggacccctgcctgccctgcactgtgtgcgaggacaccgagcggcagctgcgcgagtgcacaagatgggccgacgccgagtgcgaagagatccccggcagatggatcaccagaagcaccccccctgagggcagcgacagcaccgcccctagcacccaggaacctgaggcccctcccgagcaggacctgatcgcctctacagtggccggcgtggtgacaaccgtgatgggcagctctcagcccgtggtgacacggggcaccaccgacaat

SEQ ID NO: 30 - DNA, 693 bp, synthetic construct
  source 1..693: mol_type = other DNA
gagcccaagagccccgacaagacccacacctgtcccccctgtcctgcccctccagtggccggacctagcgtgttcctgttccccccaaagcccaaggacaccctgatgatcgcccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagaatacaagtgcaaggtctccaacaaggccctgcctgcccccatcgagaaaaccatcagcaaggccaagggccagccccgcgagccccaggtgtacacactgccccccagccgggacgagctgaccaagaaccaggtgtccctgacctgcctcgtgaaaggcttctaccccagcgatatcgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcaag

SEQ ID NO: 31 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 32 - protein, 162 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEE

SEQ ID NO: 33 - protein, 200 aa, synthetic construct
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEEIPGRWITRSTPPEGSDSTAPSTQEPEAPPEQDLIASTVAGVVTTVMGSSQPVVTRGTTDN

SEQ ID NO: 34 - protein, 34 aa, synthetic construct
ACPTGLYTHSGECCKACNLGEGVAQPCGANQTVC

SEQ ID NO: 35 - protein, 42 aa, synthetic construct
PCLDSVTFSDVVSATEPCKPCTECVGLQSMSAPCVEADDAVC

SEQ ID NO: 36 - protein, 39 aa, synthetic construct
RCAYGYYQDETTGRCEACRVCEAGSGLVFSCQDKQNTVC

SEQ ID NO: 37 - protein, 41 aa, synthetic construct
ECPDGTYSDEANHVDPCLPCTVCEDTERQLRECTRWADAEC

SEQ ID NO: 38 - protein, 25 aa, synthetic construct
NHVDPCLPCTVCEDTERQLRECTRW

SEQ ID NO: 39 - protein, 16 aa, synthetic construct
RSSQSIVHSNGNTYLE

SEQ ID NO: 40 - protein, 7 aa, synthetic construct
KVSNRFS

SEQ ID NO: 41 - protein, 9 aa, synthetic construct
FQGSHVPLT

SEQ ID NO: 42 - protein, 5 aa, synthetic construct
DYYMY

SEQ ID NO: 43 - protein, 17 aa, synthetic construct
SISFDGTYTYYTDRVKG

SEQ ID NO: 44 - protein, 8 aa, synthetic construct
DRPAWFPY

SEQ ID NO: 45 - protein, 268 aa, synthetic construct
MEAPAQLLFLLLLWLPDTTGQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKGAP

SEQ ID NO: 46 - DNA, 804 bp, synthetic construct
  source 1..804: mol_type = other DNA
atggaggcccccgcccagctgctgttcctgctgctgctgtggctgcccgacaccaccggccaggtgcagctggtggagagcggcggcggcgtggtgcagcccggcagaagcctgagactgagctgcgccgccagcggcttcaccttcagcgactactacatgtactgggtgagacaggcccccggcaagggcctggagtgggtggccagcatcagcttcgacggcacctacacctactacaccgacagagtgaagggcagattcaccatcagcagagacaacagcaagaacaccctgtacctgcagatgaacagcctgagagccgaggacaccgccgtgtactactgcgccagagacagacccgcctggttcccctactggggccagggcaccctggtgaccgtgagcgccggcggcggcggcagcggcggcggcggcagcggcggcggcggcagcggcgacatcgtgatgacccagacccccctgagcctgagcgtgacccccggccagcccgccagcatcagctgcagaagcagccagagcatcgtgcacagcaacggcaacacctacctggagtggtacctgcagaagcccggccagagcccccagctgctgatctacaaggtgagcaacagattcagcggcgtgcccgacagattcagcggcagcggcagcggcaccgacttcaccctgaagatcagcagagtggaggccgaggacgtgggcgtgtactactgcttccagggcagccacgtgcccctgaccttcggcgccggcaccaagctggagctgaagggcgccccc

SEQ ID NO: 47 - protein, 516 aa, synthetic construct
MEAPAQLLFLLLLWLPDTTGDIVMTQSPDSLAVSLGERATINCRSSQSLLHSSNQRNYLAWYQQKPGQPPKVLIYWASTRESGVPDRFSGSGSGTDFTLTISSLQAEDVAVYYCQQYYSYPWTFGQGTKVEIKGGGGSGGGGSGGGGSQVQLVQSGAEVKKPGASVKVSCKASGYTLTDHTIHWMRQAPGQRLEWIGYIYPRDGITGYNERFRGKATLTADTSASTAYMELSSLRSEDTAVYYCARWGYSYRNYAYYYDYWGQGTLVTVSSAAATSSGESKYGPPCPPCPDIFWVLVVVGGVLACYSLLVTVAFIIFWVRSKRSRLLHSDYMNMTPRRPGPTRKHYQPYAPPRDFAAYRSASKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 595 aa, synthetic construct
MEAPAQLLFLLLLWLPDTTGQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKGAPSPVKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEEPLIKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 677 aa, synthetic construct
MEAPAQLLFLLLLWLPDTTGQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKGAPSPVKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNPLIKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 470 aa, synthetic construct
MEAPAQLLFLLLLWLPDTTGQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKGAPSPVEPKSCDKTHTCPPCPPLIKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - DNA, 1551 bp, synthetic construct
  source 1..1551: mol_type = other DNA
atggaggcccccgcccagctgctgttcctgctgctgctgtggctgcccgacaccaccggcgacatcgtgatgacccagagccccgacagcctggccgtgagcctgggcgagagagccaccatcaactgcagaagcagccagagcctgctgcacagcagcaaccagagaaactacctggcctggtaccagcagaagcccggccagccccccaaggtgctgatctactgggccagcaccagagagagcggcgtgcccgacagattcagcggcagcggcagcggcaccgacttcaccctgaccatcagcagcctgcaggccgaggacgtggccgtgtactactgccagcagtactacagctacccctggaccttcggccagggcaccaaggtggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccctgaccgaccacaccatccactggatgagacaggcccccggccagagactggagtggatcggctacatctaccccagagacggcatcaccggctacaacgagagattcagaggcaaggccaccctgaccgccgacaccagcgccagcaccgcctacatggagctgagcagcctgagaagcgaggacaccgccgtgtactactgcgccagatggggctacagctacagaaactacgcctactactacgactactggggccagggcaccctggtgaccgtgagcagcgccgccgccaccagcagcggcgagagcaagtacggccccccctgccccccctgccccgacatcttctgggtgctggtggtggtgggcggcgtgctggcctgctacagcctgctggtgaccgtggccttcatcatcttctgggtgagaagcaagagaagcagactgctgcacagcgactacatgaacatgacccccagaagacccggccccaccagaaagcactaccagccctacgccccccccagagacttcgccgcctacagaagcgccagcaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctacaagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagatga

SEQ ID NO: 52 - DNA, 1788 bp, synthetic construct
  source 1..1788: mol_type = other DNA
atggaggcccccgcccagctgctgttcctgctgctgctgtggctgcccgacaccaccggccaggtgcagctggtggagagcggcggcggcgtggtgcagcccggcagaagcctgagactgagctgcgccgccagcggcttcaccttcagcgactactacatgtactgggtgagacaggcccccggcaagggcctggagtgggtggccagcatcagcttcgacggcacctacacctactacaccgacagagtgaagggcagattcaccatcagcagagacaacagcaagaacaccctgtacctgcagatgaacagcctgagagccgaggacaccgccgtgtactactgcgccagagacagacccgcctggttcccctactggggccagggcaccctggtgaccgtgagcgccggcggcggcggcagcggcggcggcggcagcggcggcggcggcagcggcgacatcgtgatgacccagacccccctgagcctgagcgtgacccccggccagcccgccagcatcagctgcagaagcagccagagcatcgtgcacagcaacggcaacacctacctggagtggtacctgcagaagcccggccagagcccccagctgctgatctacaaggtgagcaacagattcagcggcgtgcccgacagattcagcggcagcggcagcggcaccgacttcaccctgaagatcagcagagtggaggccgaggacgtgggcgtgtactactgcttccagggcagccacgtgcccctgaccttcggcgccggcaccaagctggagctgaagggcgccccctcaccggtcaaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaggacaagcagaacaccgtgtgtgaagagtgccccgacggcacctacagcgacgaggccgcccgggccgccgacgccgagtgcgaggaacccttaattaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 53 - DNA, 2034 bp, synthetic construct
  source 1..2034: mol_type = other DNA
atggaggcccccgcccagctgctgttcctgctgctgctgtggctgcccgacaccaccggccaggtgcagctggtggagagcggcggcggcgtggtgcagcccggcagaagcctgagactgagctgcgccgccagcggcttcaccttcagcgactactacatgtactgggtgagacaggcccccggcaagggcctggagtgggtggccagcatcagcttcgacggcacctacacctactacaccgacagagtgaagggcagattcaccatcagcagagacaacagcaagaacaccctgtacctgcagatgaacagcctgagagccgaggacaccgccgtgtactactgcgccagagacagacccgcctggttcccctactggggccagggcaccctggtgaccgtgagcgccggcggcggcggcagcggcggcggcggcagcggcggcggcggcagcggcgacatcgtgatgacccagacccccctgagcctgagcgtgacccccggccagcccgccagcatcagctgcagaagcagccagagcatcgtgcacagcaacggcaacacctacctggagtggtacctgcagaagcccggccagagcccccagctgctgatctacaaggtgagcaacagattcagcggcgtgcccgacagattcagcggcagcggcagcggcaccgacttcaccctgaagatcagcagagtggaggccgaggacgtgggcgtgtactactgcttccagggcagccacgtgcccctgaccttcggcgccggcaccaagctggagctgaagggcgccccctcaccggtcaaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaagacaagcagaataccgtgtgtgaagagtgccccgacggcacctacagcgacgaggccaaccacgtggacccctgcctgccctgcactgtgtgcgaggacaccgagcggcagctgcgcgagtgcacaagatgggccgacgccgagtgcgaagagatccccggcagatggatcaccagaagcaccccccctgagggcagcgacagcaccgcccctagcacccaggaacctgaggcccctcccgagcaggacctgatcgcctctacagtggccggcgtggtgacaaccgtgatgggcagctctcagcccgtggtgacacggggcaccaccgacaatcccttaattaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 54 - DNA, 1413 bp, synthetic construct
  source 1..1413: mol_type = other DNA
atggaggcccccgcccagctgctgttcctgctgctgctgtggctgcccgacaccaccggccaggtgcagctggtggagagcggcggcggcgtggtgcagcccggcagaagcctgagactgagctgcgccgccagcggcttcaccttcagcgactactacatgtactgggtgagacaggcccccggcaagggcctggagtgggtggccagcatcagcttcgacggcacctacacctactacaccgacagagtgaagggcagattcaccatcagcagagacaacagcaagaacaccctgtacctgcagatgaacagcctgagagccgaggacaccgccgtgtactactgcgccagagacagacccgcctggttcccctactggggccagggcaccctggtgaccgtgagcgccggcggcggcggcagcggcggcggcggcagcggcggcggcggcagcggcgacatcgtgatgacccagacccccctgagcctgagcgtgacccccggccagcccgccagcatcagctgcagaagcagccagagcatcgtgcacagcaacggcaacacctacctggagtggtacctgcagaagcccggccagagcccccagctgctgatctacaaggtgagcaacagattcagcggcgtgcccgacagattcagcggcagcggcagcggcaccgacttcaccctgaagatcagcagagtggaggccgaggacgtgggcgtgtactactgcttccagggcagccacgtgcccctgaccttcggcgccggcaccaagctggagctgaagggcgccccctcaccggtcgagcccaagagctgcgacaagacccacacctgtcccccctgcccccccttaattaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaa

SEQ ID NO: 55 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 56 - DNA, 1005 bp, synthetic construct
  source 1..1005: mol_type = other DNA
agaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgtttatg

SEQ ID NO: 57 - protein, 17 aa, synthetic construct
ATSSGESKYGPPCPPCP

SEQ ID NO: 58 - protein, 117 aa, synthetic construct
QVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSA

SEQ ID NO: 59 - protein, 115 aa, synthetic construct
DIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKGAP

SEQ ID NO: 60 - protein, 41 aa, synthetic construct
RSKRSRLLHSDYMNMTPRRPGPTRKHYQPYAPPRDFAAYRS

SEQ ID NO: 61 - protein, 27 aa, synthetic construct
FWVLVVVGGVLACYSLLVTVAFIIFWV

SEQ ID NO: 62 - protein, 4 aa, synthetic construct
GGGS

SEQ ID NO: 63 - protein, 5 aa, synthetic construct
GGGGS

SEQ ID NO: 64 - protein, 16 aa, synthetic construct
GGGGSGGGGSGGGGSG

SEQ ID NO: 65 - protein, 15 aa, synthetic construct
GGGGSGGGGSGGGGS

SEQ ID NO: 66 - protein, 653 aa, Homo sapiens
AAAALGSSSGSASPAVAELCQNTPETFLEASKLLLTYADNILRNPNDEKYRSIRIGNTAFSTRLLPVRGAVECLFEMGFEEGETHLIFPKKASVEQLQKIRDLIAIERSSRLDGSNKSHKVKSSQQPAASTQLPTTPSSNPSGLNQHTRNRQGQSSDPPSASTVAADSAILEVLQSNIQHVLVYENPALQEKALACIPVQELKRKSQEKLSRARKLDKGINISDEDFLLLELLHWFKEEFFHWVNNVLCSKCGGQTRSRDRSLLPSDDELKWGAKEVEDHYCDACQFSNRFPRYNNPEKLLETRCGRCGEWANCFTLCCRAVGFEARYVWDYTDHVWTEVYSPSQQRWLHCDACEDVCDKPLLYEIGWGKKLSYVIAFSKDEVVDVTWRYSCKHEEVIARRTKVKEALLRDTINGLNKQRQLFLSENRRKELLQRIIVELVEFISPKTPKPGELGGRISGSVAWRVARGEMGLQRKETLFIPCENEKISKQLHLCYNIVKDRYVRVSNNNQTISGWENGVWKMESIFRKVETDWHMVYLARKEGSSFAYISWKFECGSVGLKVDSISIRTSSQTFQTGTVEWKLRSDTAQVELTGDNSLHSYADFSGATEVILEAELSRGDGDVAWQHTQLFRQSLNDHEENCLEIIIKFSDL

SEQ ID NO: 67 - protein, 563 aa, synthetic construct
KASVEQLQKIRDLIAIERSSRLDGSNKSHKVKSSQQPAASTQLPTTPSSNPSGLNQHTRNRQGQSSDPPSASTVAADSAILEVLQSNIQHVLVYENPALQEKALACIPVQELKRKSQEKLSRARKLDKGINISDEDFLLLELLHWFKEEFFHWVNNVLCSKCGGQTRSRDRSLLPSDDELKWGAKEVEDHYCDACQFSNRFPRYNNPEKLLETRCGRCGEWANCFTLCCRAVGFEARYVWDYTDHVWTEVYSPSQQRWLHCDACEDVCDKPLLYEIGWGKKLSYVIAFSKDEVVDVTWRYSCKHEEVIARRTKVKEALLRDTINGLNKQRQLFLSENRRKELLQRIIVELVEFISPKTPKPGELGGRISGSVAWRVARGEMGLQRKETLFIPCENEKISKQLHLCYNIVKDRYVRVSNNNQTISGWENGVWKMESIFRKVETDWHMVYLARKEGSSFAYISWKFECGSVGLKVDSISIRTSSQTFQTGTVEWKLRSDTAQVELTGDNSLHSYADFSGATEVILEAELSRGDGDVAWQHTQLFRQSLNDHEENCLEIIIKFSDL

SEQ ID NO: 68 - protein, 362 aa, synthetic construct
KASVEQLQKIRDLIAIERSSRLDGSNKSHKVKSSQQPAASTQLPTTPSSNPSGLNQHTRNRQGQSSDPPSASTVAADSAILEVLQSNIQHVLVYENPALQEKALACIPVQELKRKSQEKLSRARKLDKGINISDEDFLLLELLHWFKEEFFHWVNNVLCSKCGGQTRSRDRSLLPSDDELKWGAKEVEDHYCDACQFSNRFPRYNNPEKLLETRCGRCGEWANCFTLCCRAVGFEARYVWDYTDHVWTEVYSPSQQRWLHCDACEDVCDKPLLYEIGWGKKLSYVIAFSKDEVVDVTWRYSCKHEEVIARRTKVKEALLRDTINGLNKQRQLFLSENRRKELLQRIIVELVEFISPKTPKPG

SEQ ID NO: 69 - protein, 452 aa, synthetic construct
AAAALGSSSGSASPAVAELCQNTPETFLEASKLLLTYADNILRNPNDEKYRSIRIGNTAFSTRLLPVRGAVECLFEMGFEEGETHLIFPKKASVEQLQKIRDLIAIERSSRLDGSNKSHKVKSSQQPAASTQLPTTPSSNPSGLNQHTRNRQGQSSDPPSASTVAADSAILEVLQSNIQHVLVYENPALQEKALACIPVQELKRKSQEKLSRARKLDKGINISDEDFLLLELLHWFKEEFFHWVNNVLCSKCGGQTRSRDRSLLPSDDELKWGAKEVEDHYCDACQFSNRFPRYNNPEKLLETRCGRCGEWANCFTLCCRAVGFEARYVWDYTDHVWTEVYSPSQQRWLHCDACEDVCDKPLLYEIGWGKKLSYVIAFSKDEVVDVTWRYSCKHEEVIARRTKVKEALLRDTINGLNKQRQLFLSENRRKELLQRIIVELVEFISPKTPKPG

SEQ ID NO: 70 - protein, 297 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 71 - protein, 21 aa, Homo sapiens
MALPVTALLLPLALLLHAARP

SEQ ID NO: 72 - protein, 19 aa, Homo sapiens
MEFGLSWVFLVALLRGVQC

SEQ ID NO: 73 - protein, 17 aa, Homo sapiens
MWLQSLLLLGTVACSIS

SEQ ID NO: 74 - protein, 20 aa, Homo sapiens
MEAPAQLLFLLLLWLPDTTG

SEQ ID NO: 75 - protein, 22 aa, Homo sapiens
MLLLVTSLLLCELPHPAFLLIP

SEQ ID NO: 76 - protein, 674 aa, synthetic construct
MALPVTALLLPLALLLHAARPAAAALGSSSGSASPAVAELCQNTPETFLEASKLLLTYADNILRNPNDEKYRSIRIGNTAFSTRLLPVRGAVECLFEMGFEEGETHLIFPKKASVEQLQKIRDLIAIERSSRLDGSNKSHKVKSSQQPAASTQLPTTPSSNPSGLNQHTRNRQGQSSDPPSASTVAADSAILEVLQSNIQHVLVYENPALQEKALACIPVQELKRKSQEKLSRARKLDKGINISDEDFLLLELLHWFKEEFFHWVNNVLCSKCGGQTRSRDRSLLPSDDELKWGAKEVEDHYCDACQFSNRFPRYNNPEKLLETRCGRCGEWANCFTLCCRAVGFEARYVWDYTDHVWTEVYSPSQQRWLHCDACEDVCDKPLLYEIGWGKKLSYVIAFSKDEVVDVTWRYSCKHEEVIARRTKVKEALLRDTINGLNKQRQLFLSENRRKELLQRIIVELVEFISPKTPKPGELGGRISGSVAWRVARGEMGLQRKETLFIPCENEKISKQLHLCYNIVKDRYVRVSNNNQTISGWENGVWKMESIFRKVETDWHMVYLARKEGSSFAYISWKFECGSVGLKVDSISIRTSSQTFQTGTVEWKLRSDTAQVELTGDNSLHSYADFSGATEVILEAELSRGDGDVAWQHTQLFRQSLNDHEENCLEIIIKFSDL

SEQ ID NO: 77 - protein, 672 aa, synthetic construct
MEFGLSWVFLVALLRGVQCAAAALGSSSGSASPAVAELCQNTPETFLEASKLLLTYADNILRNPNDEKYRSIRIGNTAFSTRLLPVRGAVECLFEMGFEEGETHLIFPKKASVEQLQKIRDLIAIERSSRLDGSNKSHKVKSSQQPAASTQLPTTPSSNPSGLNQHTRNRQGQSSDPPSASTVAADSAILEVLQSNIQHVLVYENPALQEKALACIPVQELKRKSQEKLSRARKLDKGINISDEDFLLLELLHWFKEEFFHWVNNVLCSKCGGQTRSRDRSLLPSDDELKWGAKEVEDHYCDACQFSNRFPRYNNPEKLLETRCGRCGEWANCFTLCCRAVGFEARYVWDYTDHVWTEVYSPSQQRWLHCDACEDVCDKPLLYEIGWGKKLSYVIAFSKDEVVDVTWRYSCKHEEVIARRTKVKEALLRDTINGLNKQRQLFLSENRRKELLQRIIVELVEFISPKTPKPGELGGRISGSVAWRVARGEMGLQRKETLFIPCENEKISKQLHLCYNIVKDRYVRVSNNNQTISGWENGVWKMESIFRKVETDWHMVYLARKEGSSFAYISWKFECGSVGLKVDSISIRTSSQTFQTGTVEWKLRSDTAQVELTGDNSLHSYADFSGATEVILEAELSRGDGDVAWQHTQLFRQSLNDHEENCLEIIIKFSDL

SEQ ID NO: 78 - protein, 582 aa, synthetic construct
MEFGLSWVFLVALLRGVQCKASVEQLQKIRDLIAIERSSRLDGSNKSHKVKSSQQPAASTQLPTTPSSNPSGLNQHTRNRQGQSSDPPSASTVAADSAILEVLQSNIQHVLVYENPALQEKALACIPVQELKRKSQEKLSRARKLDKGINISDEDFLLLELLHWFKEEFFHWVNNVLCSKCGGQTRSRDRSLLPSDDELKWGAKEVEDHYCDACQFSNRFPRYNNPEKLLETRCGRCGEWANCFTLCCRAVGFEARYVWDYTDHVWTEVYSPSQQRWLHCDACEDVCDKPLLYEIGWGKKLSYVIAFSKDEVVDVTWRYSCKHEEVIARRTKVKEALLRDTINGLNKQRQLFLSENRRKELLQRIIVELVEFISPKTPKPGELGGRISGSVAWRVARGEMGLQRKETLFIPCENEKISKQLHLCYNIVKDRYVRVSNNNQTISGWENGVWKMESIFRKVETDWHMVYLARKEGSSFAYISWKFECGSVGLKVDSISIRTSSQTFQTGTVEWKLRSDTAQVELTGDNSLHSYADFSGATEVILEAELSRGDGDVAWQHTQLFRQSLNDHEENCLEIIIKFSDL

SEQ ID NO: 79 - protein, 381 aa, synthetic construct
MEFGLSWVFLVALLRGVQCKASVEQLQKIRDLIAIERSSRLDGSNKSHKVKSSQQPAASTQLPTTPSSNPSGLNQHTRNRQGQSSDPPSASTVAADSAILEVLQSNIQHVLVYENPALQEKALACIPVQELKRKSQEKLSRARKLDKGINISDEDFLLLELLHWFKEEFFHWVNNVLCSKCGGQTRSRDRSLLPSDDELKWGAKEVEDHYCDACQFSNRFPRYNNPEKLLETRCGRCGEWANCFTLCCRAVGFEARYVWDYTDHVWTEVYSPSQQRWLHCDACEDVCDKPLLYEIGWGKKLSYVIAFSKDEVVDVTWRYSCKHEEVIARRTKVKEALLRDTINGLNKQRQLFLSENRRKELLQRIIVELVEFISPKTPKPG

SEQ ID NO: 80 - protein, 376 aa, synthetic construct
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 81 - DNA, 1131 bp, synthetic construct
  source 1..1131: mol_type = other DNA
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggcgagatatcggccggggacgcggcggtggtaatgacaagcgcccagataacaatgggcatgccttatgccgtgaccgacgccgttctggctcctcatgtcgggggggaggctgggagttcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggtgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactga

SEQ ID NO: 82 - DNA, 1131 bp, synthetic construct
  source 1..1131: mol_type = other DNA
atggccagctatccttgtcaccagcacgccagcgcctttgatcaggccgcaagatctagaggccacagcaacagaagaacagccctgcggcctcggagacagcaagaggctacagaagttcggctggaacagaagatgcccacactgctgcgggtgtacatcgatggccctcacggcatgggcaagaccaccacaacacagctgctggtggccctgggcagcagagatgacatcgtgtatgtgcccgagcctatgacctactggcaggttctgggagccagcgagacaatcgccaacatctacaccacacagcaccggctggatcagggcgaaatttctgctggcgacgccgccgtggttatgacatctgcccagatcaccatgggcatgccttacgccgtgacagatgctgtgctggcccctcatgttggcggagaagccggatcttctcatgcccctccaccagctctgaccctgatcttcgacagacaccctatcgccgctctgctgtgttatcctgccgccagatacctgatgggcagcatgacacctcaggccgtgctggctttcgtggccctgattcctcctacactgcccggcaccaatatcgtgctgggagccctgcctgaggaccggcacattgatagactggccaagagacagcggcctggcgagagactggatctggctatgctggccgccatcagaagagtgtacggcctgctggccaacaccgtgcggtatcttcaaggcggaggatcttggtgggaagattggggccagctgtctggcacagcagttcctccacaaggcgccgagcctcagtctaatgctggacccagacctcacatcggcgacaccctgtttaccctgttcagagcccctgagctgctggctcctaacggcgacctgtacaacgtgttcgcctgggctcttgacgtgctggcaaaaagactgcggcccatgcacgtgttcatcctggactacgatcagtcccctgccggctgtagagatgctctgctgcagctgacaagcggcatggtgcagacccacgttacaacccctggcagcatccccaccatctgtgacctggccagaaccttcgccagagagatgggcgaagccaactga

SEQ ID NO: 83 - protein, 404 aa, synthetic construct
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 84 - DNA, 1215 bp, synthetic construct
  source 1..1215: mol_type = other DNA
atgctggaaggcgtgcaggtcgagacaatctctcctggcgacggcagaacattccccaagaggggccagacatgcgtggtgcactataccggcatgctcgaggacggcaagaaggtggacagcagccgggacagaaacaagcccttcaagttcatgctgggcaagcaagaagtgatcagaggctgggaagagggcgtcgcccagatgtctgttggacagagagccaagctgacaatcagccccgattacgcctatggcgccacaggacaccctggcatcattcctccacatgccacactggtgttcgacgtggaactgctgaagctggaaagcggcggaggatctggcgtcgacggatttggagatgtgggcgccctggaaagcctgagaggaaatgccgatctggcctacatcctgagcatggaaccttgcggccactgcctgattatcaacaacgtgaacttctgcagagagagcggcctgagaaccagaaccggcagcaacatcgactgcgagaagctgcggagaagattcagcagcctgcacttcatggtggaagtgaagggcgacctgaccgccaagaaaatggtgctggctctgctggaactggcccagcaagatcatggcgctctggattgctgcgtggtcgtgatcctgtctcatggctgtcaggccagccatctgcaattccctggcgccgtgtatggcaccgatggctgtcctgtgtccgtggaaaagatcgtgaacatcttcaacggcaccagctgtcctagcctcggcggaaagcccaagctgttcttcatccaagcctgtggcggcgagcagaaggatcacggatttgaggtggccagcacaagccccgaggatgagtctcctggaagcaaccctgagcctgacgccacacctttccaagagggactgagaaccttcgaccagctggacgctatcagctccctgcctacacctagcgacatcttcgtgtcctacagcacattccccggctttgtgtcttggcgggatcccaagtctggctcttggtacgtggaaaccctggacgacatctttgagcagtgggcccatagcgaggacctgcagtctctgctgctgagagtggccaatgccgtgtccgtgaagggcatctacaagcagatgcctggctgcttcaacttcctgcggaagaagctgtttttcaagaccagcgctagccgggcctga

SEQ ID NO: 85 - DNA, 1551 bp, synthetic construct
  source 1..1551: mol_type = other DNA
atggaagctcctgctcagctgctgtttctgctgctgctgtggctgcctgataccaccggcgacatcgtgatgacacagagccctgatagcctggccgtgtctctgggagagagagccaccatcaactgcagaagcagccagagcctgctgcacagctccaaccagagaaactacctggcctggtatcagcagaagcccggccagcctcctaaggtgctgatctactgggccagcaccagagaatccggcgtgcccgatagattttctggctctggcagcggcaccgacttcaccctgacaatttctagcctgcaagccgaggacgtggccgtgtactactgccagcagtactacagctacccctggacctttggccagggcaccaaggtggaaatcaaaggcggcggaggatctggcggaggtggaagtggcggaggcggatctcaagttcagctggttcagtctggcgccgaagtgaagaaacctggcgcctctgtgaaggtgtcctgcaaggcctctggctacaccctgaccgatcacaccatccactggatgagacaggcccctggccagagactggaatggatcggctacatctaccccagagatggcatcaccggctacaacgagcggttcagaggaaaggccacactgaccgccgatacaagcgccagcacagcctacatggaactgagcagcctgagaagcgaggacaccgccgtgtattattgcgccagatggggctactcctaccggaactacgcctactactacgactactggggccagggaaccctggtcacagtttctagcgccgcagccacaagctccggcgagtctaaatacggccctccttgtccgccttgtcctgacatcttttgggtgctcgtggtcgttggcggagtgctggcctgttatagcctgctggttaccgtggcctttatcatcttctgggtccgaagcaagcggagcagactgctgcactccgactacatgaacatgacccctagacggcccggacctaccagaaagcactaccagccttacgctcctcctagagacttcgccgcctacagaagcgcctccaagagaggcagaaagaagctgctgtacatcttcaagcagcccttcatgcggcccgtgcagaccacacaagaggaagatggctgcagctgtcggttccccgaggaagaagaaggcggctgcgagctgagagtgaagttcagcagatccgccgacgctcccgcctataagcagggacagaatcagctgtacaatgagctgaacctggggcgcagagaagagtacgacgtgctggacaagagaagaggcagggaccctgagatgggcggcaagcccagaagaaagaaccctcaagagggcctgtataacgagctgcagaaagacaagatggccgaggcctacagcgagatcggaatgaagggcgaacgccgcagaggcaagggacacgatggactgtatcagggcctgagcaccgccaccaaggatacctatgatgccctgcacatgcaggccctgccacctagataa

(19) United States
(12) Patent Application Publication
(10) Pub. No.: US 2026/0193371 A1
(43) Pub. Date: Jul. 9, 2026

(54) CDH17 CAR

(71) Applicants: Ospedale San Raffaele S.r.l., Milan (IT); Università Vita-Salute San Raffaele, Milan (IT)

(72) Inventors: Monica Casucci, Milan (IT); Maria Chiara Bonini, Milan (IT); Beatrice Greco, Milan (IT); Fedeica De Girardi, Milan (IT); Rita El Khoury, Milan (IT)

(21) Appl. No.: 19/129,003

(22) PCT Filed: Nov. 10, 2023
(86) PCT No.: PCT/EP2023/081532

§ 371 Date: May 10, 2025

(30) Foreign Application Priority Data
Nov. 11, 2022 (EP) 22207030.2

(51) Int. Cl. C07K 16/28; A61K 9/00; A61K 31/7088; A61K 35/17; A61K 38/00; A61K 40/11; A61K 40/31; A61K 40/42; A61P 35/04; C07K 14/705; C07K 14/725; C07K 14/73; C12N 15/62; C12N 15/63; G01N 33/575

(52) U.S. Cl. CPC C07K 16/2896; A61K 9/0019; A61K 31/7088; A61K 35/17; A61K 38/00; A61K 40/11; A61K 40/31; A61K 40/42; A61P 35/04; C07K 14/7051; C07K 14/70514; C07K 14/70517; C07K 14/70521; C12N 15/625; C12N 15/63; G01N 33/575; C07K 2317/53; C07K 2317/565; C07K 2317/622; C07K 2319/02; C07K 2319/03

(57) ABSTRACT

The present invention relates to chimeric antigen receptors (CARs) that target Cadherin-17 (CDH17) and cells comprising said CARs. Methods and uses involving the CARs of the invention are also provided.

### FIELD OF THE INVENTION

The present invention relates to chimeric antigen receptors (CARs) and cells comprising said CARs. Methods and uses involving the CARs of the invention are also provided.

### BACKGROUND TO THE INVENTION

Chimeric antigen receptors (CARs) are synthetic biology molecules commonly constructed by fusing an antigen-binding moiety, often derived from a tumor-reactive monoclonal antibody, with intracellular signaling domains derived from T lymphocytes. Clinical testing of CAR-T cell therapies has increased rapidly in recent years, leading to marketing authorizations for different CAR-T cell products targeting either CD19 or BCMA in relapsed/refractory B-cell lymphomas, B-cell acute lymphoblastic leukemia of children and young adults and multiple myeloma. Despite the successes of CAR-T therapies, objective response rates in patients with solid tumors are far less frequent and improving therapeutic efficacy against these malignancies represents one of the biggest challenges in the field.

Effective targeting of solid tumors requires overcoming a number of barriers, such as poor trafficking to the tumor site and suboptimal infiltration of tumor masses. Further difficulties arise due to the highly immunosuppressive tumor microenvironment, which leads to T-cell anergy, exhaustion, and senescence.

In addition to the barriers associated with targeting solid tumors, the choice of antigen is an important and challenging consideration. Most antigens that are expressed by solid malignancies are shared with healthy tissues, which increases the risk of detrimental on-target off-tumor toxicities. Ideal CAR targets are surface molecules that are highly and homogeneously expressed in tumor cells, but not in heathy tissues. In addition, and advantageously, a preferred antigen would be expressed by multiple tumor types and be involved in cancer cell survival to reduce antigen loss.

Thus, there remains an unmet need to identify new antigens for CAR cell therapy. Furthermore, there remains a need for CARs that effectively target relevant antigens and which may be used in therapy.

### SUMMARY OF THE INVENTION

The present inventors have identified an antigen, Cadherin-17 (CDH-17), and developed CARs that target CDH-17. The inventors have demonstrated that the CAR of the invention is able to redirect the specificity and functional activity of human T cells towards tumor cells expressing the CDH-17 target antigen.

Using a rational process, which integrated data from multiple sources, including the analysis of liver metastases from patients with colorectal cancer (CRC), the inventors identified new antigens suitable for CAR T-cell therapy, of which Cadherin-17 (CDH-17) or Li-cadherin, was the most promising candidate. The inventors subsequently devised and generated CAR constructs targeting CDH-17, which exhibit high reactivity against CDH17+ tumors.

The CARs of the present invention may be used to generate CAR-containing cells, such as CAR T-cells specific for the CDH-17 antigen, which can be applied to the treatment of different gastrointestinal tumors, including colorectal, pancreatic and stomach cancers. Importantly, the CAR cells of the invention may target liver metastases (LV MTS) of colorectal cancer and pancreatic ductal adenocarcinoma (PDAC), which represent a major cause of death and an important but unmet clinical need.

In one aspect, there is provided a chimeric antigen receptor (CAR) comprising an antigen-binding domain, wherein the antigen-binding domain comprises:
a) heavy chain variable region (VH) complementarity determining regions (CDRs) with the sequences: CDR1—DHTIHWMR (SEQ ID NO: 1); CDR2—YIYPRDGITGYNERFRGK (SEQ ID NO: 2); and CDR3—WGYSYRNYAYYYDYWGQGTL (SEQ ID NO: 3); or variants thereof each having up to three amino acid substitutions, additions or deletions; and
b) light chain variable region (VL) CDRs with the sequences: CDR1—INCRSSQSLLHSSNQR (SEQ ID NO: 4); CDR2—PPKVLIYWASTRES (SEQ ID NO: 5); and CDR3—QQYYSYPWTFGQ (SEQ ID NO: 6); or variants thereof each having up to three amino acid substitutions, additions or deletions.

Preferably, the antigen-binding domain binds CDH-17.

In one embodiment, the antigen-binding domain comprises:
a) a VH domain comprising the sequence of SEQ ID NO: 7; and
b) a VL domain comprising the sequence of SEQ ID NO: 8.

In one embodiment, the antigen-binding domain comprises:
a) a VH domain comprising the sequence of SEQ ID NO: 7; and
b) a VL domain comprising the sequence of SEQ ID NO: 8;
   or variants thereof, each having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the antigen-binding domain comprises:
a) a VH domain comprising the sequence of SEQ ID NO: 7; and
b) a VL domain comprising the sequence of SEQ ID NO: 8;
   or variants thereof, each having at least 90% sequence identity thereto.

In one embodiment, the antigen-binding domain comprises a single-chain variable fragment (scFv).

In one embodiment, the antigen-binding domain consists of a single-chain variable fragment (scFv).

In one embodiment, the antigen-binding domain comprises the sequence of SEQ ID NO: 9.

In one embodiment, the antigen-binding domain consists of the sequence of SEQ ID NO: 9.

In one embodiment, the antigen-binding domain comprises a sequence having at least 90% sequence identity to the sequence of SEQ ID NO: 9.

In one embodiment, the antigen-binding domain comprises or consists of a sequence having at least 75% sequence identity to the sequence of SEQ ID NO: 9, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 9.

In another aspect, there is provided a chimeric antigen receptor (CAR) comprising an antigen-binding domain, wherein the antigen-binding domain comprises:
a) heavy chain variable region (VH) complementarity determining regions (CDRs) with the sequences: CDR1—DYYMY (SEQ ID NO: 42); CDR2—SISFDGTYTYYTDRVKG (SEQ ID NO: 43); and CDR3—DRPAWFPY (SEQ ID NO: 44); or variants thereof each having up to three amino acid substitutions, additions or deletions; and
b) light chain variable region (VL) CDRs with the sequences: CDR1—RSSQSIVHSNGNTYLE (SEQ ID NO: 39); CDR2—KVSNRFS (SEQ ID NO: 40); and CDR3—FQGSHVPLT (SEQ ID NO: 41); or variants thereof each having up to three amino acid substitutions, additions or deletions.

In one embodiment, the antigen-binding domain comprises:
a) a VH domain comprising the sequence of SEQ ID NO: 58; and
b) a VL domain comprising the sequence of SEQ ID NO: 59.

In one embodiment, the antigen-binding domain comprises:
a) a VH domain comprising the sequence of SEQ ID NO: 58; and
b) a VL domain comprising the sequence of SEQ ID NO: 59;
   or variants thereof, each having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the antigen-binding domain comprises:
a) a VH domain comprising the sequence of SEQ ID NO: 58; and
b) a VL domain comprising the sequence of SEQ ID NO: 59;
   or variants thereof, each having at least 90% sequence identity thereto.

In one embodiment, the antigen-binding domain comprises a single-chain variable fragment (scFv).

In one embodiment, the antigen-binding domain consists of a single-chain variable fragment (scFv).

In one embodiment, the antigen-binding domain comprises the sequence of SEQ ID NO: 45.

In one embodiment, the antigen-binding domain consists of the sequence of SEQ ID NO: 45.

In one embodiment, the antigen-binding domain comprises a sequence having at least 90% sequence identity to the sequence of SEQ ID NO: 45.

In one embodiment, the antigen-binding domain comprises or consists of a sequence having at least 75% sequence identity to the sequence of SEQ ID NO: 45, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 45.

In one embodiment, the CAR comprises a CD28, CD8, and/or CD4 transmembrane domain.

In one embodiment, the CAR comprises a CD28 transmembrane domain.

In one embodiment, the CD28 transmembrane domain comprises the sequence of SEQ ID NO: 61.

In one embodiment, the CD28 transmembrane domain consists of the sequence of SEQ ID NO: 61.

In one embodiment, the CD28 transmembrane domain comprises a sequence having at least 90% sequence identity to the sequence of SEQ ID NO: 61.

In one embodiment, the CD28 transmembrane domain comprises or consists of a sequence having at least 75% sequence identity to the sequence of SEQ ID NO: 61, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 61.

In one embodiment, the CAR comprises CD28 transmembrane and co-stimulatory domain.

In one embodiment, the CD28 transmembrane domain and co-stimulatory domain are not separated by another sequence.

In one embodiment, the CD28 transmembrane domain and co-stimulatory domain comprise the sequence of SEQ ID NO: 12.

In one embodiment, the CD28 transmembrane domain and co-stimulatory domain consist of the sequence of SEQ ID NO: 12.

In one embodiment, the CD28 transmembrane domain and co-stimulatory domain comprise a sequence having at least 90% sequence identity to the sequence of SEQ ID NO: 12.

In one embodiment, the CD28 transmembrane domain and co-stimulatory domain comprise a sequence having at least 75% sequence identity to the sequence of SEQ ID NO: 12, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 12.

In one embodiment, the CAR comprises a CD8 transmembrane domain.

In one embodiment, the CAR comprises a CD4 transmembrane domain.

In one embodiment, the CAR comprises an IgG1 hinge, a LNGFR spacer, or a mCH2CH3 spacer.

In one embodiment, the CAR comprises an IgG1 hinge.

In one embodiment, the IgG1 hinge comprises the sequence of SEQ ID NO: 10.

In one embodiment, the IgG1 hinge consists of the sequence of SEQ ID NO: 10.

In one embodiment, the IgG1 hinge comprises a sequence having at least 90% sequence identity to the sequence of SEQ ID NO: 10.

In one embodiment, the IgG1 hinge comprises or consists of a sequence having at least 75% sequence identity to the sequence of SEQ ID NO: 10, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 10.

In one embodiment, the CAR comprises a LNGFR spacer.

In one embodiment, the LNGFR spacer is a LNGFR mutated short (NMS) spacer. In another embodiment, the LNGFR spacer is a LNGFR wild type long spacer (NWL).

In one embodiment, the LNGFR spacer comprises the sequence of SEQ ID NO: 11 or 27.

In one embodiment, the LNGFR spacer consists of the sequence of SEQ ID NO: 11 or 27.

In one embodiment, the LNGFR spacer comprises a sequence having at least 90% sequence identity to the sequence of SEQ ID NO: 11 or 27.

In one embodiment, the LNGFR spacer comprises a sequence having at least 90% sequence identity to the sequence of any one of SEQ ID NO: 11, 27, 32, or 33.

In one embodiment, the LNGFR spacer comprises or consists of a sequence having at least 75% sequence identity to the sequence of SEQ ID NO: 11, 27, 32, or 33, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 11, 27, 32, or 33.

In one embodiment, the CAR comprises a mCH2CH3 spacer.

In one embodiment, the mCH2CH3 spacer comprises the sequence of SEQ ID NO: 28.

In one embodiment, the mCH2CH3 spacer consists of the sequence of SEQ ID NO: 28.

In one embodiment, the mCH2CH3 spacer comprises a sequence having at least 90% sequence identity to the sequence of SEQ ID NO: 28.

In one embodiment, the mCH2CH3 spacer comprises or consists of a sequence having at least 75% sequence identity to the sequence of SEQ ID NO: 28, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 28.

In one embodiment, the CAR comprises a CD28 and/or a 4-1BB co-stimulatory domain.

In one embodiment, the CAR comprises a CD28 co-stimulatory domain.

In one embodiment, the co-stimulatory domain is a CD28 co-stimulatory domain.

In one embodiment, the CD28 co-stimulatory domain comprises the sequence of SEQ ID NO: 60.

In one embodiment, the CD28 co-stimulatory domain consist of the sequence of SEQ ID NO: 60.

In one embodiment, the CD28 co-stimulatory domain comprises a sequence having at least 90% sequence identity to the sequence of SEQ ID NO: 60.

In one embodiment, the CD28 co-stimulatory domain comprises or consists of a sequence having at least 75% sequence identity to the sequence of SEQ ID NO: 60, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 60.

In one embodiment, the CAR comprises a 4-1BB co-stimulatory domain.

In one embodiment, the CAR comprises a CD3-zeta signalling domain.

In one embodiment, the CD3-zeta signalling domain comprises the sequence of SEQ ID NO: 13.

In one embodiment, the CD3-zeta signalling domain consists of the sequence of SEQ ID NO: 13.

In one embodiment, the CD3-zeta signalling domain comprises a sequence having at least 90% sequence identity to the sequence of SEQ ID NO: 13.

In one embodiment, the CD3-zeta signalling domain comprises or consists of a sequence having at least 75% sequence identity to the sequence of SEQ ID NO: 13, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 13.

In one embodiment, the CAR comprises:
a) a CD28, a CD8, and/or a CD4 transmembrane domain;
b) an IgG1 hinge, LNGFR spacer or mCH2CH3 spacer;
c) a CD28 and/or a 4-1BB co-stimulatory domain; and/or
d) a CD3-zeta signalling domain.

In one embodiment, the CAR comprises:
a) a transmembrane domain derived from a CD28, a CD8, and/or a CD4 transmembrane domain;
b) a spacer domain comprising an IgG1 hinge, LNGFR spacer or mCH2CH3 spacer;
c) one or more co-stimulatory domain selected from the group consisting of: a CD28 and/or a 4-1BB co-stimulatory domain; and/or
d) a CD3-zeta signalling domain.

In one embodiment, the CAR comprises or consists of a sequence having at least 75% sequence identity to the sequence of any one of SEQ ID NOs: 9, 14, 15, 23, 24, or 47, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of any one of SEQ ID NOs: 9, 14, 15, 23, 24, or 47.

In one embodiment, the CAR comprises a sequence having at least 90% sequence identity to the sequence of any one of SEQ ID NOs: 9, 14, 15, 23, 24, or 47.

In one embodiment, the CAR consists of a sequence having at least 90% sequence identity to the sequence of any one of SEQ ID NOs: 14, 15, 23, 24, or 47.

In one embodiment, the CAR comprises the sequence of any one of SEQ ID NOs: 9, 14, 15, 23, 24, or 47.

In one embodiment, the CAR comprises a sequence having at least 90% sequence identity to the sequence of any one of SEQ ID NOs: 9, 14, 15, 23, 24, or 47, wherein said sequence comprises any one or more of the sequences according to SEQ ID NOs: 1-6.

In one embodiment, the CAR comprises a sequence having at least 90% sequence identity to the sequence of any one of SEQ ID NOs: 9, 14, 15, 23, 24, or 47, wherein said sequence comprises the sequences according to SEQ ID NOs: 1-6.

In one embodiment, the CAR comprises a sequence having at least 90% sequence identity to the sequence of any one of SEQ ID NOs: 45, or 48-50.

In one embodiment, the CAR consists of a sequence having at least 90% sequence identity to the sequence of any one of SEQ ID NOs: 48-50.

In one embodiment, the CAR comprises the sequence of any one of SEQ ID NOs: 45, or 48-50.

In one embodiment, the CAR comprises a sequence having at least 90% sequence identity to the sequence of any one of SEQ ID NOs: 45, or 48-50, wherein said sequence comprises any one or more of the sequences according to SEQ ID NOs: 39-44.

In one embodiment, the CAR comprises a sequence having at least 90% sequence identity to the sequence of any one of SEQ ID NOs: 45, or 48-50, wherein said sequence comprises the sequences according to SEQ ID NOs: 39-44.

In one aspect, there is provided a single-chain variable fragment (scFv) comprising:
a) heavy chain variable region (VH) complementarity determining regions (CDRs) with the sequences: CDR1—DHTIHWMR (SEQ ID NO: 1); CDR2—YIYPRDGITGYNERFRGK (SEQ ID NO: 2); and CDR3—WGYSYRNYAYYYDYWGQGTL (SEQ ID NO: 3); or variants thereof each having up to three amino acid substitutions, additions or deletions; and
b) light chain variable region (VL) CDRs with the sequences: CDR1—INCRSSQSLLHSSNQR (SEQ ID NO: 4); CDR2—PPKVLIYWASTRES (SEQ ID NO: 5); and CDR3—QQYYSYPWTFGQ (SEQ ID NO: 6); or variants thereof each having up to three amino acid substitutions, additions or deletions.

In one embodiment, the scFv comprises the sequence of SEQ ID NO: 9.

In one embodiment, the scFv consists of the sequence of SEQ ID NO: 9.

In one embodiment, the scFv comprises a sequence having at least 90% sequence identity to the sequence of SEQ ID NO: 9.

In one embodiment, the scFv comprises or consists of a sequence having at least 75% sequence identity to the sequence of SEQ ID NO: 9, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 9.

In one aspect, there is provided a single-chain variable fragment (scFv) comprising:
a) heavy chain variable region (VH) complementarity determining regions (CDRs) with the sequences: CDR1—DYYMY (SEQ ID NO: 42); CDR2—SISFDGTYTYYTDRVKG (SEQ ID NO: 43); and CDR3—DRPAWFPY (SEQ ID NO: 44); or variants thereof each having up to three amino acid substitutions, additions or deletions; and
b) light chain variable region (VL) CDRs with the sequences: CDR1—RSSQSIVHSNGNTYLE (SEQ ID NO: 39); CDR2—KVSNRFS (SEQ ID NO: 40); and CDR3—FQGSHVPLT (SEQ ID NO: 41); or variants thereof each having up to three amino acid substitutions, additions or deletions.

In one embodiment, the scFv comprises the sequence of SEQ ID NO: 45.

In one embodiment, the scFv consists of the sequence of SEQ ID NO: 45.

In one embodiment, the scFv comprises a sequence having at least 90% sequence identity to the sequence of SEQ ID NO: 45.

In one embodiment, the scFv comprises or consists of a sequence having at least 75% sequence identity to the sequence of SEQ ID NO: 45, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 45.

In one aspect, there is provided a polynucleotide comprising one or more nucleotide sequence encoding the CAR, or the scFv of the invention.

In one embodiment, the polynucleotide comprises a sequence encoding the scFv that comprises the sequence of SEQ ID NO: 16, or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the scFv that comprises the sequence of SEQ ID NO: 16.

In one embodiment, the polynucleotide comprises a sequence encoding the CAR that comprises the sequence of any one of SEQ ID NOs: 16-18, 25-26, or 51 or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the CAR that comprises the sequence of any one of SEQ ID NOs: 16-18, 25-26, or 51.

In one embodiment, the polynucleotide comprises:
a) a sequence encoding the scFv that comprises the sequence of SEQ ID NO: 16; and/or
b) a sequence encoding the CAR that comprises the sequence of any one of SEQ ID NOs: 16-18, 25-26, or 51;
   or variants thereof, each having at least 75% sequence identity thereto.

In one embodiment, the polynucleotide comprises a sequence encoding the CAR that comprises the sequence of any one of SEQ ID NOs: 16-18, 25-26, 51 or 85 or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the CAR that comprises the sequence of any one of SEQ ID NOs: 16-18, 25-26, 51 or 85.

In one embodiment, the polynucleotide comprises:
a) a sequence encoding the scFv that comprises the sequence of SEQ ID NO: 16; and/or
b) a sequence encoding the CAR that comprises the sequence of any one of SEQ ID NOs: 16-18, 25-26, 51 or 85;
   or variants thereof, each having at least 75% sequence identity thereto.

In one embodiment, the polynucleotide comprises a sequence encoding the scFv that comprises the sequence of SEQ ID NO: 46, or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the scFv that comprises the sequence of SEQ ID NO: 46.

In one embodiment, the polynucleotide comprises a sequence encoding the CAR that comprises the sequence of any one of SEQ ID NOs: 46, or 52-54, or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the CAR that comprises the sequence of any one of SEQ ID NOs: 46, or 52-54.

In one embodiment, the polynucleotide comprises:
a) a sequence encoding the scFv that comprises the sequence of SEQ ID NO: 46; and/or
b) a sequence encoding the CAR that comprises the sequence of any one of SEQ ID NOs: 46, or 52-54;
   or variants thereof, each having at least 75% sequence identity thereto.

In one embodiment, the polynucleotide comprises a sequence encoding the IgG1 hinge that comprises or consists of the sequence of SEQ ID NO: 19 or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the LNGFR spacer that comprises or consists of the sequence of SEQ ID NO: 20 or 29, or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the mCH2CH3 spacer that comprises or consists of the sequence of SEQ ID NO: 30, or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the CD28 co-stimulatory domain and transmembrane domain that comprises or consists of the sequence of SEQ ID NO: 21 or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the CD3-zeta signalling domain that comprises or consists of the sequence of SEQ ID NO: 22 or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide further encodes one or more nucleotide of interest.

In one embodiment, the polynucleotide further encodes a cytokine.

In one embodiment, the polynucleotide further encodes a selection marker.

In one embodiment, the polynucleotide further encodes a suicide gene.

In one embodiment, the polynucleotide further encodes CD20, or a modified version thereof.

In one embodiment, the polynucleotide further encodes thymidine kinase, or a modified version thereof.

In one embodiment, the polynucleotide further encodes human epidermal growth factor, or a modified version thereof.

In one embodiment, the polynucleotide further encodes a functionally inert truncated version of human epidermal growth factor receptor (EGFRt) or an enhanced version of EGFRt (eEGFRt).

In one embodiment, the polynucleotide further encodes caspase.

In another embodiment, the polynucleotide further encodes a glycosidase.

In one embodiment, the glycosidase comprises a sequence of any one or more of SEQ ID NO: 66-69, or a fragment or variant thereof.

In one embodiment, the glycosidase consists of the sequence of any one of SEQ ID NO: 66-69, or a fragment or variant thereof.

In one embodiment, the glycosidase comprises or consists of a sequence of any one of SEQ ID NO: 66-69, or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one aspect, there is provided a vector comprising the polynucleotide according to invention.

In one embodiment, the vector is a viral vector.

In one embodiment, the vector is a lentiviral vector.

In one embodiment, the vector is a bidirectional vector, such as a bidirectional lentiviral vector.

In one embodiment, the vector is an adeno-associated viral (AAV) vector.

In one embodiment, the vector is in the form of a nanoparticle.

In one embodiment, the polynucleotide or vector comprises one or more promoter(s), operably linked to the nucleotide sequence encoding the CAR or scFv.

In one embodiment, the polynucleotide or vector comprises a human phosphoglycerate kinase (PGK) promoter.

In one embodiment, the polynucleotide or vector comprises a human phosphoglycerate kinase (PGK) promoter operably linked to the nucleotide sequence encoding the CAR.

In one embodiment, the polynucleotide or vector comprises one or more nucleotide sequence encoding a suicide gene.

In one embodiment, the suicide gene is CD20. In one embodiment, the suicide gene is thymidine kinase (TK), or a modified version thereof. Preferably, the TK or modified version thereof is TK mut2.

In one embodiment, the polynucleotide or vector comprises one or more nucleotide sequence encoding a selection marker.

In one embodiment, the selection marker is CD20. In one embodiment, the selection marker is thymidine kinase (TK), or a modified version thereof.

In one embodiment, the polynucleotide or vector comprises one or more promoter(s), operably linked to the nucleotide sequence encoding CD20. In one embodiment, the polynucleotide or vector comprises one or more promoter(s) operably linked to the nucleotide sequence encoding thymidine kinase (TK), or a modified version thereof.

In one embodiment, the polynucleotide or vector comprises a human phosphoglycerate kinase (PGK) promoter. In one embodiment, the polynucleotide or vector comprises a human phosphoglycerate kinase (PGK) promoter operably linked to the nucleotide sequence encoding the suicide gene or the selection marker.

In one embodiment, the polynucleotide or vector comprises a human phosphoglycerate kinase (PGK) promoter, operably linked to the nucleotide sequence encoding CD20. In one embodiment, the polynucleotide or vector comprises a human phosphoglycerate kinase (PGK) promoter operably linked to the nucleotide sequence encoding thymidine kinase (TK), or a modified version thereof.

In one embodiment, the polynucleotide or vector comprises a minimal core promoter derived from cytomegalovirus (minCMV). In one embodiment, the polynucleotide or vector comprises a minimal core promoter derived from cytomegalovirus (minCMV) operably linked to the nucleotide sequence encoding the suicide gene or the selection marker.

In one embodiment, the polynucleotide or vector comprises a minimal core promoter derived from cytomegalovirus (minCMV), operably linked to the nucleotide sequence encoding CD20. In one embodiment, the polynucleotide or vector comprises a minimal core promoter derived from cytomegalovirus (minCMV), operably linked to the nucleotide sequence encoding thymidine kinase (TK), or a modified version thereof.

In one embodiment, the nucleotide sequence encoding the suicide gene or selection marker is encoded in an antisense orientation relative to the nucleotide sequence encoding the CAR.

In one embodiment, the polynucleotide or vector comprises a nucleotide sequence encoding the CAR and nucleotide sequence encoding a suicide gene or a selection marker.

In one embodiment, the polynucleotide or vector comprises a human phosphoglycerate kinase (PGK) promoter operably linked to the nucleotide sequence encoding the CAR and a minimal core promoter derived from cytomegalovirus (minCMV) operably linked to the nucleotide sequence encoding a suicide gene or a selection marker.

In one embodiment, the polynucleotide or vector comprises a nucleotide sequence encoding the CAR and nucleotide sequence encoding CD20. In one embodiment, the polynucleotide or vector comprises a nucleotide sequence encoding the CAR and nucleotide sequence encoding thymidine kinase (TK), or a modified version thereof.

In one embodiment, the polynucleotide or vector comprises a human phosphoglycerate kinase (PGK) promoter operably linked to the nucleotide sequence encoding the CAR and a minimal core promoter derived from cytomegalovirus (minCMV), operably linked to the nucleotide sequence encoding CD20. In one embodiment, the polynucleotide or vector comprises a human phosphoglycerate kinase (PGK) promoter operably linked to the nucleotide sequence encoding the CAR and a minimal core promoter derived from cytomegalovirus (minCMV), operably linked to the nucleotide sequence encoding thymidine kinase (TK), or a modified version thereof. In a preferred embodiment, the nucleotide sequence encoding the thymidine kinase (TK), or a modified version thereof is encoded in an antisense orientation relative to the nucleotide sequence encoding the CAR.

In one embodiment, the nucleotide sequence encoding the suicide gene or selection marker is encoded in an antisense orientation and the nucleotide sequence encoding the CAR is in a sense orientation. In one embodiment, the nucleotide sequence encoding the suicide gene or selection marker is encoded in a sense orientation and the nucleotide sequence encoding the CAR is in an antisense orientation.

In one aspect, there is provided a cell comprising the CAR, the scFv, the polynucleotide, or the vector according to the invention.

In another aspect, there is provided a cell comprising the polynucleotide, or the vector according to the invention.

In a further aspect, there is provided a cell comprising the CAR, or the scFv according to the invention.

In one embodiment, the cell is a eukaryotic cell, such as a mammalian cell. In one embodiment, the cell is selected from the group consisting of a rodent cell, such as a mouse or rat cell, a feline cell, a canine cell, and a human cell.

In a preferred embodiment, the cell is a human cell.

In one embodiment the cell is an immune cell.

In one embodiment, the cell is selected from the group consisting of a T cell, Natural Killer (NK) cell, invariant-NK T cell, Cytokine-Induced Killer (CIK) cell, and macrophage, optionally wherein the cell is an autologous or allogeneic cell.

In one embodiment, the cell is a T cell.

In one embodiment, the cell is an invariant-NK T cell.

In one embodiment, the cell is a Cytokine-Induced Killer (CIK) cell.

In one embodiment, the cell is a macrophage.

In one embodiment, the cell is an autologous cell.

In one embodiment, the cell is an allogeneic cell.

In one aspect, there is provided a composition comprising the CAR, the scFv, the polynucleotide, the vector, or the cell according to the invention.

In one aspect, there is provided a pharmaceutical composition comprising the CAR, the scFv, the polynucleotide, the vector, or the cell according to the invention.

In one aspect, there is provided the CAR, the scFv, the polynucleotide, the vector, the cell, the composition, or the pharmaceutical composition, according to the invention for use in therapy.

In one aspect, there is provided the CAR, the scFv, the polynucleotide, the vector, the cell, the composition, or the pharmaceutical composition, according to the invention for use in treatment of cancer.

In one embodiment, there is provided the CAR, the scFv, the polynucleotide, the vector, the cell, the composition, or the pharmaceutical composition, according to the invention for use in the treatment of cancer, wherein:
a) the cancer is primary cancer, optionally a gastrointestinal cancer, a colorectal cancer, a pancreatic cancer and/or a stomach cancer;
b) the cancer is a secondary cancer, optionally a liver metastasis, optionally a liver metastasis of a colorectal cancer, and/or a liver metastasis of a pancreatic ductal adenocarcinoma (PDAC); and/or
c) the cancer is a neuroendocrine tumor.

In one embodiment, the cancer is primary cancer.

In one embodiment, the cancer is a gastrointestinal cancer, a colorectal cancer, a pancreatic cancer and/or a stomach cancer.

In one embodiment, the cancer is a gastrointestinal cancer.

In one embodiment, the cancer is a colorectal cancer.

In one embodiment, the cancer is a pancreatic cancer. In one embodiment, the cancer is a pancreatic ductal adenocarcinoma (PDAC). In one embodiment, the cancer is a pancreatic neuroendocrine tumor.

In one embodiment, the cancer is a stomach cancer.

In one embodiment, the cancer is a secondary cancer.

In one embodiment, the cancer is a liver metastasis.

In one embodiment, the cancer is a liver metastasis of a colorectal cancer.

In one embodiment, the cancer is a liver metastasis of a pancreatic ductal adenocarcinoma (PDAC).

In one embodiment, the cancer is a neuroendocrine tumor.

In one aspect, there is provided a use of the scFv of the invention for determining the level of cadherin-17 (CDH-17) in a sample.

In one embodiment, there is provided the use of the scFv of the invention for determining the level of cadherin-17 (CDH-17) in a sample, wherein the sample is from a subject.

In one embodiment, the subject is a human subject.

In another embodiment, the subject is a human subject with cancer.

In one aspect, there is provided a method for identifying a subject suitable for treatment with an anti-CDH-17 therapy, wherein the method comprises determining a CDH-17 expression level in a sample isolated from the subject, wherein the CDH-17 expression level is determined using the scFv of the invention.

In one embodiment, the subject is a human subject.

### DESCRIPTION OF THE DRAWINGS

FIG. 1. Target discovery strategy.

Schematic representation of steps undertaken in the rational search for candidate CAR targets.

FIG. 2. Identification of target antigens.

Heatmap representing the expression of known (A) and unknown (B) target antigens in liver metastasis samples retrieved from patients with colorectal cancer. Data derive from the RNA sequencing analysis of samples including more than 70% of tumor cells. After obtaining the expression matrix in TPM (Transcripts Per Kilobase Million), values were converted in deciles and highly-expressed genes were defined as those belonging in the first three deciles (10, 9 and 8). (C) Expression of the 30 top-ranking antigens in healthy tissues. Starting from a global matrix including mRNA expression data of every gene in all available tissue, means and standard deviations of all genes in each tissue were calculated to identify four expression classes: high, mid, low, and not detected. (D) Expression (transcripts per million) of CDH-17 in tumors (red dots) and adjacent normal tissues (green dots) from different patients with cancer, plotted using The Cancer Genome Atlas (TCGA) data. Highlighting of tumor types in red indicates higher expression in tumor versus normal tissue. Right: focus on expression data in CRC at different stages.

FIG. 3. Schematic representation of exemplary CAR constructs.

Schematic representation of bidirectional lentiviral constructs encoding the CDH-17-specific CARs and the CD20 marker gene. In order from left to right each construct comprises boxes, which represent the following domains/regions: a selection marker/suicide gene (CD20); a mCMV promoter or ‘minCMV’; a human PGK promoter; a single-chain fragment variable region (e.g., Lic3 or A4_4R); an extracellular spacer or linker (LNGFR spacer (NMS) or ‘H’ [from IgG1]); a co-stimulatory domain (e.g., CD28); and a signaling domain (e.g., CD3-zeta). A transmembrane isn't explicitly shown, however, it may comprise a sequence derived from the co-stimulatory domain or, alternatively, the linker/spacer sequence.

FIG. 4. Assessment of CAR T cell production.

Peripheral blood-derived human T cells were activated with beads, transduced with lentiviral vectors, and expanded in IL-7 and IL-15. CAR T cell products were analyzed at the end of manufacturing. (A) T-cell fold expansion (n=5 donors). (B) Frequency of CD4 and CD8 T cells. (C) Frequency of memory subset according to CD45RA and CD62L expression. (D) Frequency of HLA-DR expressing T cells. (E) Frequency of PD-1 expressing T cells. T_{SCM}: stem memory T cells. T_{CM}: central memory T cells; T_{EM}: effector memory T cells; T_{EMRA}: effector memory RA+ T cells.

FIG. 5. In vitro CAR T cell mediated tumor cell killing.

(A) Killing of LoVo and BxPC3 tumor cells was quantified after co-culture with CDH17.28z CAR T cells at different E:T ratios (n=4 donors). Killing is expressed as elimination index, which was calculated as compared to untransduced T cells. (B) Tumor cell killing comparing constructs with different spacer regions (LNGFR spacer (NMS) vs hinge) or scFv (Lic3 vs A4_4R). P values (**P<0.01; ***P<0.001; ****P<0.0001) were determined by two-way ANOVA (A) or paired t test (B). Data are presented as means+/−SEM.

FIG. 6. In vivo CAR T cell mediated tumor cell killing.

(A) Immunodeficient NSG mice were intraperitoneally infused with LoVo cells marked with a secreted luciferase and, after 10 days, treated with A4_4R-NMS CAR T cells, A4_4R.hinge CAR T cells or untransduced T cells (6×10⁶ cells/mouse). Tumor growth was followed by measuring bioluminescence signal in peripheral blood. (B) Left: tumor growth kinetics. Middle: tumor levels at day 6 after T-cell infusion. Right: tumor levels at day 25 after T-cell infusion. P values (*P<0.05; **P<0.01) were determined by two-way ANOVA. Data are presented as means+/−SEM.

FIG. 7. Assessment of CAR T cell production.

A. Schematics of the bidirectional lentiviral constructs including the CDH17-specific CARs and the CD20 marker gene. Red box: single-chain fragment variable. Orange box: extracellular spacer. Violet box: costimulatory endodomain. Green box: activatory endodomain. Peripheral blood-derived human T cells were activated with beads, transduced with lentiviral vectors and expanded in IL-7 and IL-15. CAR T cell products were analyzed at the end of manufacturing. B. T-cell fold expansion (n=10 donors for Ut, IgG1 h and NWL conditions; n=3 donors for NMS condition). C. Frequency of NGFR-spaced CAR expressing T cells. D. Frequency of CD20 expressing T cells. E. Frequency of CD4 and CD8 T cells. F. Frequency of memory subset according to CD45RA and CD62L expression. T_{SCM}: stem memory T cells. T_{CM}: central memory T cells; T_{EM}: effector memory T cells; T_{EMRA}: effector memory RA+ T cells. P value (****P<0.0001) was determined by one-way ANOVA (B). Data are presented as means+/−SEM.

FIG. 8. CDH17 immunohistochemistry stainings of human tissues.

Representative stainings of primary colorectal carcinoma (A), liver metastasis from colorectal carcinoma (B), primary pancreatic ductal adenocarcinoma (C), and pancreatic neuroendocrine tumor (D) with the commercial anti-CDH17 antibody (Abcam, Clone EPR 3996). Experiments were performed in multiple primary samples with similar results.

FIG. 9. Generation of CDH17 truncated mutants and identification of the extracellular cadherin containing the A4_4R CAR epitope.

Schematics of CDH17 truncated mutants. SP, signal peptide; EC, extracellular cadherin; TM, transmembrane; IC, intracellular domain. B. CDH17^{neg}SW620 colorectal cancer cells were transduced with bidirectional lentiviral vectors expressing WT CDH17 or truncated mutants and NGFR as marker gene, followed by flow cytometry analysis with NGFR (left) and the commercial anti-CDH17 antibody (Santa Cruz, Clone H1-1), which binds to EC7 (right). C. Killing of SW620 cells quantified after coculture with CDH17.28z cells having as extracellular spacer IgG1 hinge (left), NMS (center) and NWL (right) at 1:5 effector-to-target ratio (n=3 donors). Killing is expressed as elimination index, which was calculated as compared to untransduced T cells. P values (**P<0.01; ***P<0.001; ****P<0.0001) were determined by one-way ANOVA (A) or paired t test (B). Data are presented as means+/−SEM.

FIG. 10. In vivo efficacy of CDH17 CAR T cells in a subcutaneous model of CRC.

Anti-tumor efficacy of CDH17.28z cells in xenograft mouse model of colorectal cancer. Immunodeficient NSG mice were subcutaneously infused with LoVo cells marked with a secreted luciferase and treated with CDH17.28z_H CAR T cells (n=8), CDH17.28z_NMS CAR T cells (n=8), CDH17.28z_NWL CAR T cells (n=7) or untransduced T cells (n=4) (10×10⁶ cells/mouse). Tumor growth was followed by measuring bioluminescence signal in peripheral blood. A. Kinetics of tumor growth. RLU, relative light unit. B. Tumor-related Kaplan-Meier survival rates. C, D. Expansion (C) and HLA-DR expression at day 8 (RFI, relative fluorescence intensity, D) of CAR T cells after infusion in peripheral blood. E, F. CD20 expression (percentage of positive cells, E) and NGFR expression (percentage of positive cells, F) in tumor infiltrating lymphocytes at sacrifice. P values (*P<0.05; ***P<0.001; ****P<0.0001) were determined by log-rank Mantel-Cox test (B) or by one-way ANOVA (D) or by two-tailed t-test (F). Data are presented as means+/−SEM.

FIG. 11. In vivo efficacy of CDH17 CAR T cells in a subcutaneous model of PDAC.

Anti-tumor efficacy of CDH17.28z cells in xenograft mouse model of pancreatic adenocarcinoma. Immunodeficient NSG mice were subcutaneously infused with AsPC-1 cells marked with a secreted luciferase and treated with CDH17.28z_H CAR T cells (n=8), CDH17.28z_NMS CAR T cells (n=8), CDH17.28z_NWL CAR T cells (n=7) or untransduced T cells (n=3) (10×10⁶ cells/mouse). Tumor growth was followed by measuring bioluminescence signal in peripheral blood. A. Kinetics of tumor growth. RLU, relative light unit. B. Tumor-related Kaplan-Meier survival rates. C, D. Expansion (C) and HLA-DR expression (D) at day 9 (RFI, relative fluorescence intensity) of CAR T cells after infusion in peripheral blood. P values (*P<0.05; **P<0.01; ***P<0.001; ****P<0.0001) were determined by log-rank Mantel-Cox test (B) or by one-way ANOVA (D). Data are presented as means+/−SEM.

FIG. 12. Cytokine production of CDH17 CAR T cells in vitro.

Production of IFN-γ (A) and TNF-α (B) after 24 h coculture of CDH17.28z_H or CDH17.28z_NWL cells with LoVo colorectal cells at 1:10 effector-to-target ratio (n=3 donors in technical duplicates). UT, untransduced control T cells. P value (*P<0.05) was determined by one-way ANOVA. Data are presented as means+/−SEM.

FIG. 13. A. In vitro efficacy of CEA and CDH17 CAR T cells.

Schematics of the bidirectional lentiviral constructs including the CEA or CDH17 CAR and the CD20 marker gene. CEA CAR was generated as the CDH17.28z_H construct and contains a specific scFv derived from the BW431-26 mAb. Peripheral blood-derived human T cells were activated with beads, transduced with lentiviral vectors and expanded in IL-7 and IL-15. CAR T cell products were analyzed at the end of manufacturing. Ut denote untransduced control T cells. B. T-cell fold expansion (n=3 donors). C. Frequency of NGFR expressing T cells. D. Frequency of CD4 and CD8 T cells. E. Killing of LoVo cells quantified after coculture with CDH17.28z or CEA.28z cells at multiple effector-to-target ratios (n=3-6 donors). Killing is expressed as elimination index, which was calculated as compared to untransduced T cells. F. Production of IFN-γ and TNF-α after 24 h coculture of CDH17.28z or CEA.28z cells with LoVo colorectal cells at 1:10 E:T ratio (n=3 donors in technical duplicates). UT, untransduced control T cells. (**P<0.01; ***P<0.001) were determined by paired t-test (C) and two-way ANOVA (E). Data are presented as means+/−SEM.

FIG. 14. In vitro efficacy of CDH17 CAR T cells against patient-derived organoids from CRC-LM.

Representative bright-field microscopy images (left) and quantification (right) of killing of organoids derived from patients with colorectal carcinoma metastatic to the liver after coculture with CDH17.28z CAR T cells from healthy donors at 1:1 E:T ratio (n=1 in technical triplicate). P values (*P<0.05) were determined by paired t-test. Data are presented as means+/−SEM.

FIG. 15. Manufacturing of CAR T cells from patients with liver metastasis from colorectal carcinoma.

Peripheral blood derived human T cells were activated with beads, transduced with lentiviral vectors and expanded in IL-7 and IL-15. CAR T cell products were analyzed at the end of manufacturing. A. T-cell fold expansion (n=3-7 donors). B. Frequency (left) and intensity (right) of CD20 expression by T cells. RFI, relative fluorescence intensity. C. Frequency of NGFR expressing T cells. D. Frequency of CD4 and CD8 T cells. E. Frequency of memory subset according to CD45RA and CD62L expression. T_{SCM}: stem memory T cells. T_{CM}: central memory T cells; T_{EM}: effector memory T cells; T_{EMRA}: effector memory RA+ T cells. P values (*P<0.05; **P<0.01; ***P<0.001) were determined by one-way ANOVA (B, C). Data are presented as means+/−SEM.

FIG. 16. Manufacturing of CAR T cells from patients with liver metastasis from pancreatic ductal adenocarcinoma.

Peripheral blood derived human T cells were activated with beads, transduced with lentiviral vectors and expanded in IL-7 and IL-15. CAR T cell products were analyzed at the end of manufacturing. A. T-cell fold expansion (n=3 donors). B. Frequency (left) and intensity (right) of CD20 expression by T cells. RFI, relative fluorescence intensity. C. Frequency of NGFR expressing T cells. D. Frequency of CD4 and CD8 T cells. E. Frequency of memory subset according to CD45RA and CD62L expression. T_{SCM}: stem memory T cells. T_{CM}: central memory T cells; T_{EM}: effector memory T cells; T_{EMRA}: effector memory RA+ T cells. P value (**P<0.01) was determined by one-way ANOVA. Data are presented as means+/−SEM.

FIG. 17. Manufacturing of CAR T cells from patients with primary pancreatic ductal adenocarcinoma.

Peripheral blood derived human T cells were activated with beads, transduced with lentiviral vectors and expanded in IL-7 and IL-15. CAR T cell products were analyzed at the end of manufacturing. A. T-cell fold expansion (n=2 donors). B. Frequency (left) and intensity (right) of CD20 expression by T cells. RFI, relative fluorescence intensity. C. Frequency of NGFR expressing T cells. D. Frequency of CD4 and CD8 T cells. E. Frequency of memory subset according to CD45RA and CD62L expression. T_{SCM}: stem memory T cells. T_{CM}: central memory T cells; T_{EM}: effector memory T cells; T_{EMRA}: effector memory RA+ T cells. P value (*P<0.05) was determined by one-way ANOVA. Data are presented as means+/−SEM.

FIG. 18. In vitro efficacy of patient-derived CDH17 CAR T cells from CRC-LM against patient-derived organoids from CRC-LM.

Killing of patient-derived organoids (PDOs) marked with luciferase after coculture with CDH17.28z CAR T cells from patients with liver metastasis from colorectal carcinoma at 1:1 E:T ratio. PDOs were detected by measuring bioluminescence signal. A, B. Representative bright-field microscopy images (A) and quantification of PDO killing (B) by autologous CDH17.28z_H (left) and CDH17.28z_NWL (right) CAR T cells (n=1 in technical duplicate). C-F. Representative bright-field microscopy images (C, E) and quantification of PDOs killing by allogeneic CDH17.28z_H CAR T cells (D left, F left) and by allogeneic CDH17.28z_NWL CAR T cells (D right, F right). RLU, relative light unit. P values (*P<0.05; **P<0.01) were determined by paired t-test. Data are presented as means+/−SEM.

FIG. 19. Immunofluorescence staining of CDH17 localization in primary healthy colon and tumor tissues.

Representative confocal images of healthy (A) and tumoral (B) colon and liver metastasis from colorectal carcinoma (C) stained for CDH17 (green) and Occludin (red, tight junctions). Zoomed images of magnification 63×.

FIG. 20. Validation of CDH17.28z CAR-T cells administration route in xenograft mouse model of colorectal cancer.

Immunodeficient NSG mice were intrahepatic infused with LoVo cells marked with a secreted luciferase and treated either intraliver with CDH17.28z_H CAR T cells (n=5), CDH17.28z_NWL CAR T cells (n=5) or intravenous with CDH17.28z_H CAR T cells (n=5), CDH17.28z_NWL CAR T cells (n=5) (10×10⁶ cells/mouse). Untransduced T cells (n=4) were administered as negative control. Tumor growth was followed by measuring bioluminescence signal in peripheral blood. A. Kinetics of tumor growth. RLU, relative light unit. B. Expansion of CDH17.28z_H (left) and CDH17.28z_NWL (right) CAR T cells after infusion in peripheral blood. Data are presented as means+/−SEM.

FIG. 21. Validation of CDH17.28z CAR-T cells administration route in HSPC-humanized SGM3 mouse model of colorectal cancer.

NSG mice transgenic for the expression of human SCF, GM-CSF and IL-3 (SGM3) were infused with human cord blood derived hematopoietic stem and progenitor cells (CB HSPCs), intrahepatic LoVo cells marked with a secreted luciferase and treated either intraliver with CDH17.28z_H CAR T cells (n=7), CDH17.28z_NWL CAR T cells (n=7) or intravenous with CDH17.28z_H CAR T cells (n=7), CDH17.28z_NWL CAR T cells (n=7) (5×10⁶ cells/mouse). Untransduced T cells (n=4) were administered as negative control. Tumor growth was followed by measuring bioluminescence signal in peripheral blood. A. Kinetics of tumor growth. RLU, relative light unit. B. Tumor levels (left), HLA-DR expression (percentage of positive cells) (middle) and T cells in peripheral blood (right) at day 7 after CDH17.28z_H CAR T cells infusion. C. Tumor levels (left), HLA-DR expression (percentage of positive cells) (middle) and T cells in peripheral blood (right) at day 7 after CDH17.28z_NWL CAR T cells infusion. P values (*P<0.05; **P<0.01) were determined by two-tailed t-test. Data are presented as means+/−SEM.

FIG. 22. Assessing the potential systemic toxicity of CDH17.28z CAR-T cells in HSPC-humanized SGM3 mouse model of colorectal cancer.

A-C. Percentage of weight loss (A), concentration of circulating myeloid-derived cytokines (B), and weight loss related Kaplan-Meier survival rates (C) of mice from FIG. 20 treated either intraliver or intravenous with CDH17.28z_H CAR T cells. D-F. Percentage of weight loss (D), concentration of circulating myeloid-derived cytokines (E), and weight loss related Kaplan-Meier survival rates (F) of mice from FIG. 20 treated either intraliver or intravenous with CDH17.28z_NWL CAR T cells. P values (*P<0.05; **P<0.01; ***P<0.001) were determined by two-tailed t-test (B) or by log-rank Mantel-Cox test (C, F). Data are presented as means+/−SEM.

FIG. 23. Validation of CDH17.28z CAR-T cells administration route in xenograft mouse model of pancreatic adenocarcinoma.

Immunodeficient NSG mice were intrapancreatic infused with AsPC-1 cells marked with a secreted luciferase and treated either intrapancreatic with CDH17.28z_H CAR T cells (n=5), CDH17.28z_NWL CAR T cells (n=5) or intravenous with CDH17.28z_H CAR T cells (n=3), CDH17.28z_NWL CAR T cells (n=3) (10×10⁶ cells/mouse). Untransduced T cells (n=3) were administered as negative control. Tumor growth was followed by measuring bioluminescence signal in peripheral blood. A. Kinetics of tumor growth. RLU, relative light unit. B. Tumor-related Kaplan-Meier survival rates of CDH17.28z_H (left) and CDH17.28z_NWL (right) CAR T cell treated mice. P value was determined by log-rank Mantel-Cox test (B).

FIG. 24. Generation and in vitro efficacy of third generation CDH17 CAR-T cells.

A. Schematics of the bidirectional lentiviral constructs including the CDH17-specific CARs and the CD20 marker gene. Red denotes the second generation CAR having the A4-4R scFv; blue denotes the third generation CAR having the A4-4R scFv; green denotes third generation CAR having the VHH1 nanobody. Peripheral blood-derived human T cells were activated with beads, transduced with lentiviral vectors and expanded in IL-7 and IL-15. B. T-cell fold expansion (n=3 donors). C. Frequency of CD20 expressing T cells at day 9 of manufacturing. D. Frequency of NGFR expressing T cells at day 9 of manufacturing. E. Frequency of CD4 and CD8 T cells at day 9 of manufacturing. F-H. Killing of LoVo cells quantified after coculture with CAR T cells (n=6 donors). F. Killing at multiple effector-to-target ratios. Killing is expressed as elimination index, which was calculated as compared to untransduced T cells. G, I. Killing of LoVo cells at 1:25 E:T ratio. H. Production of IFN-γ (left) and TNF-α (right) after 24 h coculture of LoVo cells with CAR T cells at 1:10 E:T ratio. P value (*P<0.05; **P<0.01) were determined by paired t-tes (G, I) and one-way ANOVA (H). Data are presented as means+/−SEM.

FIG. 25. Manufacturing of third generation anti-CDH17 CAR T cells from patients with liver metastasis from colorectal carcinoma.

Peripheral blood derived-human T cells were activated with beads, transduced with lentiviral vectors and expanded in IL-7 and IL-15. CAR T cell products were analyzed at the end of manufacturing. A. T-cell fold expansion (n=3 donors). B. Frequency of CD20 expressing T cells. C. Frequency of CD4 and CD8 T cells. D. Frequency of memory subset according to CD45RA and CD62L expression. T_{SCM}: stem memory T cells. T_{CM}: central memory T cells; EM: effector memory T cells; T_{EMRA}: effector memory RA+ T cells. P value (**P<0.01) was determined by unpaired t-test (B). Data are presented as means+/−SEM.

FIG. 26. Generation of CAR-T cells expressing the thymidine kinase (TK) suicide gene.

A-C. The TK suicide gene was cloned under the control of the mCMV promoter in CAR backbones expressing the CDH17.28z_H (A), the CDH17.28z_NWL (B) and the CDH17.28BBz_H (C) CAR.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

It will be understood that when referring to a protein or polypeptide herein, the same may equally be applied to a polynucleotide encoding the same, and, where relevant (i.e., when referring to a coding sequence within a polynucleotide) vice versa.

### Cadherin-17 (CDH-17)

Cadherin-17 (CDH-17) is a cell-to-cell adhesion glycoprotein that is overexpressed in several cancers of the gastrointestinal tract, including pancreas, colorectum, stomach, liver and esophagus, and in neuroendocrine tumors. Upon interaction with the α2β1 integrin, CDH17 promotes tumor cell proliferation, adhesion, and metastatic colonization of the liver.

### Chimeric Antigen Receptors (CAR)

“Chimeric antigen receptor” or “CAR” or “CARs” as used herein refers to engineered receptors which can confer an antigen specificity onto cells (for example T cells such as naive T cells, central memory T cells, effector memory T cells or combinations thereof). CARs are also known as artificial T-cell receptors, chimeric T-cell receptors or chimeric immunoreceptors.

CARs are commonly classified into ‘generations’, by virtue of the composition of their intracellular signalling domain(s). All CARs typically comprise an extracellular antigen-binding domain, normally an scFv, joined to a membrane-anchoring transmembrane domain by a linker or spacer sequence. Whilst first generation CARs comprise a single intracellular signalling domain that is typically a single CD3 zeta chain, second and third generation CARs additionally comprise one or two further co-stimulatory domains (respectively), typically CD28, 4-1BB, and/or OX-40. Fourth generation CARs are structurally similar to second generation CARs, however, are typically provided alongside an expression cassette (e.g., in a CAR T cell) that encodes an additional transgene such as a cytokine.

In one embodiment the CAR of the invention is a first generation CAR.

In one embodiment the CAR of the invention is a second generation CAR.

In one embodiment the CAR of the invention is a third generation CAR.

In one embodiment the CAR of the invention is a fourth generation CAR.

Preferably the CARs of the invention comprise an antigen-specific targeting region, which may comprise an scFv, an extracellular domain such as a linker or hinge, a transmembrane domain, optionally one or more co-stimulatory domains, and an intracellular signaling domain.

In some embodiments, the CARs of the invention are provided alongside one or more nucleotides of interest or transgenes. In some embodiments, the transgene encodes a cytokine.

### Antigen-Specific Targeting Domain

The antigen-specific targeting domain (or antigen-binding domain) provides the CAR with the ability to bind to the target antigen of interest. The antigen-specific targeting domain preferably targets an antigen of clinical interest against which it would be desirable to trigger an effector immune response that results in cell killing.

The antigen-specific targeting domain may be any protein or peptide that possesses the ability to specifically recognize and bind to a biological molecule (e.g., a cell surface receptor or tumor protein, or a component thereof). The antigen-specific targeting domain includes any naturally occurring, synthetic, semi-synthetic, or recombinantly produced binding partner for a biological molecule of interest.

Illustrative antigen-specific targeting domains include antibodies or antibody fragments or derivatives, extracellular domains of receptors, ligands for cell surface molecules/receptors, or receptor binding domains thereof, and tumor binding proteins.

In a preferred embodiment, the antigen-specific targeting domain is, or is derived from, an antibody. An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain variable fragment (scFv), a heavy chain variable region (VH), a light chain variable region (VL) and a camelid antibody (VHH).

In a preferred embodiment, the binding domain is a single chain variable fragment (scFv). The scFv may be, for example, a murine, human or humanized scFv.

“Complementarity determining region” or “CDR” with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs.

“Heavy chain variable region” or “VH” refers to the fragment of the heavy chain of an antibody that contains three CDRs interposed between flanking stretches known as framework regions, which are more highly conserved than the CDRs and form a scaffold to support the CDRs.

“Light chain variable region” or “VL” refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence.

With respect to targeting domains that target cancer antigens, the selection of the targeting domain will depend on the type of cancer to be treated, and may target tumor antigens. A tumor sample from a subject may be characterized for the presence of certain biomarkers or cell surface markers.

A preferred target antigen of the present invention is CDH17.

A tumor antigen or cell surface molecule is selected that is found on the individual subject's tumor cells. Preferably the antigen-specific targeting domain targets a cell surface molecule that is found on tumor cells and is not substantially found on normal tissues.

Preferably, the antigen-binding domain specifically binds to a tumor antigen (e.g. CDH17). In one embodiment, the antigen-binding domain specifically binds CDH17.

In a preferred embodiment, the antigen-binding domain is a scFv.

In a preferred embodiment, the antigen-binding domain is a scFv with specificity for CDH17.

In one embodiment, the antigen-binding domain (e.g. scFv) specifically binds the EC1 domain of CDH17.

In one embodiment, the antigen-binding domain comprises:
a) heavy chain variable region (VH) complementarity determining regions (CDRs) with the sequences: CDR1—DHTIHWMR (SEQ ID NO: 1); CDR2—YIYPRDGITGYNERFRGK (SEQ ID NO: 2); and CDR3—WGYSYRNYAYYYDYWGQGTL (SEQ ID NO: 3); or variants thereof each having up to three amino acid substitutions, additions or deletions; and
b) light chain variable region (VL) CDRs with the sequences: CDR1—INCRSSQSLLHSSNQR (SEQ ID NO: 4); CDR2—PPKVLIYWASTRES (SEQ ID NO: 5); and CDR3—QQYYSYPWTFGQ (SEQ ID NO: 6); or variants thereof each having up to three amino acid substitutions, additions or deletions.

In one embodiment, the antigen-binding domain comprises:
a) a VH domain comprising the sequence of SEQ ID NO: 7; and
b) a VL domain comprising the sequence of SEQ ID NO: 8;
   or variants thereof, each having at least 75% sequence identity thereto.

Exemplary VH domain (SEQ ID NO: 7):
QVQLVQSGAEVKKPGASVKVSCKASGYTLTDHTIHWMRQAPGQRLEWIG
YIYPRDGITGYNERFRGKATLTADTSASTAYMELSSLRSEDTAVYYCAR
WGYSYRNYAYYYDYWGQGTLVTVSS
Exemplary VL domain (SEQ ID NO: 8):
DIVMTQSPDSLAVSLGERATINCRSSQSLLHSSNQRNYLAWYQQKPGQP
PKVLIYWASTRESGVPDRFSGSGSGTDFTLTISSLQAEDVAVYYCQQYY
SYPWTFGQGTKVEIK

In one embodiment, the antigen-binding domain comprises a sequence having at least 75% sequence identity to the sequence of SEQ ID NO: 9, such as at least 80%, at least 85%, at least 90%, at least 91%, at least 92%, at least 93%, at least 94%, at least 95%, at least 96%, at least 97%, at least 98%, at least 99%, or 100% sequence identity thereto.

Exemplary scFv (SEQ ID NO: 9):
MEAPAQLLFLLLLWLPDTTGDIVMTQSPDSLAVSLGERATINCRSSQSL
LHSSNQRNYLAWYQQKPGQPPKVLIYWASTRESGVPDRFSGSGSGTDFT
LTISSLQAEDVAVYYCQQYYSYPWTFGQGTKVEIKGGGGSGGGGSGGGG
SQVQLVQSGAEVKKPGASVKVSCKASGYTLTDHTIHWMRQAPGQRLEWI
GYIYPRDGITGYNERFRGKATLTADTSASTAYMELSSLRSEDTAVYYCA
RWGYSYRNYAYYYDYWGQGTLVTVSSSPV

In one embodiment, the antigen-binding domain is encoded by SEQ ID NO: 16, or a sequence with at least 75% sequence identity thereto.

Nucleotide sequence encoding an exemplary scFv
(SEQ ID NO: 16):
ATGGAGGCCCCCGCCCAGCTGCTGTTCCTGCTGCTGCTGTGGCTGCCCG
ACACCACCGGCGACATCGTGATGACCCAGAGCCCCGACAGCCTGGCCGT
GAGCCTGGGCGAGAGAGCCACCATCAACTGCAGAAGCAGCCAGAGCCTG
CTGCACAGCAGCAACCAGAGAAACTACCTGGCCTGGTACCAGCAGAAGC
CCGGCCAGCCCCCCAAGGTGCTGATCTACTGGGCCAGCACCAGAGAGAG
CGGCGTGCCCGACAGATTCAGCGGCAGCGGCAGCGGCACCGACTTCACC
CTGACCATCAGCAGCCTGCAGGCCGAGGACGTGGCCGTGTACTACTGCC
AGCAGTACTACAGCTACCCCTGGACCTTCGGCCAGGGCACCAAGGTGGA
GATCAAGGGCGGCGGCGGCAGCGGCGGCGGCGGCAGCGGCGGCGGCGGC
AGCCAGGTGCAGCTGGTGCAGAGCGGCGCCGAGGTGAAGAAGCCCGGCG
CCAGCGTGAAGGTGAGCTGCAAGGCCAGCGGCTACACCCTGACCGACCA
CACCATCCACTGGATGAGACAGGCCCCCGGCCAGAGACTGGAGTGGATC
GGCTACATCTACCCCAGAGACGGCATCACCGGCTACAACGAGAGATTCA
GAGGCAAGGCCACCCTGACCGCCGACACCAGCGCCAGCACCGCCTACAT
GGAGCTGAGCAGCCTGAGAAGCGAGGACACCGCCGTGTACTACTGCGCC
AGATGGGGCTACAGCTACAGAAACTACGCCTACTACTACGACTACTGGG
GCCAGGGCACCCTGGTGACCGTGAGCAGCTC

In one aspect, there is provided a chimeric antigen receptor (CAR) comprising an antigen-binding domain, wherein the antigen-binding domain comprises:
a) heavy chain variable region (VH) complementarity determining regions (CDRs) with the sequences: CDR1—DYYMY (SEQ ID NO: 42); CDR2—SISFDGTYTYYTDRVKG (SEQ ID NO: 43); and CDR3—DRPAWFPY (SEQ ID NO: 44); or variants thereof each having up to three amino acid substitutions, additions or deletions; and
b) light chain variable region (VL) CDRs with the sequences: CDR1—RSSQSIVHSNGNTYLE (SEQ ID NO: 39); CDR2—KVSNRFS (SEQ ID NO: 40); and CDR3—FQGSHVPLT (SEQ ID NO: 41); or variants thereof each having up to three amino acid substitutions, additions or deletions.

In one embodiment, the antigen-binding domain comprises:
a) a VH domain comprising the sequence of SEQ ID NO: 58; and
b) a VL domain comprising the sequence of SEQ ID NO: 59;
   or variants thereof, each having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

Exemplary VH domain (SEQ ID NO: 58):
QVQLVESGGGVVQPGRSLRLSCAASGFTFSDYYMYWVRQAPGKGLEWVA
SISFDGTYTYYTDRVKGRFTISRDNSKNTLYLQMNSLRAEDTAVYYCAR
DRPAWFPYWGQGTLVTVSA
Exemplary VL domain (SEQ ID NO: 59):
DIVMTQTPLSLSVTPGQPASISCRSSQSIVHSNGNTYLEWYLQKPGQSP
QLLIYKVSNRFSGVPDRFSGSGSGTDFTLKISRVEAEDVGVYYCFQGSH
VPLTFGAGTKLELKGAP

In one embodiment, the antigen-binding domain comprises a sequence having at least 90% sequence identity to the sequence of SEQ ID NO: 45.

In one embodiment, the antigen-binding domain comprises a sequence having at least 75% sequence identity to SEQ ID NO: 45, such as at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 45.

Exemplary scFv (SEQ ID NO: 45):
MEAPAQLLFLLLLWLPDTTGQVQLVESGGGVVQPGRSLRLSCAASGFTF
SDYYMYWVRQAPGKGLEWVASISFDGTYTYYTDRVKGRFTISRDNSKNT
LYLQMNSLRAEDTAVYYCARDRPAWFPYWGQGTLVTVSAGGGGSGGGGS
GGGGSGDIVMTQTPLSLSVTPGQPASISCRSSQSIVHSNGNTYLEWYLQ
KPGQSPQLLIYKVSNRFSGVPDRFSGSGSGTDFTLKISRVEAEDVGVYY
CFQGSHVPLTFGAGTKLELKGAP

In one embodiment, the antigen-binding domain is encoded by SEQ ID NO: 46, or a sequence with at least 75% sequence identity thereto.

Nucleotide sequence encoding an exemplary scFv
(SEQ ID NO: 46):
ATGGAGGCCCCCGCCCAGCTGCTGTTCCTGCTGCTGCTGTGGCTGCCCG
ACACCACCGGCCAGGTGCAGCTGGTGGAGAGCGGCGGCGGCGTGGTGCA
GCCCGGCAGAAGCCTGAGACTGAGCTGCGCCGCCAGCGGCTTCACCTTC
AGCGACTACTACATGTACTGGGTGAGACAGGCCCCCGGCAAGGGCCTGG
AGTGGGTGGCCAGCATCAGCTTCGACGGCACCTACACCTACTACACCGA
CAGAGTGAAGGGCAGATTCACCATCAGCAGAGACAACAGCAAGAACACC
CTGTACCTGCAGATGAACAGCCTGAGAGCCGAGGACACCGCCGTGTACT
ACTGCGCCAGAGACAGACCCGCCTGGTTCCCCTACTGGGGCCAGGGCAC
CCTGGTGACCGTGAGCGCCGGCGGCGGCGGCAGCGGCGGCGGCGGCAGC
GGCGGCGGCGGCAGCGGCGACATCGTGATGACCCAGACCCCCCTGAGCC
TGAGCGTGACCCCCGGCCAGCCCGCCAGCATCAGCTGCAGAAGCAGCCA
GAGCATCGTGCACAGCAACGGCAACACCTACCTGGAGTGGTACCTGCAG
AAGCCCGGCCAGAGCCCCCAGCTGCTGATCTACAAGGTGAGCAACAGAT
TCAGCGGCGTGCCCGACAGATTCAGCGGCAGCGGCAGCGGCACCGACTT
CACCCTGAAGATCAGCAGAGTGGAGGCCGAGGACGTGGGCGTGTACTAC
TGCTTCCAGGGCAGCCACGTGCCCCTGACCTTCGGCGCCGGCACCAAGC
TGGAGCTGAAGGGCGCCCCC

In one embodiment, the antigen-binding domain comprises the sequence of SEQ ID NO: 45.

In one embodiment, the antigen-binding domain consists of the sequence of SEQ ID NO: 45.

In one embodiment, the antigen-binding domain comprises a sequence having at least 90% sequence identity to the sequence of SEQ ID NO: 45.

In one embodiment, the antigen-binding domain comprises or consists of a sequence having at least 75% sequence identity to the sequence of SEQ ID NO: 45, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 45.

In one aspect, there is provided a single-chain variable fragment (scFv) comprising:
a) heavy chain variable region (VH) complementarity determining regions (CDRs) with the sequences: CDR1—DYYMY (SEQ ID NO: 42); CDR2—SISFDGTYTYYTDRVKG (SEQ ID NO: 43); and CDR3—DRPAWFPY (SEQ ID NO: 44); or variants thereof each having up to three amino acid substitutions, additions or deletions; and
b) light chain variable region (VL) CDRs with the sequences: CDR1—RSSQSIVHSNGNTYLE (SEQ ID NO: 39); CDR2—KVSNRFS (SEQ ID NO: 40); and CDR3—FQGSHVPLT (SEQ ID NO: 41); or variants thereof each having up to three amino acid substitutions, additions or deletions.

In one embodiment, the scFv comprises the sequence of SEQ ID NO: 45.

In one embodiment, the scFv consists of the sequence of SEQ ID NO: 45.

In one embodiment, the scFv comprises a sequence having at least 90% sequence identity to the sequence of SEQ ID NO: 45.

In one embodiment, the scFv comprises or consists of a sequence having at least 75% sequence identity to the sequence of SEQ ID NO: 45, such as at least 80%, at least 85%, at least 90%, at least 91%, at least 92%, at least 93%, at least 94%, at least 95%, at least 96%, at least 97%, at least 98%, at least 99%, or 100% sequence identity to the sequence of SEQ ID NO: 45.

In one embodiment, the antigen-binding domain comprises:
a) heavy chain variable region (VH) complementarity determining regions (CDRs) with the sequences: CDR1—DHTIHWMR (SEQ ID NO: 1); CDR2—YIYPRDGITGYNERFRGK (SEQ ID NO: 2); and CDR3—WGYSYRNYAYYYDYWGQGTL (SEQ ID NO: 3); or variants thereof each having up to three amino acid substitutions, additions or deletions; and
   light chain variable region (VL) CDRs with the sequences: CDR1—INCRSSQSLLHSSNQR (SEQ ID NO: 4); CDR2—PPKVLIYWASTRES (SEQ ID NO: 5); and CDR3—QQYYSYPWTFGQ (SEQ ID NO: 6); or variants thereof each having up to three amino acid substitutions, additions or deletions; or
b) heavy chain variable region (VH) CDRs with the sequences: CDR1—DYYMY (SEQ ID NO: 42); CDR2—SISFDGTYTYYTDRVKG (SEQ ID NO: 43); and CDR3—DRPAWFPY (SEQ ID NO: 44); or variants thereof each having up to three amino acid substitutions, additions or deletions; and
   light chain variable region (VL) CDRs with the sequences: CDR1—RSSQSIVHSNGNTYLE (SEQ ID NO: 39); CDR2—KVSNRFS (SEQ ID NO: 40); and CDR3—FQGSHVPLT (SEQ ID NO: 41); or variants thereof each having up to three amino acid substitutions, additions or deletions.

### Co-Stimulatory Domain

The CAR of the invention may also comprise one or more co-stimulatory domains. This domain may enhance cell proliferation, cell survival and development of memory cells.

In some embodiments, the co-stimulatory domains are fused together.

In some embodiments, the co-stimulatory domains are separated by linker sequences.

In some embodiments, the co-stimulatory domains are not separated by linker sequences.

Each co-stimulatory domain may comprise the co-stimulatory domain of any one or more of, for example, members of the TNFR super family, CD28, CD137 (4-1BB), CD134 (OX40), DapIO, CD27, CD2, CD5, ICAM-1, LFA-1, Lck, TNFR-1, TNFR-II, Fas, CD30, CD40 or combinations thereof. Co-stimulatory domains from other proteins may also be used with the CAR of the invention. Additional co-stimulatory domains will be apparent to those of skill in the art.

In some embodiments, the co-stimulatory domain is a 4-1BB co-stimulatory domain.

In some embodiments, the co-stimulatory domain is a CD28 co-stimulatory domain.

In some embodiments, the CAR comprises two co-stimulatory domains.

In some embodiments, the one or more co-stimulatory domains comprise a CD28 co-stimulatory domain and a 4-1BB co-stimulatory domain.

In one embodiment, the CD28 co-stimulatory domain and 4-1BB co-stimulatory domains are fused together.

In one embodiment the co-stimulatory domain comprises a sequence having at least 90% sequence identity to the sequence of SEQ ID NO: 60.

Exemplary CD28 co-stimulatory domain
(SEQ ID NO: 60):
RSKRSRLLHSDYMNMTPRRPGPTRKHYQPYAPPRDFAAYRS

In some embodiments, the co-stimulatory domain may comprise a composite co-stimulatory domain comprising one or more co-stimulatory domains according to the invention, or fragments thereof. As such, said domains may be fused in their entirety or smaller sections of said domains may be fused.

In one embodiment, the co-stimulatory domain is a composite co-stimulatory domain comprising a fragment or the entirety of a co-stimulatory domain selected from any two or more of: CD28, CD137 (4-1BB), CD134 (OX40), DapIO, CD27, CD2, CD5, ICAM-1, LFA-1, Lck, TNFR-1, TNFR-II, Fas, CD30, and CD40.

### Intracellular Signaling Domain

The CAR of the invention may also comprise an intracellular signaling domain. This domain may be cytoplasmic and may transduce the effector function signal and direct the cell to perform its specialized function.

Examples of intracellular signaling domains include, but are not limited to, Z chain of the T-cell receptor or any of its homologs (e.g., η chain, FcεR1γ and β chains, MB1 (Igα) chain, B29 (Igβ) chain, etc.), CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may be, for example, human CD3 zeta chain, FcyRIII, FcsRI, cytoplasmic tails of Fc receptors, immunoreceptor tyrosine-based activation motif (ITAM) bearing cytoplasmic receptors or combinations thereof.

Additional intracellular signaling domains will be apparent to those of skill in the art and may be used in connection with alternate embodiments of the invention.

In a preferred embodiment the intracellular signaling domain is a CD3-zeta signalling domain.

In one embodiment the intracellular signaling domain comprises a sequence having at least 90% sequence identity to the sequence of SEQ ID NO: 13.

Exemplary CD3-zeta signalling domain
(SEQ ID NO: 13):
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In one embodiment, the intracellular signaling domain is encoded by SEQ ID NO: 22, or a sequence with at least 75% sequence identity thereto.

Nucleotide sequence encoding exemplary CD3-zeta
signalling domain (SEQ ID NO: 22):
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCTCCTCGCTAA

### Transmembrane Domain

The CAR of the invention may also comprise a transmembrane domain. The transmembrane domain may comprise the transmembrane sequence from any protein which has a transmembrane domain, including any of the type I, type II or type III transmembrane proteins. The transmembrane domain of the CAR of the invention may also comprise an artificial hydrophobic sequence. The transmembrane domains of the CARs of the invention may be selected so as not to dimerize. Additional transmembrane domains will be apparent to those of skill in the art.

Examples of transmembrane (TM) regions used in CAR constructs are: 1) The CD28 TM region (Pule et al, Mol Ther, 2005 November; 12 (5): 933-41; Brentjens et al, CCR, 2007 September 15; 13 (18 Pt 1): 5426-35; Casucci et al, Blood, 2013 November 14; 122 (20): 3461-72.); 2) The OX40 TM region (Pule et al, Mol Ther, 2005 November; 12 (5): 933-41); 3) The 41BB TM region (Brentjens et al, CCR, 2007 September 15; 13 (18 Pt 1): 5426-35); 4) The CD3 zeta TM region (Pule et al, Mol Ther, 2005 November; 12 (5): 933-41; Savoldo B, Blood, 2009 June 18; 113 (25): 6392-402.); 5) The CD8a TM region (Maher et al, Nat Biotechnol, 2002 January; 20 (1): 70-5.; Imai C, Leukemia, 2004 April; 18 (4): 676-84; Brentjens et al, CCR, 2007 September 15; 13 (18 Pt 1): 5426-35; Milone et al, Mol Ther, 2009 August; 17 (8): 1453-64.).

In some embodiments, the transmembrane domain is a CD28 transmembrane domain.

In some embodiments, the transmembrane domain and intracellular co-stimulatory domain may be derived from the same molecule.

In one embodiment the transmembrane domain comprises a sequence with at least 90% sequence identity to the sequence of SEQ ID NO: 61.

Exemplary CD28 transmembrane domain
(SEQ ID NO: 61)
FWVLVVVGGVLACYSLLVTVAFIIFWV

In one embodiment the transmembrane and co-stimulatory domains together comprise a sequence having at least 90% sequence identity to the sequence of SEQ ID NO: 12.

Exemplary CD28 transmembrane and
co-stimulatory domains
(SEQ ID NO: 12)
FWVLVVVGGVLACYSLLVTVAFIIFWVRSKRSRLLHSDYMNMTPR
RPGPTRKHYQPYAPPRDFAAYRS

In some embodiments, the transmembrane domain may comprise a composite transmembrane domain comprising one or more transmembrane domain according to the invention or fragments thereof. As such, said domains may be fused in their entirety or smaller sections of said domains may be fused.

In one embodiment, the transmembrane domain is a composite transmembrane domain comprising a transmembrane domain or fragment thereof from any two or more of the transmembrane domains selected from the group consisting of: CD28 transmembrane domain, CD8a transmembrane domain, 4-1BB transmembrane domain, OX40 transmembrane domain, and CD3 zeta transmembrane domain.

### Spacer Domain

The CAR of the invention may comprise an extracellular spacer domain. The extracellular spacer domain may be attached to the antigen-specific targeting region and the transmembrane domain. The spacer domain may also be referred to as a hinge or linker.

In some embodiments, the spacer is an IgG1-derived hinge spacer.

In one embodiment the spacer comprises a sequence having at least 90% sequence identity to the sequence of SEQ ID NO: 10.

Exemplary IgG1 hinge
(SEQ ID NO: 10)
EPKSCDKTHTCPPCP

In one embodiment, the spacer is encoded by SEQ ID NO: 19, or a sequence with at least 75% sequence identity thereto.

Nucleotide sequence encoding exemplary IgG1
hinge (SEQ ID NO: 19):
GAGCCCAAGAGCTGCGACAAGACCCACACCTGTCCCCCCTGCCCC

In one embodiment the spacer comprises a sequence having at least 90% sequence identity to the sequence of SEQ ID NO: 57.

Exemplary IgG4 hinge
(SEQ ID NO: 57)
ATSSGESKYGPPCPPCP

The CAR of the present invention may comprise an extracellular spacer which comprises at least part of the extracellular domain of human low affinity nerve growth factor receptor (LNGFR) or a derivative thereof.

LNGFR is not expressed on the majority of human hematopoietic cells, thus allowing quantitative analysis of transduced gene expression by immunofluorescence, with single cell resolution. Thus, fluorescence activated cell sorter analysis of expression of LNGFR may be performed in transduced cells to study gene expression. Further details on analysis using LNGFR may be found in Mavilio (1994) Blood 83, 1988-1997.

In one embodiment, the CAR of the invention comprises a truncated LNGFR (also known as ΔLNGFR). Preferably the LNGFR used in the present invention is truncated in its intracytoplasmic domain. Such a truncation is described in Mavilio (1994) Blood 83, 1988-1997.

Thus, preferably the LNGFR spacer of the present invention comprises at least part of the extracellular domain or a derivative thereof but lacks the intracellular domain of LNGFR. The extracellular domain may comprise amino acids 29-250 of LNGFR or a derivative thereof.

Exemplary human LNGFR [UNIPROT accession P08138,
TNR16_HUMAN](SEQ ID NO: 31):
MGAGATGRAMDGPRLLLLLLLGVSLGGAKEACPTGLYTHSGECCK
ACNLGEGVAQPCGANQTVCEPCLDSVTFSDVVSATEPCKPCTECV
GLQSMSAPCVEADDAVCRCAYGYYQDETTGRCEACRVCEAGSGLV
FSCQDKQNTVCEECPDGTYSDEANHVDPCLPCTVCEDTERQLREC
TRWADAECEEIPGRWITRSTPPEGSDSTAPSTQEPEAPPEQDLIA
STVAGVVTTVMGSSQPVVTRGTTDNLIPVYCSILAAVVVGLVAYI
AFKRWNSCKQNKQGANSRPVNQTPPPEGEKLHSDSGISVDSQSLH
DQQPHTQTASGQALKGDGGLYSSLPPAKREEVEKLLNGSAGDTWR
HLAGELGYQPEHIDSFTHEACPVRALLASWATQDSATLDALLAAL
RRIQRADLVESLCSESTATSPV
Exemplary extracellular domain of the human
LNGFR [UNIPROT accession P08138,
TNR16_HUMAN, position 29-250]
(SEQ ID NO: 27)
KEACPTGLYTHSGECCKACNLGEGVAQPCGANQTVCEPCLDSVTF
SDVVSATEPCKPCTECVGLQSMSAPCVEADDAVCRCAYGYYQDET
TGRCEACRVCEAGSGLVFSCQDKQNTVCEECPDGTYSDEANHVDP
CLPCTVCEDTERQLRECTRWADAECEEIPGRWITRSTPPEGSDST
APSTQEPEAPPEQDLIASTVAGVVTTVMGSSQPVVTRGTTDN

Preferably the LNGFR lacks the signal peptide.

SEQ ID NO: 27 may also be referred to as the LNGFR wild type long spacer (NWL).

In one embodiment, the spacer comprises at least part of a protein having at least 75%, 80%, 85%, 90%, 95%, 96%, 97%, 98%, 99% or 100% identity to the extracellular domain of LNGFR (e.g., SEQ ID NO: 27). In one embodiment, the spacer comprises at least part of a protein having at least 75%, 80%, 85%, 90%, 95%, 96%, 97%, 98%, 99% or 100% identity to amino acids 29-250 of the LNGFR protein (e.g., SEQ ID NO: 31).

In one embodiment, the spacer comprises a protein having at least 75%, 80%, 85%, 90%, 95%, 96%, 97%, 98%, 99% or 100% identity to SEQ ID NO: 27. In one embodiment, the spacer comprises a protein having at least 75%, 80%, 85%, 90%, 95%, 96%, 97%, 98%, 99% or 100% identity to amino acids 29-250 of SEQ ID NO: 31.

In one embodiment, the spacer is encoded by SEQ ID NO: 29, or a sequence with at least 75% sequence identity thereto.

Nucleotide sequence encoding exemplary LNGFR
spacer (NWL) (SEQ ID NO: 29):
AAAGAGGCCTGCCCCACCGGCCTGTACACCCACAGCGGAGAGTGC
TGCAAGGCCTGCAACCTGGGAGAGGGCGTGGCCCAGCCTTGCGGC
GCCAATCAGACCGTGTGCGAGCCCTGCCTGGACAGCGTGACCTTC
AGCGACGTGGTGTCCGCCACCGAGCCCTGCAAGCCTTGCACCGAG
TGTGTGGGCCTGCAGAGCATGAGCGCCCCCTGCGTGGAAGCCGAC
GACGCCGTGTGTAGATGCGCCTACGGCTACTACCAGGACGAGACA
ACCGGCAGATGCGAGGCCTGTAGAGTGTGCGAGGCCGGCAGCGGC
CTGGTGTTCAGTTGTCAAGACAAGCAGAATACCGTGTGTGAAGAG
TGCCCCGACGGCACCTACAGCGACGAGGCCAACCACGTGGACCCC
TGCCTGCCCTGCACTGTGTGCGAGGACACCGAGCGGCAGCTGCGC
GAGTGCACAAGATGGGCCGACGCCGAGTGCGAAGAGATCCCCGGC
AGATGGATCACCAGAAGCACCCCCCCTGAGGGCAGCGACAGCACC
GCCCCTAGCACCCAGGAACCTGAGGCCCCTCCCGAGCAGGACCTG
ATCGCCTCTACAGTGGCCGGCGTGGTGACAACCGTGATGGGCAGC
TCTCAGCCCGTGGTGACACGGGGCACCACCGACAAT

In one embodiment, the spacer comprises a protein having at least 75%, 80%, 85%, 90%, 95%, 96%, 97%, 98%, 99% or 100% identity to SEQ ID NO: 11.

Exemplary LNGFR spacer
(SEQ ID NO: 11)
KEACPTGLYTHSGECCKACNLGEGVAQPCGANQTVCEPCLDSVTF
SDVVSATEPCKPCTECVGLQSMSAPCVEADDAVCRCAYGYYQDET
TGRCEACRVCEAGSGLVFSCQDKQNTVCEECPDGTYSDEAARAAD
AECEE

SEQ ID NO: 11 may also be referred to as the LNGFR mutated short spacer (NMS)

In one embodiment, the spacer is encoded by SEQ ID NO: 20, or a sequence with at least 75% sequence identity thereto.

Nucleotide sequence encoding exemplary
LNGFR spacer (SEQ ID NO: 20):
AAAGAGGCCTGCCCCACCGGCCTGTACACCCACAGCGGAGAGTGC
TGCAAGGCCTGCAACCTGGGAGAGGGCGTGGCCCAGCCTTGCGGC
GCCAATCAGACCGTGTGCGAGCCCTGCCTGGACAGCGTGACCTTC
AGCGACGTGGTGTCCGCCACCGAGCCCTGCAAGCCTTGCACCGAG
TGTGTGGGCCTGCAGAGCATGAGCGCCCCCTGCGTGGAAGCCGAC
GACGCCGTGTGTAGATGCGCCTACGGCTACTACCAGGACGAGACA
ACCGGCAGATGCGAGGCCTGTAGAGTGTGCGAGGCCGGCAGCGGC
CTGGTGTTCAGTTGTCAGGACAAGCAGAACACCGTGTGTGAAGAG
TGCCCCGACGGCACCTACAGCGACGAGGCCGCCCGGGCCGCCGAC
GCCGAGTGCGAGGAA

Further exemplary spacers are illustrated below.

Exemplary LNGFR spacer (LNGFR wild type
short (NWS))
(SEQ ID NO: 32)
KEACPTGLYTHSGECCKACNLGEGVAQPCGANQTVCEPCLDSVTF
SDVVSATEPCKPCTECVGLQSMSAPCVEADDAVCRCAYGYYQDET
TGRCEACRVCEAGSGLVFSCQDKQNTVCEECPDGTYSDEANHVDP
CLPCTVCEDTERQLRECTRWADAECEE
Exemplary LNGFR spacer (LNGFR mutated
long (NML))
(SEQ ID NO: 33)
KEACPTGLYTHSGECCKACNLGEGVAQPCGANQTVCEPCLDSVTF
SDVVSATEPCKPCTECVGLQSMSAPCVEADDAVCRCAYGYYQDET
TGRCEACRVCEAGSGLVFSCQDKQNTVCEECPDGTYSDEAARAAD
AECEEIPGRWITRSTPPEGSDSTAPSTQEPEAPPEQDLIASTVAG
VVTTVMGSSQPVVTRGTTDN

In one embodiment the spacer comprises a sequence having at least 75%, 80%, 85%, 90%, 95%, 96%, 97%, 98%, 99% or 100% identity to any one of SEQ ID NOs: 11, 31, or 32.

LNGFR comprises 4 TNFR-Cys domains (TNFR-Cys 1, TNFR-Cys 2, TNFR-Cys 3 and TNFR-Cys 4). Sequences of the domains are exemplified below:

TNFR-Cys 1
(SEQ ID NO: 34)
ACPTGLYTHSGECCKACNLGEGVAQPCGANQTVC
TNFR-Cys 2
(SEQ ID NO: 35)
PCLDSVTFSDVVSATEPCKPCTECVGLQSMSAPCVEADDAVC
TNFR-Cys 3
(SEQ ID NO: 36)
RCAYGYYQDETTGRCEACRVCEAGSGLVFSCQDKQNTVC
TNFR-Cys 4
(SEQ ID NO: 37)
ECPDGTYSDEANHVDPCLPCTVCEDTERQLRECTRWADAEC

In one embodiment, the spacer comprises TNFR-Cys 1, 2 and 3 domains or fragments or derivatives thereof. In another embodiment, the spacer comprises the TNFR-Cys 1, 2, 3 and 4 domains or fragments or derivatives thereof.

In one embodiment the spacer comprises a sequence having at least 80%, 85%, 90%, 95%, 96%, 97%, 98%, 99% identity or 100% identity to TNFR-Cys 1 (SEQ ID NO: 34), a sequence having at least 80%, 85%, 90%, 95%, 96%, 97%, 98%, 99% identity or 100% identity to TNFR-Cys 2 (SEQ ID NO: 35), or a sequence having at least 80%, 85%, 90%, 95%, 96%, 97%, 98%, 99% identity or 100% identity to TNFR-Cys 3 (SEQ ID NO: 36). The spacer may further comprise a sequence having at least 80%, 85%, 90%, 95%, 96%, 97%, 98%, 99% identity or 100% identity to TNFR-Cys 4 (SEQ ID NO: 37).

Rather than comprise the full TNFR-Cys 4 domain, the spacer may comprise a TNFR-Cys 4 domain with the following amino acids deleted from said domain:

(SEQ ID NO: 38)
NHVDPCLPCTVCEDTERQLRECTRW

In one embodiment, the NHVDPCLPCTVCEDTERQLRECTRW (SEQ ID NO: 38) amino acids are replaced with the following amino acids:
ARA.

In one embodiment the spacer lacks the LNGFR serine/threonine-rich stalk. In another embodiment the spacer comprises the LNGFR serine/threonine-rich stalk.

The spacer may comprise or consist of a sequence of SEQ ID NO: 34 or a sequence having at least 85%, 90%, 95%, 96%, 97%, 98% or 99% identity to SEQ ID NO: 34.

The spacer may comprise or consist of a sequence of SEQ ID NO: 35 or a sequence having at least 85%, 90%, 95%, 96%, 97%, 98% or 99% identity to SEQ ID NO: 35.

The spacer may comprise or consist of a sequence of SEQ ID NO: 36 or a sequence having at least 85%, 90%, 95%, 96%, 97%, 98% or 99% identity to SEQ ID NO: 36.

The spacer may comprise or consist of a sequence of SEQ ID NO: 37 or a sequence having at least 85%, 90%, 95%, 96%, 97%, 98% or 99% identity to SEQ ID NO: 37.

The spacer may comprise a mutated version of the IgG1 CH2CH3 spacer (mCH2CH₃) that is unable to recognize the FcγRI (Hombach et al., Gene Ther. 2000).

In one embodiment, the spacer comprises a protein having at least 75%, 80%, 85%, 90%, 95%, 96%, 97%, 98%, 99% or 100% identity to SEQ ID NO: 28.

Exemplary mCH2CH3 spacer (SEQ ID NO: 28):
EPKSPDKTHTCPPCPAPPVAGPSVFLFPPKPKDTLMIARTPEVTC
VVVDVSHEDPEVKFNWYVDGVEVHNAKTKPREEQYNSTYRVVSVL
TVLHQDWLNGKEYKCKVSNKALPAPIEKTISKAKGQPREPQVYTL
PPSRDELTKNQVSLTCLVKGFYPSDIAVEWESNGQPENNYKTTPP
VLDSDGSFFLYSKLTVDKSRWQQGNVFSCSVMHEALHNHYTQKSL
SLSPGK

In one embodiment, the spacer is encoded by SEQ ID NO: 30, or a sequence with at least 75% sequence identity thereto.

Nucleotide sequence encoding exemplary mCH2CH3
spacer (SEQ ID NO: 30):
GAGCCCAAGAGCCCCGACAAGACCCACACCTGTCCCCCCTGTCCT
GCCCCTCCAGTGGCCGGACCTAGCGTGTTCCTGTTCCCCCCAAAG
CCCAAGGACACCCTGATGATCGCCCGGACCCCCGAAGTGACCTGC
GTGGTGGTGGACGTGTCCCACGAGGACCCTGAAGTGAAGTTCAAT
TGGTACGTGGACGGCGTGGAAGTGCACAACGCCAAGACCAAGCCC
AGAGAGGAACAGTACAACAGCACCTACCGGGTGGTGTCCGTGCTG
ACCGTGCTGCACCAGGACTGGCTGAACGGCAAAGAATACAAGTGC
AAGGTCTCCAACAAGGCCCTGCCTGCCCCCATCGAGAAAACCATC
AGCAAGGCCAAGGGCCAGCCCCGCGAGCCCCAGGTGTACACACTG
CCCCCCAGCCGGGACGAGCTGACCAAGAACCAGGTGTCCCTGACC
TGCCTCGTGAAAGGCTTCTACCCCAGCGATATCGCCGTGGAATGG
GAGAGCAACGGCCAGCCCGAGAACAACTACAAGACCACCCCCCCT
GTGCTGGACAGCGACGGCTCATTCTTCCTGTACAGCAAGCTGACC
GTGGACAAGAGCCGGTGGCAGCAGGGCAACGTGTTCAGCTGCAGC
GTGATGCACGAGGCCCTGCACAACCACTACACCCAGAAGTCCCTG
AGCCTGAGCCCCGGCAAG

The spacer may confer properties to the CAR such that it allows for immunoselection of cells, preferably T-cells, expressing said CAR.

The CAR of the present invention (e.g. comprising the spacer referred to herein) preferably enables T-cells expressing the CAR to proliferate in the presence of cells expressing the antigen for which the CAR is designed.

The CAR of the present invention (e.g. comprising the spacer referred to herein) preferably enables T-cells expressing the CAR to mediate therapeutically significant anti-cancer effects against a cancer that the CAR is designed to target.

The CAR of the present invention (e.g. comprising the spacer referred to herein) is preferably suitable for facilitating immunoselection of cells transduced with said CAR.

An exemplary CAR of the present invention comprising the LNGFR-based spacer may avoid activation of unwanted and potentially toxic off-target immune responses and may allow CAR-expressing T cells to persist in vivo without being prematurely cleared by the host immune system.

As described herein, the present invention also encompasses the use of variants, derivatives, homologues and fragments of the spacer elements described herein.

### Exemplary CAR

In one aspect, the CAR is an anti-CDH17 CAR

In one embodiment, the CAR comprises:
a) a CD28, a CD8, or a CD4 transmembrane domain;
b) an IgG1 hinge, LNGFR spacer or mCH2CH3 spacer;
c) a CD28 and/or a 4-1BB co-stimulatory domain;
d) a CD3-zeta signalling domain; and/or
e) an anti-CDH17 scFv.

In one embodiment, the CAR comprises an antigen-binding domain comprising an anti-CDH17 scFv, IgG1 hinge, CD28 transmembrane and co-stimulatory domain and a CD3-zeta signalling domain.

In one embodiment, the CAR comprises a protein having at least 75%, 80%, 85%, 90%, 95%, 96%, 97%, 98%, 99% or 100% sequence identity to SEQ ID NO: 14.

Exemplary CAR [A4_4R-hinge.28z] (SEQ ID NO: 14):
MEAPAQLLFLLLLWLPDTTGDIVMTQSPDSLAVSLGERATINCRSSQSL
LHSSNQRNYLAWYQQKPGQPPKVLIYWASTRESGVPDRFSGSGSGTDFT
LTISSLQAEDVAVYYCQQYYSYPWTFGQGTKVEIKGGGGSGGGGSGGGG
SQVQLVQSGAEVKKPGASVKVSCKASGYTLTDHTIHWMRQAPGQRLEWI
GYIYPRDGITGYNERFRGKATLTADTSASTAYMELSSLRSEDTAVYYCA
RWGYSYRNYAYYYDYWGQGTLVTVSSSPVEPKSCDKTHTCPPCPPLIKF
WVLVVVGGVLACYSLLVTVAFIIFWVRSKRSRLLHSDYMNMTPRRPGPT
RKHYQPYAPPRDFAAYRSRVKFSRSADAPAYQQGQNQLYNELNLGRREE
YDVLDKRRGRDPEMGGKPRRKNPQEGLYNELQKDKMAEAYSEIGMKGER
RRGKGHDGLYQGLSTATKDTYDALHMQALPPR

In one embodiment, the CAR comprises a protein that is encoded by a nucleotide sequence having at least 75%, 80%, 85%, 90%, 95%, 96%, 97%, 98%, 99% or 100% sequence identity to SEQ ID NO: 17.

Nucleotide sequence encoding exemplary CAR [A4_
4R-hinge.28z] (SEQ ID NO: 17):
ATGGAGGCCCCCGCCCAGCTGCTGTTCCTGCTGCTGCTGTGGCTGCCCG
ACACCACCGGCGACATCGTGATGACCCAGAGCCCCGACAGCCTGGCCGT
GAGCCTGGGCGAGAGAGCCACCATCAACTGCAGAAGCAGCCAGAGCCTG
CTGCACAGCAGCAACCAGAGAAACTACCTGGCCTGGTACCAGCAGAAGC
CCGGCCAGCCCCCCAAGGTGCTGATCTACTGGGCCAGCACCAGAGAGAG
CGGCGTGCCCGACAGATTCAGCGGCAGCGGCAGCGGCACCGACTTCACC
CTGACCATCAGCAGCCTGCAGGCCGAGGACGTGGCCGTGTACTACTGCC
AGCAGTACTACAGCTACCCCTGGACCTTCGGCCAGGGCACCAAGGTGGA
GATCAAGGGCGGCGGCGGCAGCGGCGGCGGCGGCAGCGGCGGCGGCGGC
AGCCAGGTGCAGCTGGTGCAGAGCGGCGCCGAGGTGAAGAAGCCCGGCG
CCAGCGTGAAGGTGAGCTGCAAGGCCAGCGGCTACACCCTGACCGACCA
CACCATCCACTGGATGAGACAGGCCCCCGGCCAGAGACTGGAGTGGATC
GGCTACATCTACCCCAGAGACGGCATCACCGGCTACAACGAGAGATTCA
GAGGCAAGGCCACCCTGACCGCCGACACCAGCGCCAGCACCGCCTACAT
GGAGCTGAGCAGCCTGAGAAGCGAGGACACCGCCGTGTACTACTGCGCC
AGATGGGGCTACAGCTACAGAAACTACGCCTACTACTACGACTACTGGG
GCCAGGGCACCCTGGTGACCGTGAGCAGCTCAccggTCGAGCCCAAGAG
CTGCGACAAGACCCACACCTGTCCCCCCTGCCCCCCCTTAATTAAAttT
TGGGTGCTGGTGGTGGTTGGTGGAGTCCTGGCTTGCTATAGCTTGCTAG
TAACAGTGGCCTTTATTATTTTCTGGGTGAGGAGTAAGAGGAGCAGGCT
CCTGCACAGTGACTACATGAACATGACTCCCCGCCGCCCCGGGCCCACC
CGCAAGCATTACCAGCCCTATGCCCCACCACGCGACTTCGCAGCCTATC
GCTCCAGAGTGAAGTTCAGCAGGAGCGCAGACGCCCCCGCGTACCAGCA
GGGCCAGAACCAGCTCTATAACGAGCTCAATCTAGGACGAAGAGAGGAG
TACGATGTTTTGGACAAGAGACGTGGCCGGGACCCTGAGATGGGGGGAA
AGCCGAGAAGGAAGAACCCTCAGGAAGGCCTGTACAATGAACTGCAGAA
AGATAAGATGGCGGAGGCCTACAGTGAGATTGGGATGAAAGGCGAGCGC
CGGAGGGGCAAGGGGCACGATGGCCTTTACCAGGGTCTCAGTACAGCCA
CCAAGGACACCTACGACGCCCTTCACATGCAGGCCCTGCCTCCTCGCTA
A

In one embodiment, the CAR comprises an antigen-binding domain comprising an anti-CDH17 scFv, LNGFR mutated short spacer (NMS), CD28 transmembrane and co-stimulatory domain and a CD3-zeta signalling domain.

In one embodiment, the CAR comprises a protein having at least 75%, 80%, 85%, 90%, 95%, 96%, 97%, 98%, 99% or 100% sequence identity to SEQ ID NO: 15.

Exemplary CAR [A4_4R-NMS.28z] (SEQ ID NO: 15):
MEAPAQLLFLLLLWLPDTTGDIVMTQSPDSLAVSLGERATINCRSSQSL
LHSSNQRNYLAWYQQKPGQPPKVLIYWASTRESGVPDRFSGSGSGTDFT
LTISSLQAEDVAVYYCQQYYSYPWTFGQGTKVEIKGGGGSGGGGSGGGG
SQVQLVQSGAEVKKPGASVKVSCKASGYTLTDHTIHWMRQAPGQRLEWI
GYIYPRDGITGYNERFRGKATLTADTSASTAYMELSSLRSEDTAVYYCA
RWGYSYRNYAYYYDYWGQGTLVTVSSSPVKEACPTGLYTHSGECCKACN
LGEGVAQPCGANQTVCEPCLDSVTFSDVVSATEPCKPCTECVGLQSMSA
PCVEADDAVCRCAYGYYQDETTGRCEACRVCEAGSGLVFSCQDKQNTVC
EECPDGTYSDEAARAADAECEEPLIKFWVLVVVGGVLACYSLLVTVAFI
IFWVRSKRSRLLHSDYMNMTPRRPGPTRKHYQPYAPPRDFAAYRSRVKF
SRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYD
ALHMQALPPR

In one embodiment, the CAR comprises a protein that is encoded by a nucleotide sequence having at least 75%, 80%, 85%, 90%, 95%, 96%, 97%, 98%, 99% or 100% sequence identity to SEQ ID NO: 18.

Nucleotide sequence encoding exemplary CAR [A4_4R-NMS.28z] (SEQ
ID NO: 18):
ATGGAGGCCCCCGCCCAGCTGCTGTTCCTGCTGCTGCTGTGGCTGCCCGACAC
CACCGGCGACATCGTGATGACCCAGAGCCCCGACAGCCTGGCCGTGAGCCTG
GGCGAGAGAGCCACCATCAACTGCAGAAGCAGCCAGAGCCTGCTGCACAGCA
GCAACCAGAGAAACTACCTGGCCTGGTACCAGCAGAAGCCCGGCCAGCCCCC
CAAGGTGCTGATCTACTGGGCCAGCACCAGAGAGAGCGGCGTGCCCGACAGA
TTCAGCGGCAGCGGCAGCGGCACCGACTTCACCCTGACCATCAGCAGCCTGCA
GGCCGAGGACGTGGCCGTGTACTACTGCCAGCAGTACTACAGCTACCCCTGGA
CCTTCGGCCAGGGCACCAAGGTGGAGATCAAGGGCGGCGGCGGCAGCGGCG
GCGGCGGCAGCGGCGGCGGCGGCAGCCAGGTGCAGCTGGTGCAGAGCGGCG
CCGAGGTGAAGAAGCCCGGCGCCAGCGTGAAGGTGAGCTGCAAGGCCAGCGG
CTACACCCTGACCGACCACACCATCCACTGGATGAGACAGGCCCCCGGCCAGA
GACTGGAGTGGATCGGCTACATCTACCCCAGAGACGGCATCACCGGCTACAAC
GAGAGATTCAGAGGCAAGGCCACCCTGACCGCCGACACCAGCGCCAGCACCG
CCTACATGGAGCTGAGCAGCCTGAGAAGCGAGGACACCGCCGTGTACTACTGC
GCCAGATGGGGCTACAGCTACAGAAACTACGCCTACTACTACGACTACTGGGG
CCAGGGCACCCTGGTGACCGTGAGCAGCTCAccggTCAAAGAGGCCTGCCCCAC
CGGCCTGTACACCCACAGCGGAGAGTGCTGCAAGGCCTGCAACCTGGGAGAG
GGCGTGGCCCAGCCTTGCGGCGCCAATCAGACCGTGTGCGAGCCCTGCCTGG
ACAGCGTGACCTTCAGCGACGTGGTGTCCGCCACCGAGCCCTGCAAGCCTTGC
ACCGAGTGTGTGGGCCTGCAGAGCATGAGCGCCCCCTGCGTGGAAGCCGACG
ACGCCGTGTGTAGATGCGCCTACGGCTACTACCAGGACGAGACAACCGGCAGA
TGCGAGGCCTGTAGAGTGTGCGAGGCCGGCAGCGGCCTGGTGTTCAGTTGTC
AGGACAAGCAGAACACCGTGTGTGAAGAGTGCCCCGACGGCACCTACAGCGAC
GAGGCCGCCCGGGCCGCCGACGCCGAGTGCGAGGAACCCTTAATTAAAttTTGG
GTGCTGGTGGTGGTTGGTGGAGTCCTGGCTTGCTATAGCTTGCTAGTAACAGT
GGCCTTTATTATTTTCTGGGTGAGGAGTAAGAGGAGCAGGCTCCTGCACAGTGA
CTACATGAACATGACTCCCCGCCGCCCCGGGCCCACCCGCAAGCATTACCAGC
CCTATGCCCCACCACGCGACTTCGCAGCCTATCGCTCCAGAGTGAAGTTCAGC
AGGAGCGCAGACGCCCCCGCGTACCAGCAGGGCCAGAACCAGCTCTATAACG
AGCTCAATCTAGGACGAAGAGAGGAGTACGATGTTTTGGACAAGAGACGTGGC
CGGGACCCTGAGATGGGGGGAAAGCCGAGAAGGAAGAACCCTCAGGAAGGCC
TGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTC
TCAGTACAGCCACCAAGGACACCTACGACGCCCTTCACATGCAGGCCCTGCCT
CCTCGCTAA

In one embodiment, the CAR comprises an antigen-binding domain comprising an anti-CDH17 scFv, LNGFR wildtype long spacer (NWL), CD28 transmembrane and co-stimulatory domain and a CD3-zeta signalling domain.

In one embodiment, the CAR comprises a protein having at least 75%, 80%, 85%, 90%, 95%, 96%, 97%, 98%, 99% or 100% sequence identity to SEQ ID NO: 23.

Exemplary CAR [A4_4R-NWL.28z] (SEQ ID NO: 23):
MEAPAQLLFLLLLWLPDTTGDIVMTQSPDSLAVSLGERATINCRSSQSL
LHSSNQRNYLAWYQQKPGQPPKVLIYWASTRESGVPDRFSGSGSGTDFT
LTISSLQAEDVAVYYCQQYYSYPWTFGQGTKVEIKGGGGSGGGGSGGGG
SQVQLVQSGAEVKKPGASVKVSCKASGYTLTDHTIHWMRQAPGQRLEWI
GYIYPRDGITGYNERFRGKATLTADTSASTAYMELSSLRSEDTAVYYCA
RWGYSYRNYAYYYDYWGQGTLVTVSSSPVKEACPTGLYTHSGECCKACN
LGEGVAQPCGANQTVCEPCLDSVTFSDVVSATEPCKPCTECVGLQSMSA
PCVEADDAVCRCAYGYYQDETTGRCEACRVCEAGSGLVFSCQDKQNTVC
EECPDGTYSDEANHVDPCLPCTVCEDTERQLRECTRWADAECEEIPGRW
ITRSTPPEGSDSTAPSTQEPEAPPEQDLIASTVAGVVTTVMGSSQPVVT
RGTTDNPLIKFWVLVVVGGVLACYSLLVTVAFIIFWVRSKRSRLLHSDY
MNMTPRRPGPTRKHYQPYAPPRDFAAYRSRVKFSRSADAPAYQQGQNQL
YNELNLGRREEYDVLDKRRGRDPEMGGKPRRKNPQEGLYNELQKDKMAE
AYSEIGMKGERRRGKGHDGLYQGLSTATKDTYDALHMQALPPR

In one embodiment, the CAR comprises a protein that is encoded by a nucleotide sequence having at least 75%, 80%, 85%, 90%, 95%, 96%, 97%, 98%, 99% or 100% sequence identity to SEQ ID NO: 25.

Nucleotide sequence encoding exemplary CAR [A4_4R-NWL.28z] (SEQ
ID NO: 25):
ATGGAGGCCCCCGCCCAGCTGCTGTTCCTGCTGCTGCTGTGGCTGCCCGACAC
CACCGGCGACATCGTGATGACCCAGAGCCCCGACAGCCTGGCCGTGAGCCTG
GGCGAGAGAGCCACCATCAACTGCAGAAGCAGCCAGAGCCTGCTGCACAGCA
GCAACCAGAGAAACTACCTGGCCTGGTACCAGCAGAAGCCCGGCCAGCCCCC
CAAGGTGCTGATCTACTGGGCCAGCACCAGAGAGAGCGGCGTGCCCGACAGA
TTCAGCGGCAGCGGCAGCGGCACCGACTTCACCCTGACCATCAGCAGCCTGCA
GGCCGAGGACGTGGCCGTGTACTACTGCCAGCAGTACTACAGCTACCCCTGGA
CCTTCGGCCAGGGCACCAAGGTGGAGATCAAGGGCGGCGGCGGCAGCGGCG
GCGGCGGCAGCGGCGGCGGCGGCAGCCAGGTGCAGCTGGTGCAGAGCGGCG
CCGAGGTGAAGAAGCCCGGCGCCAGCGTGAAGGTGAGCTGCAAGGCCAGCGG
CTACACCCTGACCGACCACACCATCCACTGGATGAGACAGGCCCCCGGCCAGA
GACTGGAGTGGATCGGCTACATCTACCCCAGAGACGGCATCACCGGCTACAAC
GAGAGATTCAGAGGCAAGGCCACCCTGACCGCCGACACCAGCGCCAGCACCG
CCTACATGGAGCTGAGCAGCCTGAGAAGCGAGGACACCGCCGTGTACTACTGC
GCCAGATGGGGCTACAGCTACAGAAACTACGCCTACTACTACGACTACTGGGG
CCAGGGCACCCTGGTGACCGTGAGCAGCTCAccggTCAAAGAGGCCTGCCCCAC
CGGCCTGTACACCCACAGCGGAGAGTGCTGCAAGGCCTGCAACCTGGGAGAG
GGCGTGGCCCAGCCTTGCGGCGCCAATCAGACCGTGTGCGAGCCCTGCCTGG
ACAGCGTGACCTTCAGCGACGTGGTGTCCGCCACCGAGCCCTGCAAGCCTTGC
ACCGAGTGTGTGGGCCTGCAGAGCATGAGCGCCCCCTGCGTGGAAGCCGACG
ACGCCGTGTGTAGATGCGCCTACGGCTACTACCAGGACGAGACAACCGGCAGA
TGCGAGGCCTGTAGAGTGTGCGAGGCCGGCAGCGGCCTGGTGTTCAGTTGTC
AAGACAAGCAGAATACCGTGTGTGAAGAGTGCCCCGACGGCACCTACAGCGAC
GAGGCCAACCACGTGGACCCCTGCCTGCCCTGCACTGTGTGCGAGGACACCG
AGCGGCAGCTGCGCGAGTGCACAAGATGGGCCGACGCCGAGTGCGAAGAGAT
CCCCGGCAGATGGATCACCAGAAGCACCCCCCCTGAGGGCAGCGACAGCACC
GCCCCTAGCACCCAGGAACCTGAGGCCCCTCCCGAGCAGGACCTGATCGCCT
CTACAGTGGCCGGCGTGGTGACAACCGTGATGGGCAGCTCTCAGCCCGTGGT
GACACGGGGCACCACCGACAATCCCTTAATTAAAttTTGGGTGCTGGTGGTGGTT
GGTGGAGTCCTGGCTTGCTATAGCTTGCTAGTAACAGTGGCCTTTATTATTTTCT
GGGTGAGGAGTAAGAGGAGCAGGCTCCTGCACAGTGACTACATGAACATGACT
CCCCGCCGCCCCGGGCCCACCCGCAAGCATTACCAGCCCTATGCCCCACCAC
GCGACTTCGCAGCCTATCGCTCCAGAGTGAAGTTCAGCAGGAGCGCAGACGCC
CCCGCGTACCAGCAGGGCCAGAACCAGCTCTATAACGAGCTCAATCTAGGACG
AAGAGAGGAGTACGATGTTTTGGACAAGAGACGTGGCCGGGACCCTGAGATGG
GGGGAAAGCCGAGAAGGAAGAACCCTCAGGAAGGCCTGTACAATGAACTGCAG
AAAGATAAGATGGCGGAGGCCTACAGTGAGATTGGGATGAAAGGCGAGCGCCG
GAGGGGCAAGGGGCACGATGGCCTTTACCAGGGTCTCAGTACAGCCACCAAG
GACACCTACGACGCCCTTCACATGCAGGCCCTGCCTCCTCGCTAA

In one embodiment, the CAR comprises an antigen-binding domain comprising an anti-CDH17 scFv, mCH2CH3 spacer, CD28 transmembrane and co-stimulatory domain and a CD3-zeta signalling domain.

In one embodiment, the CAR comprises a protein having at least 75%, 80%, 85%, 90%, 95%, 96%, 97%, 98%, 99% or 100% sequence identity to SEQ ID NO: 24.

Exemplary CAR [A4_4R-CH2CH3mut.28z] (SEQ ID NO:
24):
MEAPAQLLFLLLLWLPDTTGDIVMTQSPDSLAVSLGERATINCRSSQSL
LHSSNQRNYLAWYQQKPGQPPKVLIYWASTRESGVPDRFSGSGSGTDFT
LTISSLQAEDVAVYYCQQYYSYPWTFGQGTKVEIKGGGGSGGGGSGGGG
SQVQLVQSGAEVKKPGASVKVSCKASGYTLTDHTIHWMRQAPGQRLEWI
GYIYPRDGITGYNERFRGKATLTADTSASTAYMELSSLRSEDTAVYYCA
RWGYSYRNYAYYYDYWGQGTLVTVSSSPVEPKSPDKTHTCPPCPAPPVA
GPSVFLFPPKPKDTLMIARTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGKPLIKFWVLVVVGGVLACYSLLVTVAFIIFWVRSK
RSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQAL
PPR

In one embodiment, the CAR comprises a protein that is encoded by a nucleotide sequence having at least 75%, 80%, 85%, 90%, 95%, 96%, 97%, 98%, 99% or 100% sequence identity to SEQ ID NO: 26.

Nucleotide sequence encoding exemplary CAR [A4_4R-CH2CH3mut.28z]
(SEQ ID NO: 26):
ATGGAGGCCCCCGCCCAGCTGCTGTTCCTGCTGCTGCTGTGGCTGCCCGACAC
CACCGGCGACATCGTGATGACCCAGAGCCCCGACAGCCTGGCCGTGAGCCTG
GGCGAGAGAGCCACCATCAACTGCAGAAGCAGCCAGAGCCTGCTGCACAGCA
GCAACCAGAGAAACTACCTGGCCTGGTACCAGCAGAAGCCCGGCCAGCCCCC
CAAGGTGCTGATCTACTGGGCCAGCACCAGAGAGAGCGGCGTGCCCGACAGA
TTCAGCGGCAGCGGCAGCGGCACCGACTTCACCCTGACCATCAGCAGCCTGCA
GGCCGAGGACGTGGCCGTGTACTACTGCCAGCAGTACTACAGCTACCCCTGGA
CCTTCGGCCAGGGCACCAAGGTGGAGATCAAGGGCGGCGGCGGCAGCGGCG
GCGGCGGCAGCGGCGGCGGCGGCAGCCAGGTGCAGCTGGTGCAGAGCGGCG
CCGAGGTGAAGAAGCCCGGCGCCAGCGTGAAGGTGAGCTGCAAGGCCAGCGG
CTACACCCTGACCGACCACACCATCCACTGGATGAGACAGGCCCCCGGCCAGA
GACTGGAGTGGATCGGCTACATCTACCCCAGAGACGGCATCACCGGCTACAAC
GAGAGATTCAGAGGCAAGGCCACCCTGACCGCCGACACCAGCGCCAGCACCG
CCTACATGGAGCTGAGCAGCCTGAGAAGCGAGGACACCGCCGTGTACTACTGC
GCCAGATGGGGCTACAGCTACAGAAACTACGCCTACTACTACGACTACTGGGG
CCAGGGCACCCTGGTGACCGTGAGCAGCTCaccggTCGAGCCCAAGAGCCCCG
ACAAGACCCACACCTGTCCCCCCTGTCCTGCCCCTCCAGTGGCCGGACCTAGC
GTGTTCCTGTTCCCCCCAAAGCCCAAGGACACCCTGATGATCGCCCGGACCCC
CGAAGTGACCTGCGTGGTGGTGGACGTGTCCCACGAGGACCCTGAAGTGAAGT
TCAATTGGTACGTGGACGGCGTGGAAGTGCACAACGCCAAGACCAAGCCCAGA
GAGGAACAGTACAACAGCACCTACCGGGTGGTGTCCGTGCTGACCGTGCTGCA
CCAGGACTGGCTGAACGGCAAAGAATACAAGTGCAAGGTCTCCAACAAGGCCC
TGCCTGCCCCCATCGAGAAAACCATCAGCAAGGCCAAGGGCCAGCCCCGCGA
GCCCCAGGTGTACACACTGCCCCCCAGCCGGGACGAGCTGACCAAGAACCAG
GTGTCCCTGACCTGCCTCGTGAAAGGCTTCTACCCCAGCGATATCGCCGTGGA
ATGGGAGAGCAACGGCCAGCCCGAGAACAACTACAAGACCACCCCCCCTGTGC
TGGACAGCGACGGCTCATTCTTCCTGTACAGCAAGCTGACCGTGGACAAGAGC
CGGTGGCAGCAGGGCAACGTGTTCAGCTGCAGCGTGATGCACGAGGCCCTGC
ACAACCACTACACCCAGAAGTCCCTGAGCCTGAGCCCCGGCAAGCCCTTAATTA
AAttTTGGGTGCTGGTGGTGGTTGGTGGAGTCCTGGCTTGCTATAGCTTGCTAGT
AACAGTGGCCTTTATTATTTTCTGGGTGAGGAGTAAGAGGAGCAGGCTCCTGCA
CAGTGACTACATGAACATGACTCCCCGCCGCCCCGGGCCCACCCGCAAGCATT
ACCAGCCCTATGCCCCACCACGCGACTTCGCAGCCTATCGCTCCAGAGTGAAG
TTCAGCAGGAGCGCAGACGCCCCCGCGTACCAGCAGGGCCAGAACCAGCTCT
ATAACGAGCTCAATCTAGGACGAAGAGAGGAGTACGATGTTTTGGACAAGAGAC
GTGGCCGGGACCCTGAGATGGGGGGAAAGCCGAGAAGGAAGAACCCTCAGGA
AGGCCTGTACAATGAACTGCAGAAAGATAAGATGGCGGAGGCCTACAGTGAGA
TTGGGATGAAAGGCGAGCGCCGGAGGGGCAAGGGGCACGATGGCCTTTACCA
GGGTCTCAGTACAGCCACCAAGGACACCTACGACGCCCTTCACATGCAGGCCC
TGCCTCCTCGCTAA

In one embodiment, the CAR comprises an antigen-binding domain comprising an anti-CDH17 scFv, IgG4 hinge, CD28 transmembrane and co-stimulatory domain, a 4-1BB co-stimulatory domain and a CD3-zeta signalling domain.

In one embodiment, the CAR comprises a protein having at least 75%, 80%, 85%, 90%, 95%, 96%, 97%, 98%, 99% or 100% sequence identity to SEQ ID NO: 47.

Exemplary CAR [A4_4R-IgG4h.28BBz] (SEQ ID NO: 47):
MEAPAQLLFLLLLWLPDTTGDIVMTQSPDSLAVSLGERATINCRSSQSL
LHSSNQRNYLAWYQQKPGQPPKVLIYWASTRESGVPDRFSGSGSGTDFT
LTISSLQAEDVAVYYCQQYYSYPWTFGQGTKVEIKGGGGSGGGGSGGGG
SQVQLVQSGAEVKKPGASVKVSCKASGYTLTDHTIHWMRQAPGQRLEWI
GYIYPRDGITGYNERFRGKATLTADTSASTAYMELSSLRSEDTAVYYCA
RWGYSYRNYAYYYDYWGQGTLVTVSSAAATSSGESKYGPPCPPCPDIFW
VLVVVGGVLACYSLLVTVAFIIFWVRSKRSRLLHSDYMNMTPRRPGPTR
KHYQPYAPPRDFAAYRSASKRGRKKLLYIFKQPFMRPVQTTQEEDGCSC
RFPEEEEGGCELRVKFSRSADAPAYKQGQNQLYNELNLGRREEYDVLDK
RRGRDPEMGGKPRRKNPQEGLYNELQKDKMAEAYSEIGMKGERRRGKGH
DGLYQGLSTATKDTYDALHMQALPPR

In one embodiment, the CAR comprises a protein that is encoded by a nucleotide sequence having at least 75%, 80%, 85%, 90%, 95%, 96%, 97%, 98%, 99% or 100% sequence identity to SEQ ID NO: 51.

Nucleotide sequence encoding exemplary CAR [A4_4R-IgG4h.28BBz]
(SEQ ID NO: 51):
ATGGAGGCCCCCGCCCAGCTGCTGTTCCTGCTGCTGCTGTGGCTGCCCGACAC
CACCGGCGACATCGTGATGACCCAGAGCCCCGACAGCCTGGCCGTGAGCCTG
GGCGAGAGAGCCACCATCAACTGCAGAAGCAGCCAGAGCCTGCTGCACAGCA
GCAACCAGAGAAACTACCTGGCCTGGTACCAGCAGAAGCCCGGCCAGCCCCC
CAAGGTGCTGATCTACTGGGCCAGCACCAGAGAGAGCGGCGTGCCCGACAGA
TTCAGCGGCAGCGGCAGCGGCACCGACTTCACCCTGACCATCAGCAGCCTGCA
GGCCGAGGACGTGGCCGTGTACTACTGCCAGCAGTACTACAGCTACCCCTGGA
CCTTCGGCCAGGGCACCAAGGTGGAGATCAAGGGCGGCGGCGGCAGCGGCG
GCGGCGGCAGCGGCGGCGGCGGCAGCCAGGTGCAGCTGGTGCAGAGCGGCG
CCGAGGTGAAGAAGCCCGGCGCCAGCGTGAAGGTGAGCTGCAAGGCCAGCGG
CTACACCCTGACCGACCACACCATCCACTGGATGAGACAGGCCCCCGGCCAGA
GACTGGAGTGGATCGGCTACATCTACCCCAGAGACGGCATCACCGGCTACAAC
GAGAGATTCAGAGGCAAGGCCACCCTGACCGCCGACACCAGCGCCAGCACCG
CCTACATGGAGCTGAGCAGCCTGAGAAGCGAGGACACCGCCGTGTACTACTGC
GCCAGATGGGGCTACAGCTACAGAAACTACGCCTACTACTACGACTACTGGGG
CCAGGGCACCCTGGTGACCGTGAGCAGCGCCGCCGCCACCAGCAGCGGCGA
GAGCAAGTACGGCCCCCCCTGCCCCCCCTGCCCCGACATCTTCTGGGTGCTGG
TGGTGGTGGGCGGCGTGCTGGCCTGCTACAGCCTGCTGGTGACCGTGGCCTT
CATCATCTTCTGGGTGAGAAGCAAGAGAAGCAGACTGCTGCACAGCGACTACAT
GAACATGACCCCCAGAAGACCCGGCCCCACCAGAAAGCACTACCAGCCCTACG
CCCCCCCCAGAGACTTCGCCGCCTACAGAAGCGCCAGCAAGAGAGGCAGAAA
GAAGCTGCTGTACATCTTCAAGCAGCCCTTCATGAGACCCGTGCAGACCACCCA
GGAGGAGGACGGCTGCAGCTGCAGATTCCCCGAGGAGGAGGAGGGCGGCTG
CGAGCTGAGAGTGAAGTTCAGCAGAAGCGCCGACGCCCCCGCCTACAAGCAG
GGCCAGAACCAGCTGTACAACGAGCTGAACCTGGGCAGAAGAGAGGAGTACGA
CGTGCTGGACAAGAGAAGAGGCAGAGACCCCGAGATGGGCGGCAAGCCCAGA
AGAAAGAACCCCCAGGAGGGCCTGTACAACGAGCTGCAGAAGGACAAGATGGC
CGAGGCCTACAGCGAGATCGGCATGAAGGGCGAGAGAAGAAGAGGCAAGGGC
CACGACGGCCTGTACCAGGGCCTGAGCACCGCCACCAAGGACACCTACGACG
CCCTGCACATGCAGGCCCTGCCCCCCAGATGA

In one embodiment, the CAR comprises a protein that is encoded by a nucleotide sequence having at least 75%, 80%, 85%, 90%, 95%, 96%, 97%, 98%, 99% or 100% sequence identity to SEQ ID NO: 85.

A further nucleotide sequence encoding exemplary CAR [A4_4R-
IgG4h.28BBz] (SEQ ID NO: 85):
ATGGAAGCTCCTGCTCAGCTGCTGTTTCTGCTGCTGCTGTGGCTGCCTGATACC
ACCGGCGACATCGTGATGACACAGAGCCCTGATAGCCTGGCCGTGTCTCTGGG
AGAGAGAGCCACCATCAACTGCAGAAGCAGCCAGAGCCTGCTGCACAGCTCCA
ACCAGAGAAACTACCTGGCCTGGTATCAGCAGAAGCCCGGCCAGCCTCCTAAG
GTGCTGATCTACTGGGCCAGCACCAGAGAATCCGGCGTGCCCGATAGATTTTC
TGGCTCTGGCAGCGGCACCGACTTCACCCTGACAATTTCTAGCCTGCAAGCCG
AGGACGTGGCCGTGTACTACTGCCAGCAGTACTACAGCTACCCCTGGACCTTT
GGCCAGGGCACCAAGGTGGAAATCAAAGGCGGCGGAGGATCTGGCGGAGGTG
GAAGTGGCGGAGGCGGATCTCAAGTTCAGCTGGTTCAGTCTGGCGCCGAAGTG
AAGAAACCTGGCGCCTCTGTGAAGGTGTCCTGCAAGGCCTCTGGCTACACCCT
GACCGATCACACCATCCACTGGATGAGACAGGCCCCTGGCCAGAGACTGGAAT
GGATCGGCTACATCTACCCCAGAGATGGCATCACCGGCTACAACGAGCGGTTC
AGAGGAAAGGCCACACTGACCGCCGATACAAGCGCCAGCACAGCCTACATGGA
ACTGAGCAGCCTGAGAAGCGAGGACACCGCCGTGTATTATTGCGCCAGATGGG
GCTACTCCTACCGGAACTACGCCTACTACTACGACTACTGGGGCCAGGGAACC
CTGGTCACAGTTTCTAGCGCCGCAGCCACAAGCTCCGGCGAGTCTAAATACGG
CCCTCCTTGTCCGCCTTGTCCTGACATCTTTTGGGTGCTCGTGGTCGTTGGCGG
AGTGCTGGCCTGTTATAGCCTGCTGGTTACCGTGGCCTTTATCATCTTCTGGGT
CCGAAGCAAGCGGAGCAGACTGCTGCACTCCGACTACATGAACATGACCCCTA
GACGGCCCGGACCTACCAGAAAGCACTACCAGCCTTACGCTCCTCCTAGAGAC
TTCGCCGCCTACAGAAGCGCCTCCAAGAGAGGCAGAAAGAAGCTGCTGTACAT
CTTCAAGCAGCCCTTCATGCGGCCCGTGCAGACCACACAAGAGGAAGATGGCT
GCAGCTGTCGGTTCCCCGAGGAAGAAGAAGGCGGCTGCGAGCTGAGAGTGAA
GTTCAGCAGATCCGCCGACGCTCCCGCCTATAAGCAGGGACAGAATCAGCTGT
ACAATGAGCTGAACCTGGGGCGCAGAGAAGAGTACGACGTGCTGGACAAGAGA
AGAGGCAGGGACCCTGAGATGGGCGGCAAGCCCAGAAGAAAGAACCCTCAAG
AGGGCCTGTATAACGAGCTGCAGAAAGACAAGATGGCCGAGGCCTACAGCGAG
ATCGGAATGAAGGGCGAACGCCGCAGAGGCAAGGGACACGATGGACTGTATCA
GGGCCTGAGCACCGCCACCAAGGATACCTATGATGCCCTGCACATGCAGGCCC
TGCCACCTAGATAA

In one embodiment, the CAR comprises an antigen-binding domain comprising an anti-CDH17 scFv, LNGFR mutated short (NMS) spacer, CD28 transmembrane and co-stimulatory domain and a CD3-zeta signalling domain.

In one embodiment, the CAR comprises a protein having at least 75%, 80%, 85%, 90%, 95%, 96%, 97%, 98%, 99% or 100% sequence identity to SEQ ID NO: 48.

Exemplary CAR [Lic3-NMS.28z] (SEQ ID NO: 48):
MEAPAQLLFLLLLWLPDTTGQVQLVESGGGVVQPGRSLRLSCAASGFTF
SDYYMYWVRQAPGKGLEWVASISFDGTYTYYTDRVKGRFTISRDNSKNT
LYLQMNSLRAEDTAVYYCARDRPAWFPYWGQGTLVTVSAGGGGSGGGGS
GGGGSGDIVMTQTPLSLSVTPGQPASISCRSSQSIVHSNGNTYLEWYLQ
KPGQSPQLLIYKVSNRFSGVPDRFSGSGSGTDFTLKISRVEAEDVGVYY
CFQGSHVPLTFGAGTKLELKGAPSPVKEACPTGLYTHSGECCKACNLGE
GVAQPCGANQTVCEPCLDSVTFSDVVSATEPCKPCTECVGLQSMSAPCV
EADDAVCRCAYGYYQDETTGRCEACRVCEAGSGLVFSCQDKQNTVCEEC
PDGTYSDEAARAADAECEEPLIKFWVLVVVGGVLACYSLLVTVAFIIFW
VRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALH
MQALPPR

In one embodiment, the CAR comprises a protein that is encoded by a nucleotide sequence having at least 75%, 80%, 85%, 90%, 95%, 96%, 97%, 98%, 99% or 100% sequence identity to SEQ ID NO: 52.

Nucleotide sequence encoding exemplary CAR [Lic3-NMS.28z] (SEQ
ID NO: 52):
ATGGAGGCCCCCGCCCAGCTGCTGTTCCTGCTGCTGCTGTGGCTGCCCGACAC
CACCGGCCAGGTGCAGCTGGTGGAGAGCGGCGGCGGCGTGGTGCAGCCCGG
CAGAAGCCTGAGACTGAGCTGCGCCGCCAGCGGCTTCACCTTCAGCGACTACT
ACATGTACTGGGTGAGACAGGCCCCCGGCAAGGGCCTGGAGTGGGTGGCCAG
CATCAGCTTCGACGGCACCTACACCTACTACACCGACAGAGTGAAGGGCAGAT
TCACCATCAGCAGAGACAACAGCAAGAACACCCTGTACCTGCAGATGAACAGC
CTGAGAGCCGAGGACACCGCCGTGTACTACTGCGCCAGAGACAGACCCGCCT
GGTTCCCCTACTGGGGCCAGGGCACCCTGGTGACCGTGAGCGCCGGCGGCGG
CGGCAGCGGCGGCGGCGGCAGCGGCGGCGGCGGCAGCGGCGACATCGTGAT
GACCCAGACCCCCCTGAGCCTGAGCGTGACCCCCGGCCAGCCCGCCAGCATC
AGCTGCAGAAGCAGCCAGAGCATCGTGCACAGCAACGGCAACACCTACCTGGA
GTGGTACCTGCAGAAGCCCGGCCAGAGCCCCCAGCTGCTGATCTACAAGGTGA
GCAACAGATTCAGCGGCGTGCCCGACAGATTCAGCGGCAGCGGCAGCGGCAC
CGACTTCACCCTGAAGATCAGCAGAGTGGAGGCCGAGGACGTGGGCGTGTACT
ACTGCTTCCAGGGCAGCCACGTGCCCCTGACCTTCGGCGCCGGCACCAAGCT
GGAGCTGAAGGGCGCCCCCTCAccggTCAAAGAGGCCTGCCCCACCGGCCTGT
ACACCCACAGCGGAGAGTGCTGCAAGGCCTGCAACCTGGGAGAGGGCGTGGC
CCAGCCTTGCGGCGCCAATCAGACCGTGTGCGAGCCCTGCCTGGACAGCGTG
ACCTTCAGCGACGTGGTGTCCGCCACCGAGCCCTGCAAGCCTTGCACCGAGTG
TGTGGGCCTGCAGAGCATGAGCGCCCCCTGCGTGGAAGCCGACGACGCCGTG
TGTAGATGCGCCTACGGCTACTACCAGGACGAGACAACCGGCAGATGCGAGGC
CTGTAGAGTGTGCGAGGCCGGCAGCGGCCTGGTGTTCAGTTGTCAGGACAAGC
AGAACACCGTGTGTGAAGAGTGCCCCGACGGCACCTACAGCGACGAGGCCGC
CCGGGCCGCCGACGCCGAGTGCGAGGAACCCTTAATTAAAttTTGGGTGCTGGT
GGTGGTTGGTGGAGTCCTGGCTTGCTATAGCTTGCTAGTAACAGTGGCCTTTAT
TATTTTCTGGGTGAGGAGTAAGAGGAGCAGGCTCCTGCACAGTGACTACATGAA
CATGACTCCCCGCCGCCCCGGGCCCACCCGCAAGCATTACCAGCCCTATGCCC
CACCACGCGACTTCGCAGCCTATCGCTCCAGAGTGAAGTTCAGCAGGAGCGCA
GACGCCCCCGCGTACCAGCAGGGCCAGAACCAGCTCTATAACGAGCTCAATCT
AGGACGAAGAGAGGAGTACGATGTTTTGGACAAGAGACGTGGCCGGGACCCTG
AGATGGGGGGAAAGCCGAGAAGGAAGAACCCTCAGGAAGGCCTGTACAATGAA
CTGCAGAAAGATAAGATGGCGGAGGCCTACAGTGAGATTGGGATGAAAGGCGA
GCGCCGGAGGGGCAAGGGGCACGATGGCCTTTACCAGGGTCTCAGTACAGCC
ACCAAGGACACCTACGACGCCCTTCACATGCAGGCCCTGCCTCCTCGCTAA

In one embodiment, the CAR comprises an antigen-binding domain comprising an anti-CDH17 scFv, LNGFR wildtype long (NWL) spacer, CD28 transmembrane and co-stimulatory domain and a CD3-zeta signalling domain.

In one embodiment, the CAR comprises a protein having at least 75%, 80%, 85%, 90%, 95%, 96%, 97%, 98%, 99% or 100% sequence identity to SEQ ID NO: 49.

Exemplary CAR [Lic3-NWL.28z] (SEQ ID NO: 49):
MEAPAQLLFLLLLWLPDTTGQVQLVESGGGVVQPGRSLRLSCAASGFTF
SDYYMYWVRQAPGKGLEWVASISFDGTYTYYTDRVKGRFTISRDNSKNT
LYLQMNSLRAEDTAVYYCARDRPAWFPYWGQGTLVTVSAGGGGSGGGGS
GGGGSGDIVMTQTPLSLSVTPGQPASISCRSSQSIVHSNGNTYLEWYLQ
KPGQSPQLLIYKVSNRFSGVPDRFSGSGSGTDFTLKISRVEAEDVGVYY
CFQGSHVPLTFGAGTKLELKGAPSPVKEACPTGLYTHSGECCKACNLGE
GVAQPCGANQTVCEPCLDSVTFSDVVSATEPCKPCTECVGLQSMSAPCV
EADDAVCRCAYGYYQDETTGRCEACRVCEAGSGLVFSCQDKQNTVCEEC
PDGTYSDEANHVDPCLPCTVCEDTERQLRECTRWADAECEEIPGRWITR
STPPEGSDSTAPSTQEPEAPPEQDLIASTVAGVVTTVMGSSQPVVTRGT
TDNPLIKFWVLVVVGGVLACYSLLVTVAFIIFWVRSKRSRLLHSDYMNM
TPRRPGPTRKHYQPYAPPRDFAAYRSRVKFSRSADAPAYQQGQNQLYNE
LNLGRREEYDVLDKRRGRDPEMGGKPRRKNPQEGLYNELQKDKMAEAYS
EIGMKGERRRGKGHDGLYQGLSTATKDTYDALHMQALPPR

In one embodiment, the CAR comprises a protein that is encoded by a nucleotide sequence having at least 75%, 80%, 85%, 90%, 95%, 96%, 97%, 98%, 99% or 100% sequence identity to SEQ ID NO: 53.

Nucleotide sequence encoding exemplary CAR [Lic3-NWL.28z] (SEQ
ID NO: 53):
ATGGAGGCCCCCGCCCAGCTGCTGTTCCTGCTGCTGCTGTGGCTGCCCGACAC
CACCGGCCAGGTGCAGCTGGTGGAGAGCGGCGGCGGCGTGGTGCAGCCCGG
CAGAAGCCTGAGACTGAGCTGCGCCGCCAGCGGCTTCACCTTCAGCGACTACT
ACATGTACTGGGTGAGACAGGCCCCCGGCAAGGGCCTGGAGTGGGTGGCCAG
CATCAGCTTCGACGGCACCTACACCTACTACACCGACAGAGTGAAGGGCAGAT
TCACCATCAGCAGAGACAACAGCAAGAACACCCTGTACCTGCAGATGAACAGC
CTGAGAGCCGAGGACACCGCCGTGTACTACTGCGCCAGAGACAGACCCGCCT
GGTTCCCCTACTGGGGCCAGGGCACCCTGGTGACCGTGAGCGCCGGCGGCGG
CGGCAGCGGCGGCGGCGGCAGCGGCGGCGGCGGCAGCGGCGACATCGTGAT
GACCCAGACCCCCCTGAGCCTGAGCGTGACCCCCGGCCAGCCCGCCAGCATC
AGCTGCAGAAGCAGCCAGAGCATCGTGCACAGCAACGGCAACACCTACCTGGA
GTGGTACCTGCAGAAGCCCGGCCAGAGCCCCCAGCTGCTGATCTACAAGGTGA
GCAACAGATTCAGCGGCGTGCCCGACAGATTCAGCGGCAGCGGCAGCGGCAC
CGACTTCACCCTGAAGATCAGCAGAGTGGAGGCCGAGGACGTGGGCGTGTACT
ACTGCTTCCAGGGCAGCCACGTGCCCCTGACCTTCGGCGCCGGCACCAAGCT
GGAGCTGAAGGGCGCCCCCTCAccggTCAAAGAGGCCTGCCCCACCGGCCTGT
ACACCCACAGCGGAGAGTGCTGCAAGGCCTGCAACCTGGGAGAGGGCGTGGC
CCAGCCTTGCGGCGCCAATCAGACCGTGTGCGAGCCCTGCCTGGACAGCGTG
ACCTTCAGCGACGTGGTGTCCGCCACCGAGCCCTGCAAGCCTTGCACCGAGTG
TGTGGGCCTGCAGAGCATGAGCGCCCCCTGCGTGGAAGCCGACGACGCCGTG
TGTAGATGCGCCTACGGCTACTACCAGGACGAGACAACCGGCAGATGCGAGGC
CTGTAGAGTGTGCGAGGCCGGCAGCGGCCTGGTGTTCAGTTGTCAAGACAAGC
AGAATACCGTGTGTGAAGAGTGCCCCGACGGCACCTACAGCGACGAGGCCAAC
CACGTGGACCCCTGCCTGCCCTGCACTGTGTGCGAGGACACCGAGCGGCAGC
TGCGCGAGTGCACAAGATGGGCCGACGCCGAGTGCGAAGAGATCCCCGGCAG
ATGGATCACCAGAAGCACCCCCCCTGAGGGCAGCGACAGCACCGCCCCTAGC
ACCCAGGAACCTGAGGCCCCTCCCGAGCAGGACCTGATCGCCTCTACAGTGGC
CGGCGTGGTGACAACCGTGATGGGCAGCTCTCAGCCCGTGGTGACACGGGGC
ACCACCGACAATCCCTTAATTAAattTTGGGTGCTGGTGGTGGTTGGTGGAGTCC
TGGCTTGCTATAGCTTGCTAGTAACAGTGGCCTTTATTATTTTCTGGGTGAGGAG
TAAGAGGAGCAGGCTCCTGCACAGTGACTACATGAACATGACTCCCCGCCGCC
CCGGGCCCACCCGCAAGCATTACCAGCCCTATGCCCCACCACGCGACTTCGCA
GCCTATCGCTCCAGAGTGAAGTTCAGCAGGAGCGCAGACGCCCCCGCGTACCA
GCAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAAGA
TGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACG
ACGCCCTTCACATGCAGGCCCTGCCTCCTCGCTAA

In one embodiment, the CAR comprises an antigen-binding domain comprising an anti-CDH17 scFv, IgG1 hinge, CD28 transmembrane and co-stimulatory domain and a CD3-zeta signalling domain.

In one embodiment, the CAR comprises a protein having at least 75%, 80%, 85%, 90%, 95%, 96%, 97%, 98%, 99% or 100% sequence identity to SEQ ID NO: 50.

Exemplary CAR [Lic3-hinge.28z] (SEQ ID NO: 50):
MEAPAQLLFLLLLWLPDTTGQVQLVESGGGVVQPGRSLRLSCAASGFTF
SDYYMYWVRQAPGKGLEWVASISFDGTYTYYTDRVKGRFTISRDNSKNT
LYLQMNSLRAEDTAVYYCARDRPAWFPYWGQGTLVTVSAGGGGSGGGGS
GGGGSGDIVMTQTPLSLSVTPGQPASISCRSSQSIVHSNGNTYLEWYLQ
KPGQSPQLLIYKVSNRFSGVPDRFSGSGSGTDFTLKISRVEAEDVGVYY
CFQGSHVPLTFGAGTKLELKGAPSPVEPKSCDKTHTCPPCPPLIKFWVL
VVVGGVLACYSLLVTVAFIIFWVRSKRSRLLHSDYMNMTPRRPGPTRKH
YQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRG
KGHDGLYQGLSTATKDTYDALHMQALPPR

In one embodiment, the CAR comprises a protein that is encoded by a nucleotide sequence having at least 75%, 80%, 85%, 90%, 95%, 96%, 97%, 98%, 99% or 100% sequence identity to SEQ ID NO: 54.

Nucleotide sequence encoding exemplary CAR [Lic3-
hinge.28z] (SEQ ID NO: 54):
ATGGAGGCCCCCGCCCAGCTGCTGTTCCTGCTGCTGCTGTGGCTGCCCG
ACACCACCGGCCAGGTGCAGCTGGTGGAGAGCGGCGGCGGCGTGGTGCA
GCCCGGCAGAAGCCTGAGACTGAGCTGCGCCGCCAGCGGCTTCACCTTC
AGCGACTACTACATGTACTGGGTGAGACAGGCCCCCGGCAAGGGCCTGG
AGTGGGTGGCCAGCATCAGCTTCGACGGCACCTACACCTACTACACCGA
CAGAGTGAAGGGCAGATTCACCATCAGCAGAGACAACAGCAAGAACACC
CTGTACCTGCAGATGAACAGCCTGAGAGCCGAGGACACCGCCGTGTACT
ACTGCGCCAGAGACAGACCCGCCTGGTTCCCCTACTGGGGCCAGGGCAC
CCTGGTGACCGTGAGCGCCGGCGGCGGCGGCAGCGGCGGCGGCGGCAGC
GGCGGCGGCGGCAGCGGCGACATCGTGATGACCCAGACCCCCCTGAGCC
TGAGCGTGACCCCCGGCCAGCCCGCCAGCATCAGCTGCAGAAGCAGCCA
GAGCATCGTGCACAGCAACGGCAACACCTACCTGGAGTGGTACCTGCAG
AAGCCCGGCCAGAGCCCCCAGCTGCTGATCTACAAGGTGAGCAACAGAT
TCAGCGGCGTGCCCGACAGATTCAGCGGCAGCGGCAGCGGCACCGACTT
CACCCTGAAGATCAGCAGAGTGGAGGCCGAGGACGTGGGCGTGTACTAC
TGCTTCCAGGGCAGCCACGTGCCCCTGACCTTCGGCGCCGGCACCAAGC
TGGAGCTGAAGGGCGCCCCCTCAccggTCGAGCCCAAGAGCTGCGACAA
GACCCACACCTGTCCCCCCTGCCCCCCCTTAatTAAAttTTGGGTGCTG
GTGGTGGTTGGTGGAGTCCTGGCTTGCTATAGCTTGCTAGTAACAGTGG
CCTTTATTATTTTCTGGGTGAGGAGTAAGAGGAGCAGGCTCCTGCACAG
TGACTACATGAACATGACTCCCCGCCGCCCCGGGCCCACCCGCAAGCAT
TACCAGCCCTATGCCCCACCACGCGACTTCGCAGCCTATCGCTCCAGAG
TGAAGTTCAGCAGGAGCGCAGACGCCCCCGCGTACCAGCAGGGCCAGAA
CCAGCTCTATAACGAGCTCAATCTAGGACGAAGAGAGGAGTACGATGTT
TTGGACAAGAGACGTGGCCGGGACCCTGAGATGGGGGGAAAGCCGAGAA
GGAAGAACCCTCAGGAAGGCCTGTACAATGAACTGCAGAAAGATAAGAT
GGCGGAGGCCTACAGTGAGATTGGGATGAAAGGCGAGCGCCGGAGGGGC
AAGGGGCACGATGGCCTTTACCAGGGTCTCAGTACAGCCACCAAGGACA
CCTACGACGCCCTTCACATGCAGGCCCTGCCTCCTCGCTAA

In one embodiment, the CAR comprises a sequence with at least 90% sequence identity to the sequence of any one of SEQ ID NOs: 9, 14, 15, 23, 24, 45 or 47-50.

In one embodiment, the CAR consists of a sequence with at least 90% sequence identity to the sequence of any one of SEQ ID NOs: 14, 15, 23, 24, or 47-50.

In one embodiment, the CAR comprises the sequence of any one of SEQ ID NOs: 9, 14, 15, 23, 24, 45 or 47-50.

In one embodiment, the CAR comprises a sequence with at least 90% sequence identity to the sequence of any one of SEQ ID NOs: 9, 14, 15, 23, 24, 45 or 47-50, wherein said sequences comprise any one or more of the sequences according to SEQ ID NOs: 1-6 or 39-44.

In one embodiment, the CAR comprises a sequence with at least 90% sequence identity to the sequence of any one of SEQ ID NOs: 9, 14, 15, 23, 24, 45 or 47-50, wherein said sequences comprise the sequences according to SEQ ID NOs: 1-6 or 39-44.

In one embodiment, the CAR comprises a sequence with at least 90% sequence identity to the sequence of any one of SEQ ID NOs: 45 or 47-50.

In one embodiment, the CAR consists of a sequence with at least 90% sequence identity to the sequence of any one of SEQ ID NOs: 47-50.

In one embodiment, the CAR comprises the sequence of any one of SEQ ID NOs: 45 or 47-50.

In one embodiment, the CAR comprises a sequence with at least 90% sequence identity to the sequence of any one of SEQ ID NOs: 45 or 47-50, wherein said sequences comprise any one or more of the sequences according to SEQ ID NOs: 39-44.

In one embodiment, the CAR comprises a sequence with at least 90% sequence identity to the sequence of any one of SEQ ID NOs: 45 or 47-50, wherein said sequences comprise the sequences according to SEQ ID NOs: 39-44.

### Tags and Linkers

The polynucleotide and polypeptide sequences of the invention may further comprise tag and/or linker sequences.

### Linkers

At both the polynucleotide and polypeptide levels, certain functional sequence elements may be separated by linkers. Linkers typically comprise a short polynucleotide or polypeptide sequence. Linkers may be used to physically separate functional sequences in order, e.g., to improve the functionality of said sequences.

In one embodiment, the polynucleotide of the invention comprises one or more linker sequences.

In one embodiment, the sequence encoding a functional domain of the CAR is separated from another sequence encoding a functional domain by a linker.

In one embodiment, the CAR of the invention comprises one or more linker sequences.

In one embodiment, the linker is a GS linker, or a GGS linker.

GGS linkers may be made up of GGS repeats, such as 1, 2, 3, 4, 5, 6, 7, 8, 9, 10, or more than 10 GGS repeats. GGS linkers may comprise more than 10 repeats of the amino acid sequence GGS, such as more than 20, more than 30, or more than 40 repeats.

GGS linkers may comprise variations of the GGS sequence, such as GGGS (SEQ ID NO: 62); GGGGS (SEQ ID NO: 63). Furthermore, GGS motifs and variants thereof may also comprise incomplete motifs, e.g., GS.

Exemplary GGS repeat containing linker sequences are illustrated as SEQ ID NOs: 64 and 65

(SEQ ID NO: 64)
GGGGSGGGGSGGGGSG
(SEQ ID NO: 65)
GGGGSGGGGSGGGGS

GGS linkers such as those comprising or consisting of any one of SEQ ID NOs: 62-65 may be placed between other functional sequences, such as VH and VL sequences within an scFV, antigen-specific binding domain, or CAR.

### Tags

Both the polynucleotides and polypeptides of the invention may comprise tags. Tags are typically sequences that facilitate the detection or isolation of the molecule to which they are attached. Tags may be particularly useful in experimental studies utilising the polynucleotides or polypeptides of the invention.

In one embodiment, the polynucleotide of the invention comprises one or more tag sequences.

In one embodiment, the CAR or scFv of the invention comprises one or more tag sequences.

### Further Polypeptides or Polynucleotides of Interest

In addition to the CAR of the invention, further polypeptides or polynucleotides of interest may be provided. Such polypeptides or polynucleotides of interest may be provided alongside the CAR or polynucleotide(s) encoding therefor, for example, within the same molecule or vector, or within a distinct molecule or vector.

In some embodiments, the polynucleotide of interest encodes a polypeptide of interest.

In one aspect, the invention provides a product (e.g. a composition or a kit) comprising the CAR of the invention and a polypeptide of interest.

In some embodiments, the polynucleotide of the invention further comprises a nucleotide sequence encoding a polypeptide of interest.

In some embodiments, the cell of the invention further comprises a polypeptide of interest or a polynucleotide encoding therefor.

The polypeptides or polynucleotides of interest may be used for various purposes, e.g., selection of successfully transduced cells, increasing the effectiveness of the CAR-containing cell, or providing a mechanism of CAR cell inactivation.

### Immunomodulatory Molecules

The polypeptide of interest may be an immunomodulatory molecule. Such molecules may, for example, stimulate an immune response and increase the efficacy of a cell comprising a CAR according to the invention.

In one aspect, there is provided a polynucleotide comprising (a) one or more nucleotide sequence encoding the chimeric antigen receptor (CAR) of the invention; and (b) a nucleotide sequence encoding an immunomodulatory molecule (e.g. a cytokine).

In one aspect, there is provided a product comprising (a) a first polynucleotide comprising one or more nucleotide sequence encoding the chimeric antigen receptor (CAR) of the invention;

and (b) a second polynucleotide comprising a nucleotide sequence encoding an immunomodulatory molecule (e.g. a cytokine). The product may be, for example, a kit or a composition. In one aspect, there is provided a kit comprising (a) a first polynucleotide comprising one or more nucleotide sequence encoding the chimeric antigen receptor (CAR) of the invention; and (b) a second polynucleotide comprising a nucleotide sequence encoding an immunomodulatory molecule (e.g. a cytokine). In another aspect, there is provided a composition comprising (a) a first polynucleotide comprising one or more nucleotide sequence encoding the chimeric antigen receptor (CAR) of the invention; and (b) a second polynucleotide comprising a nucleotide sequence encoding an immunomodulatory molecule (e.g. a cytokine).

In one embodiment, the polypeptide of interest is a cytokine.

In one embodiment, the cytokine is IL-12.

### Selection Markers & Suicide Genes

Additional genes or polynucleotides may be provided alongside the CARs and scFvs of the invention.

Genes may also be provided that allow for the selection of engineered cells, such as cells comprising the CAR according to the present invention, e.g., in vitro or in vivo.

In one aspect, there is provided a polynucleotide comprising (a) one or more nucleotide sequence encoding the chimeric antigen receptor (CAR) of the invention; and (b) a nucleotide sequence encoding a selection marker or a suicide gene.

In one aspect, there is provided a product comprising (a) a first polynucleotide comprising one or more nucleotide sequence encoding the chimeric antigen receptor (CAR) of the invention; and (b) a second polynucleotide comprising a nucleotide sequence encoding a selection marker or a suicide gene. The product may be, for example, a kit or a composition. In one aspect, there is provided a kit comprising (a) a first polynucleotide comprising one or more nucleotide sequence encoding the chimeric antigen receptor (CAR) of the invention; and (b) a second polynucleotide comprising a nucleotide sequence encoding a selection marker or a suicide gene. In another aspect, there is provided a composition comprising (a) a first polynucleotide comprising one or more nucleotide sequence encoding the chimeric antigen receptor (CAR) of the invention; and (b) a second polynucleotide comprising a nucleotide sequence encoding a selection marker or a suicide gene.

In one embodiment, the polypeptide of interest is a selection marker.

In one embodiment, the polynucleotide of interest encodes a selection marker.

Human cell surface proteins, such as ΔNGFR, CD34, CD19, CD20 and CD4, and CD90, have been used as surrogate markers for the identification of ex vivo genetically modified cells. In one embodiment, the polypeptide of interest is any one or more of ΔNGFR, CD34, CD19, CD20 and CD4, and CD90.

In one embodiment the selection marker is thymidine kinase (TK), or a modified version thereof.

An example TK amino acid sequence is (SEQ ID NO: 80; TK mut2):
MASYPCHQHASAFDQAARSRGHSNRRTALRPRRQQEATEVRLEQKMPTLLRVYID
GPHGMGKTTTTQLLVALGSRDDIVYVPEPMTYWQVLGASETIANIYTTQHRLDQGEI
SAGDAAVVMTSAQITMGMPYAVTDAVLAPHVGGEAGSSHAPPPALTLIFDRHPIAAL
LCYPAARYLMGSMTPQAVLAFVALIPPTLPGTNIVLGALPEDRHIDRLAKRQRPGER
LDLAMLAAIRRVYGLLANTVRYLQGGGSWWEDWGQLSGTAVPPQGAEPQSNAGP
RPHIGDTLFTLFRAPELLAPNGDLYNVFAWALDVLAKRLRPMHVFILDYDQSPAGCR
DALLQLTSGMVQTHVTTPGSIPTICDLARTFAREMGEAN
An example nucleotide sequence encoding TK is (SEQ ID NO: 81):
ATGGCTTCGTACCCCTGCCATCAACACGCGTCTGCGTTCGACCAGGCTGCGCG
TTCTCGCGGCCATAGCAACCGACGTACGGCGTTGCGCCCTCGCCGGCAGCAA
GAAGCCACGGAAGTCCGCCTGGAGCAGAAAATGCCCACGCTACTGCGGGTTTA
TATAGACGGTCCTCACGGGATGGGGAAAACCACCACCACGCAACTGCTGGTGG
CCCTGGGTTCGCGCGACGATATCGTCTACGTACCCGAGCCGATGACTTACTGG
CAGGTGCTGGGGGCTTCCGAGACAATCGCGAACATCTACACCACACAACACCG
CCTCGACCAGGGCGAGATATCGGCCGGGGACGCGGCGGTGGTAATGACAAGC
GCCCAGATAACAATGGGCATGCCTTATGCCGTGACCGACGCCGTTCTGGCTCC
TCATGTCGGGGGGGAGGCTGGGAGTTCACATGCCCCGCCCCCGGCCCTCACC
CTCATCTTCGACCGCCATCCCATCGCCGCCCTCCTGTGCTACCCGGCCGCGCG
ATACCTTATGGGCAGCATGACCCCCCAGGCCGTGCTGGCGTTCGTGGCCCTCA
TCCCGCCGACCTTGCCCGGCACAAACATCGTGTTGGGGGCCCTTCCGGAGGAC
AGACACATCGACCGCCTGGCCAAACGCCAGCGCCCCGGCGAGCGGCTTGACC
TGGCTATGCTGGCCGCGATTCGCCGCGTTTACGGGCTGCTTGCCAATACGGTG
CGGTATCTGCAGGGCGGCGGGTCGTGGTGGGAGGATTGGGGACAGCTTTCGG
GGACGGCCGTGCCGCCCCAGGGTGCCGAGCCCCAGAGCAACGCGGGCCCAC
GACCCCATATCGGGGACACGTTATTTACCCTGTTTCGGGCCCCCGAGTTGCTG
GCCCCCAACGGCGACCTGTATAACGTGTTTGCCTGGGCCTTGGACGTCTTGGC
CAAACGCCTCCGTCCCATGCACGTCTTTATCCTGGATTACGACCAATCGCCCGC
CGGCTGCCGGGACGCCCTGCTGCAACTTACCTCCGGGATGGTCCAGACCCAC
GTCACCACCCCAGGCTCCATACCGACGATCTGCGACCTGGCGCGCACGTTTGC
CCGGGAGATGGGGGAGGCTAACTGA
An example nucleotide sequence encoding TK is (SEQ ID NO: 82):
ATGGCCAGCTATCCTTGTCACCAGCACGCCAGCGCCTTTGATCAGGCCGCAAG
ATCTAGAGGCCACAGCAACAGAAGAACAGCCCTGCGGCCTCGGAGACAGCAAG
AGGCTACAGAAGTTCGGCTGGAACAGAAGATGCCCACACTGCTGCGGGTGTAC
ATCGATGGCCCTCACGGCATGGGCAAGACCACCACAACACAGCTGCTGGTGGC
CCTGGGCAGCAGAGATGACATCGTGTATGTGCCCGAGCCTATGACCTACTGGC
AGGTTCTGGGAGCCAGCGAGACAATCGCCAACATCTACACCACACAGCACCGG
CTGGATCAGGGCGAAATTTCTGCTGGCGACGCCGCCGTGGTTATGACATCTGC
CCAGATCACCATGGGCATGCCTTACGCCGTGACAGATGCTGTGCTGGCCCCTC
ATGTTGGCGGAGAAGCCGGATCTTCTCATGCCCCTCCACCAGCTCTGACCCTG
ATCTTCGACAGACACCCTATCGCCGCTCTGCTGTGTTATCCTGCCGCCAGATAC
CTGATGGGCAGCATGACACCTCAGGCCGTGCTGGCTTTCGTGGCCCTGATTCC
TCCTACACTGCCCGGCACCAATATCGTGCTGGGAGCCCTGCCTGAGGACCGGC
ACATTGATAGACTGGCCAAGAGACAGCGGCCTGGCGAGAGACTGGATCTGGCT
ATGCTGGCCGCCATCAGAAGAGTGTACGGCCTGCTGGCCAACACCGTGCGGTA
TCTTCAAGGCGGAGGATCTTGGTGGGAAGATTGGGGCCAGCTGTCTGGCACAG
CAGTTCCTCCACAAGGCGCCGAGCCTCAGTCTAATGCTGGACCCAGACCTCAC
ATCGGCGACACCCTGTTTACCCTGTTCAGAGCCCCTGAGCTGCTGGCTCCTAAC
GGCGACCTGTACAACGTGTTCGCCTGGGCTCTTGACGTGCTGGCAAAAAGACT
GCGGCCCATGCACGTGTTCATCCTGGACTACGATCAGTCCCCTGCCGGCTGTA
GAGATGCTCTGCTGCAGCTGACAAGCGGCATGGTGCAGACCCACGTTACAACC
CCTGGCAGCATCCCCACCATCTGTGACCTGGCCAGAACCTTCGCCAGAGAGAT
GGGCGAAGCCAACTGA

In some embodiments the TK comprises an amino acid sequence having at least 70%, 75%, 80%, 85%, 90%, 95%, 96%, 97%, 98%, 99%, or 100% sequence identity to SEQ ID NO: 80. In some embodiments the TK consists of an amino acid sequence having at least 70%, 75%, 80%, 85%, 90%, 95%, 96%, 97%, 98%, 99%, or 100% sequence identity to SEQ ID NO: 80.

In some embodiments the TK is encoded by a nucleotide sequence having at least 70%, 75%, 80%, 85%, 90%, 95%, 96%, 97%, 98%, 99%, or 100% sequence identity to SEQ ID NO: 81 or 82.

In one embodiment, the polypeptide of interest is a functionally inert truncated version of human epidermal growth factor receptor (EGFRt) or an enhanced version of EGFRt (eEGFRt), as described in WO2021/229075.

An example EGFRt amino acid sequence is (SEQ ID NO: 55):
RKVCNGIGIGEFKDSLSINATNIKHFKNCTSISGDLHILPVAFRGDSFTHTPPLDPQEL
DILKTVKEITGFLLIQAWPENRTDLHAFENLEIIRGRTKQHGQFSLAVVSLNITSLGLR
SLKEISDGDVIISGNKNLCYANTINWKKLFGTSGQKTKIISNRGENSCKATGQVCHAL
CSPEGCWGPEPRDCVSCRNVSRGRECVDKCNLLEGEPREFVENSECIQCHPECL
PQAMNITCTGRGPDNCIQCAHYIDGPHCVKTCPAGVMGENNTLVWKYADAGHVCH
LCHPNCTYGCTGPGLEGCPTNGPKIPSIATGMVGALLLLLVVALGIGLFM
An example nucleotide sequence encoding EGFRt is (SEQ ID NO: 56):
AGAAAAGTGTGCAACGGCATCGGCATCGGAGAGTTCAAGGACAGCCTGAGCAT
CAACGCCACCAACATCAAGCACTTCAAGAACTGCACCAGCATCAGCGGCGACC
TGCACATTCTGCCTGTGGCCTTTAGAGGCGACAGCTTCACCCACACACCTCCAC
TGGATCCCCAAGAGCTGGACATCCTGAAAACCGTGAAAGAGATCACCGGATTTC
TGTTGATCCAGGCTTGGCCCGAGAACCGGACAGATCTGCACGCCTTCGAGAAC
CTGGAAATCATCAGAGGCCGGACCAAGCAGCACGGCCAGTTTTCTCTGGCTGT
GGTGTCCCTGAACATCACCAGCCTGGGCCTGAGAAGCCTGAAAGAAATCAGCG
ACGGCGACGTGATCATCTCCGGCAACAAGAACCTGTGCTACGCCAACACCATC
AACTGGAAGAAGCTGTTCGGCACCAGCGGCCAGAAAACAAAGATCATCAGCAA
CCGGGGCGAGAACAGCTGCAAGGCTACAGGCCAAGTGTGCCACGCTCTGTGTA
GCCCTGAAGGCTGTTGGGGACCCGAGCCTAGAGATTGCGTGTCCTGCAGAAAC
GTGTCCCGGGGCAGAGAATGCGTGGACAAGTGCAATCTGCTGGAAGGCGAGC
CCCGCGAGTTCGTGGAAAACAGCGAGTGCATCCAGTGTCACCCCGAGTGTCTG
CCCCAGGCCATGAACATTACCTGTACCGGCAGAGGCCCCGACAACTGTATTCA
GTGCGCCCACTACATCGACGGCCCTCACTGCGTGAAAACATGTCCTGCTGGCG
TGATGGGAGAGAACAACACCCTCGTGTGGAAGTATGCCGACGCCGGACATGTG
TGCCACCTGTGTCACCCTAATTGCACCTACGGCTGTACAGGCCCTGGCCTGGA
AGGCTGTCCAACAAACGGACCTAAGATCCCCTCTATCGCCACCGGCATGGTTG
GAGCCCTGCTGCTTCTGCTGGTGGTGGCCCTTGGAATCGGCCTGTTTATG

In some embodiments the EGFRt comprises an amino acid sequence having at least 70%, 75%, 80%, 85%, 90%, 95%, 96%, 97%, 98%, 99% or 100% sequence identity to SEQ ID NO: 55. In some embodiments the EGFRt consists of an amino acid sequence having at least 70%, 75%, 80%, 85%, 90%, 95%, 96%, 97%, 98%, 99% or 100% sequence identity to SEQ ID NO: 55. Preferably the EGFRt comprises an epitope recognisable by an antibody, such as cetuximab. Preferably the EGFRt lacks signalling or trafficking activity.

In one embodiment, the selection marker is the human epidermal growth factor receptor or a modified version thereof.

In one embodiment, the selection marker is a truncated human epidermal growth factor receptor (EGFRt), or a modified version thereof.

In one embodiment, the selection marker is a truncated human epidermal growth factor receptor (EGFRt) encoded by SEQ ID NO: 56, or a sequence with at least 75% sequence identity thereto.

In one embodiment, the selection marker is an enhanced truncated human epidermal growth factor receptor (eEGFRt), or a modified version thereof.

In one embodiment, the selection marker is CD20.

In one embodiment, the polynucleotide or vector comprises one or more promoter(s), operably linked to the nucleotide sequence encoding CD20.

An example CD20 sequence is (SEQ ID NO: 70):
MTTPRNSVNGTFPAEPMKGPIAMQSGPKPLFRRMSSLVGPTQSFFMRES
KTLGAVQIMNGLFHIALGGLLMIPAGIYAPICVTVWYPLWGGIMYIISG
SLLAATEKNSRKCLVKGKMIMNSLSLFAAISGMILSIMDILNIKISHFL
KMESLNFIRAHTPYINIYNCEPANPSEKNSPSTQYCYSIQSLFLGILSV
MLIFAFFQELVIAGIVENEWKRTCSRPKSNIVLLSAEEKKEQTIEIKEE
VVGLTETSSQPKNEEDIEIIPIQEEEEEETETNFPEPPQDQESSPIEND
SSP

In some embodiments the CD20 comprises an amino acid sequence having at least 70%, 75%, 80%, 85%, 90%, 95%, 96%, 97%, 98%, 99%, or 100% sequence identity to SEQ ID NO: 70. In some embodiments the CD20 consists of an amino acid sequence having at least 70%, 75%, 80%, 85%, 90%, 95%, 96%, 97%, 98%, 99%, or 100% sequence identity to SEQ ID NO: 70.

In one embodiment, the selection marker is NGFR.

Suicide genes act as a means to selectively induce the elimination of cells that contain them, in order to provide a safety mechanism that mitigates against possible adverse effects of adoptive cell therapies, such as severe autoimmunity or graft-versus-host disease.

In one embodiment, the polynucleotide of interest is a suicide gene.

Advantageously, a polynucleotide of interest may act as both a selection marker and a suicide gene.

In a preferred embodiment the polynucleotide of interest acts as a suicide gene and a selection marker.

In one embodiment, the suicide gene is CD20.

In one embodiment, the suicide gene is thymidine kinase (TK), or a modified version thereof.

In one embodiment, the suicide gene is human epidermal growth factor receptor, or a modified version thereof.

In one embodiment, the suicide gene is truncated human epidermal growth factor receptor (EGFRt) or an enhanced truncated human epidermal growth factor receptor (EGFRt).

In one embodiment, the suicide gene is caspase.

An example caspase amino acid sequence is (SEQ ID NO: 83):
MLEGVQVETISPGDGRTFPKRGQTCVVHYTGMLEDGKKVDSSRDRNKPFKFMLGK
QEVIRGWEEGVAQMSVGQRAKLTISPDYAYGATGHPGIIPPHATLVFDVELLKLESG
GGSGVDGFGDVGALESLRGNADLAYILSMEPCGHCLIINNVNFCRESGLRTRTGSNI
DCEKLRRRFSSLHFMVEVKGDLTAKKMVLALLELAQQDHGALDCCVVVILSHGCQA
SHLQFPGAVYGTDGCPVSVEKIVNIFNGTSCPSLGGKPKLFFIQACGGEQKDHGFE
VASTSPEDESPGSNPEPDATPFQEGLRTFDQLDAISSLPTPSDIFVSYSTFPGFVSW
RDPKSGSWYVETLDDIFEQWAHSEDLQSLLLRVANAVSVKGIYKQMPGCFNFLRKK
LFFKTSASRA
An example nucleotide sequence encoding caspase is (SEQ ID NO: 84):
ATGCTGGAAGGCGTGCAGGTCGAGACAATCTCTCCTGGCGACGGCAGAACATT
CCCCAAGAGGGGCCAGACATGCGTGGTGCACTATACCGGCATGCTCGAGGAC
GGCAAGAAGGTGGACAGCAGCCGGGACAGAAACAAGCCCTTCAAGTTCATGCT
GGGCAAGCAAGAAGTGATCAGAGGCTGGGAAGAGGGCGTCGCCCAGATGTCT
GTTGGACAGAGAGCCAAGCTGACAATCAGCCCCGATTACGCCTATGGCGCCAC
AGGACACCCTGGCATCATTCCTCCACATGCCACACTGGTGTTCGACGTGGAACT
GCTGAAGCTGGAAAGCGGCGGAGGATCTGGCGTCGACGGATTTGGAGATGTG
GGCGCCCTGGAAAGCCTGAGAGGAAATGCCGATCTGGCCTACATCCTGAGCAT
GGAACCTTGCGGCCACTGCCTGATTATCAACAACGTGAACTTCTGCAGAGAGAG
CGGCCTGAGAACCAGAACCGGCAGCAACATCGACTGCGAGAAGCTGCGGAGA
AGATTCAGCAGCCTGCACTTCATGGTGGAAGTGAAGGGCGACCTGACCGCCAA
GAAAATGGTGCTGGCTCTGCTGGAACTGGCCCAGCAAGATCATGGCGCTCTGG
ATTGCTGCGTGGTCGTGATCCTGTCTCATGGCTGTCAGGCCAGCCATCTGCAAT
TCCCTGGCGCCGTGTATGGCACCGATGGCTGTCCTGTGTCCGTGGAAAAGATC
GTGAACATCTTCAACGGCACCAGCTGTCCTAGCCTCGGCGGAAAGCCCAAGCT
GTTCTTCATCCAAGCCTGTGGCGGCGAGCAGAAGGATCACGGATTTGAGGTGG
CCAGCACAAGCCCCGAGGATGAGTCTCCTGGAAGCAACCCTGAGCCTGACGCC
ACACCTTTCCAAGAGGGACTGAGAACCTTCGACCAGCTGGACGCTATCAGCTC
CCTGCCTACACCTAGCGACATCTTCGTGTCCTACAGCACATTCCCCGGCTTTGT
GTCTTGGCGGGATCCCAAGTCTGGCTCTTGGTACGTGGAAACCCTGGACGACA
TCTTTGAGCAGTGGGCCCATAGCGAGGACCTGCAGTCTCTGCTGCTGAGAGTG
GCCAATGCCGTGTCCGTGAAGGGCATCTACAAGCAGATGCCTGGCTGCTTCAA
CTTCCTGCGGAAGAAGCTGTTTTTCAAGACCAGCGCTAGCCGGGCCTGA

In some embodiments the caspase comprises an amino acid sequence having at least 70%, 75%, 80%, 85%, 90%, 95%, 96%, 97%, 98%, 99%, or 100% sequence identity to SEQ ID NO: 83. In some embodiments the caspase consists of an amino acid sequence having at least 70%, 75%, 80%, 85%, 90%, 95%, 96%, 97%, 98%, 99%, or 100% sequence identity to SEQ ID NO: 83.

In some embodiments the caspase is encoded by a nucleotide sequence having at least 70%, 75%, 80%, 85%, 90%, 95%, 96%, 97%, 98%, 99%, or 100% sequence identity to SEQ ID NO: 84.

In one embodiment, the polynucleotide or vector comprises a minimal core promoter derived from cytomegalovirus (minCMV), operably linked to the nucleotide sequence encoding CD20.

In one embodiment, the nucleotide sequence encoding CD20 is encoded in the antisense orientation relative to the nucleotide sequence encoding the CAR.

In one embodiment, the polynucleotide or vector comprises a nucleotide sequence encoding the CAR and a nucleotide sequence encoding CD20.

In one embodiment, the polynucleotide or vector comprises a human phosphoglycerate kinase (PGK) promoter operably linked to the nucleotide sequence encoding the CAR and a minimal core promoter derived from cytomegalovirus (minCMV), operably linked to the nucleotide sequence encoding CD20.

In one embodiment, CD20 and the CAR are encoded in opposing directions within a polynucleotide or vector.

It will be understood that, where appropriate, any of the embodiments described for the selection markers may apply to instances where said selection marker is also functioning as a suicide gene, and vice versa.

### Glycosidases

Additional polypeptides or polynucleotides may be provided alongside the CARs and scFvs of the invention. In one embodiment, the polypeptide of interest is a glycosidase.

In one aspect, there is provided a polynucleotide comprising (a) one or more nucleotide sequence encoding the chimeric antigen receptor (CAR) of the invention; and (b) a nucleotide sequence encoding a glycosidase.

In one aspect, there is provided a product comprising (a) a first polynucleotide comprising one or more nucleotide sequence encoding the chimeric antigen receptor (CAR) of the invention; and (b) a second polynucleotide comprising a nucleotide sequence encoding a glycosidase. The product may be, for example, a kit or a composition. In one aspect, there is provided a kit comprising (a) a first polynucleotide comprising one or more nucleotide sequence encoding the chimeric antigen receptor (CAR) of the invention; and (b) a second polynucleotide comprising a nucleotide sequence encoding a glycosidase. In another aspect, there is provided a composition comprising (a) a first polynucleotide comprising one or more nucleotide sequence encoding the chimeric antigen receptor (CAR) of the invention; and (b) a second polynucleotide comprising a nucleotide sequence encoding a glycosidase.

Glycosidases (or glycoside hydrolases/glycosyl hydrolases) are enzymes that catalyse the hydrolysis of glycosidic bonds in sugars. Glycosidases that act upon carbohydrates that are bound to proteins, e.g., as N- or O-linked glycans, may also be referred to herein as deglycosylating enzymes as they catalyse removal of glycans or sugar moieties.

Removal of glycans or sugars from extracellular proteins, or the degradation of extracellular glycans, may facilitate target recognition by CARs and thereby enhance their activity.

Glycosidases that are functional in the extracellular environment may catalyze the removal of glycans (or “act upon” glycans) from diverse substrates. In a preferred embodiment, the glycosidase is a secreted glycosidase. In another embodiment the glycosidase is active in the extracellular environment.

In one embodiment, the glycosidase has a substrate that is an N-linked glycan. In one embodiment, the glycosidase deglycosylates N-linked glycans. In one embodiment, the glycosidase is an N-glycanase.

In one embodiment, the glycosidase has a substrate that is an O-linked glycan. In one embodiment, the glycosidase deglycosylates O-linked glycans. In one embodiment, the glycosidase is an O-glycanase.

In one embodiment, the glycosidase catalyzes the removal of one or more sugar moieties.

In one embodiment, the glycosidase catalyzes the removal of an entire glycan chain.

As used herein, “a glycan” may refer to an entire glycan structure, or fragments of said structure, such as individual sugars (mono-, di-, poly-saccharides).

An exemplary glycosidase is peptide-N(4)-(N-acetyl-beta-glucosaminyl) asparagine amidase (PNGase), such as human PNGase and modified versions thereof.

In one embodiment, the glycosidase is PNGase.

In one embodiment, the glycosidase is human PNGase.

Exemplary human PNGase [Uniprot (Q96IV0-1)] (SEQ ID NO: 66):
AAAALGSSSGSASPAVAELCQNTPETFLEASKLLLTYADNILRNPNDEKYRSIRIGNT
AFSTRLLPVRGAVECLFEMGFEEGETHLIFPKKASVEQLQKIRDLIAIERSSRLDGSN
KSHKVKSSQQPAASTQLPTTPSSNPSGLNQHTRNRQGQSSDPPSASTVAADSAILE
VLQSNIQHVLVYENPALQEKALACIPVQELKRKSQEKLSRARKLDKGINISDEDFLLL
ELLHWFKEEFFHWVNNVLCSKCGGQTRSRDRSLLPSDDELKWGAKEVEDHYCDA
CQFSNRFPRYNNPEKLLETRCGRCGEWANCFTLCCRAVGFEARYVWDYTDHVWT
EVYSPSQQRWLHCDACEDVCDKPLLYEIGWGKKLSYVIAFSKDEVVDVTWRYSCK
HEEVIARRTKVKEALLRDTINGLNKQRQLFLSENRRKELLQRIIVELVEFISPKTPKPG
ELGGRISGSVAWRVARGEMGLQRKETLFIPCENEKISKQLHLCYNIVKDRYVRVSN
NNQTISGWENGVWKMESIFRKVETDWHMVYLARKEGSSFAYISWKFECGSVGLKV
DSISIRTSSQTFQTGTVEWKLRSDTAQVELTGDNSLHSYADFSGATEVILEAELSRG
DGDVAWQHTQLFRQSLNDHEENCLEIIIKFSDL

In one embodiment, the glycosidase is a variant of human PNGase. In one embodiment the glycosidase comprises or consists of a sequence with at least 70% sequence identity, such as at least 75%, at least 80%, at least 85%, at least 90%, at least 95%, at least 99%, or 100% sequence identity to SEQ ID NO: 66, or a fragment thereof.

In one embodiment, the PNGase variant is a truncated PNGase.

In one embodiment, the PNGase lacks a PUB domain. In another embodiment the PNGase lacks a PAW domain. In a further embodiment, the PNGase lacks both a PUB and a PAW domain.

Exemplary PUB truncated human PNGase (SEQ ID NO: 67):
KASVEQLQKIRDLIAIERSSRLDGSNKSHKVKSSQQPAASTQLPTTPSSNPSGLNQH
TRNRQGQSSDPPSASTVAADSAILEVLQSNIQHVLVYENPALQEKALACIPVQELKR
KSQEKLSRARKLDKGINISDEDFLLLELLHWFKEEFFHWVNNVLCSKCGGQTRSRD
RSLLPSDDELKWGAKEVEDHYCDACQFSNRFPRYNNPEKLLETRCGRCGEWANC
FTLCCRAVGFEARYVWDYTDHVWTEVYSPSQQRWLHCDACEDVCDKPLLYEIGW
GKKLSYVIAFSKDEVVDVTWRYSCKHEEVIARRTKVKEALLRDTINGLNKQRQLFLS
ENRRKELLQRIIVELVEFISPKTPKPGELGGRISGSVAWRVARGEMGLQRKETLFIPC
ENEKISKQLHLCYNIVKDRYVRVSNNNQTISGWENGVWKMESIFRKVETDWHMVYL
ARKEGSSFAYISWKFECGSVGLKVDSISIRTSSQTFQTGTVEWKLRSDTAQVELTG
DNSLHSYADFSGATEVILEAELSRGDGDVAWQHTQLFRQSLNDHEENCLEIIIKFSD
L
Exemplary PUB and PAW truncated human PNGase (SEQ ID NO: 68):
KASVEQLQKIRDLIAIERSSRLDGSNKSHKVKSSQQPAASTQLPTTPSSNPSGLNQH
TRNRQGQSSDPPSASTVAADSAILEVLQSNIQHVLVYENPALQEKALACIPVQELKR
KSQEKLSRARKLDKGINISDEDFLLLELLHWFKEEFFHWVNNVLCSKCGGQTRSRD
RSLLPSDDELKWGAKEVEDHYCDACQFSNRFPRYNNPEKLLETRCGRCGEWANC
FTLCCRAVGFEARYVWDYTDHVWTEVYSPSQQRWLHCDACEDVCDKPLLYEIGW
GKKLSYVIAFSKDEVVDVTWRYSCKHEEVIARRTKVKEALLRDTINGLNKQRQLFLS
ENRRKELLQRIIVELVEFISPKTPKPG
Exemplary PAW truncated human PNGase (SEQ ID NO: 69):
AAAALGSSSGSASPAVAELCQNTPETFLEASKLLLTYADNILRNPNDEKYRSIRIGNT
AFSTRLLPVRGAVECLFEMGFEEGETHLIFPKKASVEQLQKIRDLIAIERSSRLDGSN
KSHKVKSSQQPAASTQLPTTPSSNPSGLNQHTRNRQGQSSDPPSASTVAADSAILE
VLQSNIQHVLVYENPALQEKALACIPVQELKRKSQEKLSRARKLDKGINISDEDFLLL
ELLHWFKEEFFHWVNNVLCSKCGGQTRSRDRSLLPSDDELKWGAKEVEDHYCDA
CQFSNRFPRYNNPEKLLETRCGRCGEWANCFTLCCRAVGFEARYVWDYTDHVWT
EVYSPSQQRWLHCDACEDVCDKPLLYEIGWGKKLSYVIAFSKDEVVDVTWRYSCK
HEEVIARRTKVKEALLRDTINGLNKQRQLFLSENRRKELLQRIIVELVEFISPKTPKPG

In one embodiment, the glycosidase comprises or consists of:
(a) a sequence that has at least 70% sequence identity to SEQ ID NO: 66, or a fragment thereof;
(b) a sequence that has at least 70% sequence identity to SEQ ID NO: 67, or a fragment thereof;
(c) a sequence that has at least 70% sequence identity to SEQ ID NO: 68, or a fragment thereof; or
(d) a sequence that has at least 70% sequence identity to SEQ ID NO: 69, or a fragment thereof.

In one embodiment, the glycosidase comprises or consists of a sequence that has at least 70% sequence identity, such as at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 66-69, or a fragment thereof.

Glycosidases may comprise a signal peptide to facilitate their secretion. In one embodiment, the glycosidase comprises a signal peptide. The signal peptide may be cleaved from the glycosidase during its export from a cell.

In a preferred embodiment, the nucleotide sequence encoding a glycosidase further encodes a signal peptide operably linked to the glycosidase.

In one embodiment, the signal peptide is selected from the group consisting of: a CD8 signal peptide, an IgG variable region heavy chain signal peptide, an Ig kappa chain V-III region VG signal peptide, a GM-CFS/CSF signal peptide, and a CSFR2A signal peptide.

In one embodiment, the signal peptide is a CD8 signal peptide.

In one embodiment, the signal peptide is an IgG variable region heavy chain signal peptide.

An exemplary CD8 signal peptide [residues 1-12; Uniprot accession P01732] (SEQ ID NO: 71) is:

MALPVTALLLPLALLLHAARP

In one embodiment, the signal peptide comprises or consists of a sequence that has at least 70% sequence identity, such as at least 75%, 80%, 85%, 90%, 91%, 92%, 93%, 94%, 95%, 96%, 97%, 98%, 99%, or 100% sequence identity to SEQ ID NO: 71, or a fragment thereof. In one embodiment, the signal peptide comprises SEQ ID NO: 71. In one embodiment, the signal peptide consists of SEQ ID NO: 71.

An exemplary IgG variable region heavy chain signal peptide [residues 1-19; Uniprot accession P01768] (SEQ ID NO: 72) is:

MEFGLSWVFLVALLRGVQC

In one embodiment, the signal peptide comprises or consists of a sequence that has at least 70% sequence identity, such as at least 75%, 80%, 85%, 90%, 91%, 92%, 93%, 94%, 95%, 96%, 97%, 98%, 99%, or 100% sequence identity to SEQ ID NO: 72, or a fragment thereof. In one embodiment, the signal peptide comprises SEQ ID NO: 72. In one embodiment, the signal peptide consists of SEQ ID NO: 72.

An exemplary GM-CFS/CSF signal peptide [residues 1-17; Uniprot accession P04141] (SEQ ID NO: 73) is:

MWLQSLLLLGTVACSIS

In one embodiment, the signal peptide comprises or consists of a sequence that has at least 70% sequence identity, such as at least 75%, 80%, 85%, 90%, 91%, 92%, 93%, 94%, 95%, 96%, 97%, 98%, 99%, or 100% sequence identity to SEQ ID NO: 73, or a fragment thereof. In one embodiment, the signal peptide comprises SEQ ID NO: 73. In one embodiment, the signal peptide consists of SEQ ID NO: 73.

An exemplary Ig kappa chain V-III region VG signal peptide [residues 1-20; Uniprot accession P04433] (SEQ ID NO: 74) is:

MEAPAQLLFLLLLWLPDTTG

In one embodiment, the signal peptide comprises or consists of a sequence that has at least 70% sequence identity, such as at least 75%, 80%, 85%, 90%, 91%, 92%, 93%, 94%, 95%, 96%, 97%, 98%, 99%, or 100% sequence identity to SEQ ID NO: 74, or a fragment thereof. In one embodiment, the signal peptide comprises SEQ ID NO: 74. In one embodiment, the signal peptide consists of SEQ ID NO: 74.

An exemplary CSFR2A signal peptide [residues 1-22; NCBI ref. sequence NP_758452.1] (SEQ ID NO: 75) is:

MLLLVTSLLLCELPHPAFLLIP

In one embodiment, the signal peptide comprises or consists of a sequence that has at least 70% sequence identity, such as at least 75%, 80%, 85%, 90%, 91%, 92%, 93%, 94%, 95%, 96%, 97%, 98%, 99%, or 100% sequence identity to SEQ ID NO: 75, or a fragment thereof. In one embodiment, the signal peptide comprises SEQ ID NO: 75. In one embodiment, the signal peptide consists of SEQ ID NO: 75.

Further exemplary glycosidase sequences are set out below:

Exemplary human PNGase with CD8 signal peptide (SEQ ID NO: 76):
MALPVTALLLPLALLLHAARPAAAALGSSSGSASPAVAELCQNTPETFLEASKLLLTY
ADNILRNPNDEKYRSIRIGNTAFSTRLLPVRGAVECLFEMGFEEGETHLIFPKKASVE
QLQKIRDLIAIERSSRLDGSNKSHKVKSSQQPAASTQLPTTPSSNPSGLNQHTRNR
QGQSSDPPSASTVAADSAILEVLQSNIQHVLVYENPALQEKALACIPVQELKRKSQE
KLSRARKLDKGINISDEDFLLLELLHWFKEEFFHWVNNVLCSKCGGQTRSRDRSLLP
SDDELKWGAKEVEDHYCDACQFSNRFPRYNNPEKLLETRCGRCGEWANCFTLCC
RAVGFEARYVWDYTDHVWTEVYSPSQQRWLHCDACEDVCDKPLLYEIGWGKKLS
YVIAFSKDEVVDVTWRYSCKHEEVIARRTKVKEALLRDTINGLNKQRQLFLSENRRK
ELLQRIIVELVEFISPKTPKPGELGGRISGSVAWRVARGEMGLQRKETLFIPCENEKI
SKQLHLCYNIVKDRYVRVSNNNQTISGWENGVWKMESIFRKVETDWHMVYLARKE
GSSFAYISWKFECGSVGLKVDSISIRTSSQTFQTGTVEWKLRSDTAQVELTGDNSL
HSYADFSGATEVILEAELSRGDGDVAWQHTQLFRQSLNDHEENCLEIIIKFSDL
Exemplary human PNGase with IgG variable region heavy chain signal
peptide (SEQ ID NO: 77):
MEFGLSWVFLVALLRGVQCAAAALGSSSGSASPAVAELCQNTPETFLEASKLLLTY
ADNILRNPNDEKYRSIRIGNTAFSTRLLPVRGAVECLFEMGFEEGETHLIFPKKASVE
QLQKIRDLIAIERSSRLDGSNKSHKVKSSQQPAASTQLPTTPSSNPSGLNQHTRNR
QGQSSDPPSASTVAADSAILEVLQSNIQHVLVYENPALQEKALACIPVQELKRKSQE
KLSRARKLDKGINISDEDFLLLELLHWFKEEFFHWVNNVLCSKCGGQTRSRDRSLLP
SDDELKWGAKEVEDHYCDACQFSNRFPRYNNPEKLLETRCGRCGEWANCFTLCC
RAVGFEARYVWDYTDHVWTEVYSPSQQRWLHCDACEDVCDKPLLYEIGWGKKLS
YVIAFSKDEVVDVTWRYSCKHEEVIARRTKVKEALLRDTINGLNKQRQLFLSENRRK
ELLQRIIVELVEFISPKTPKPGELGGRISGSVAWRVARGEMGLQRKETLFIPCENEKI
SKQLHLCYNIVKDRYVRVSNNNQTISGWENGVWKMESIFRKVETDWHMVYLARKE
GSSFAYISWKFECGSVGLKVDSISIRTSSQTFQTGTVEWKLRSDTAQVELTGDNSL
HSYADFSGATEVILEAELSRGDGDVAWQHTQLFRQSLNDHEENCLEIIIKFSDL
Exemplary PUB truncated human PNGase with IgG variable region heavy
chain signal peptide (SEQ ID NO: 78):
MEFGLSWVFLVALLRGVQCKASVEQLQKIRDLIAIERSSRLDGSNKSHKVKSSQQP
AASTQLPTTPSSNPSGLNQHTRNRQGQSSDPPSASTVAADSAILEVLQSNIQHVLV
YENPALQEKALACIPVQELKRKSQEKLSRARKLDKGINISDEDFLLLELLHWFKEEFF
HWVNNVLCSKCGGQTRSRDRSLLPSDDELKWGAKEVEDHYCDACQFSNRFPRYN
NPEKLLETRCGRCGEWANCFTLCCRAVGFEARYVWDYTDHVWTEVYSPSQQRWL
HCDACEDVCDKPLLYEIGWGKKLSYVIAFSKDEVVDVTWRYSCKHEEVIARRTKVK
EALLRDTINGLNKQRQLFLSENRRKELLQRIIVELVEFISPKTPKPGELGGRISGSVA
WRVARGEMGLQRKETLFIPCENEKISKQLHLCYNIVKDRYVRVSNNNQTISGWENG
VWKMESIFRKVETDWHMVYLARKEGSSFAYISWKFECGSVGLKVDSISIRTSSQTF
QTGTVEWKLRSDTAQVELTGDNSLHSYADFSGATEVILEAELSRGDGDVAWQHTQ
LFRQSLNDHEENCLEIIIKFSDL
Exemplary PUB and PAW truncated human PNGase with IgG variable region
heavy chain signal peptide (SEQ ID NO: 79):
MEFGLSWVFLVALLRGVQCKASVEQLQKIRDLIAIERSSRLDGSNKSHKVKSSQQP
AASTQLPTTPSSNPSGLNQHTRNRQGQSSDPPSASTVAADSAILEVLQSNIQHVLV
YENPALQEKALACIPVQELKRKSQEKLSRARKLDKGINISDEDFLLLELLHWFKEEFF
HWVNNVLCSKCGGQTRSRDRSLLPSDDELKWGAKEVEDHYCDACQFSNRFPRYN
NPEKLLETRCGRCGEWANCFTLCCRAVGFEARYVWDYTDHVWTEVYSPSQQRWL
HCDACEDVCDKPLLYEIGWGKKLSYVIAFSKDEVVDVTWRYSCKHEEVIARRTKVK
EALLRDTINGLNKQRQLFLSENRRKELLQRIIVELVEFISPKTPKPG

In one embodiment, the glycosidase comprises or consists of a sequence that has at least 70% sequence identity, such as at least 75%, 80%, 85%, 90%, 91%, 92%, 93%, 94%, 95%, 96%, 97%, 98%, 99%, or 100% sequence identity to any one of SEQ ID NOs: 76-79 or a fragment thereof.

### Expression Control Sequences

The polynucleotide of the invention may comprise one or more expression control sequence. Suitably, the nucleic acid sequence encoding the CAR and/or any other polynucleotides of interest are operably linked to one or more expression control sequence. As used herein, the term “operably linked” means that parts (e.g. the nucleic acid sequence encoding the CAR and the one or more expression control sequence) are linked together in a manner which enables both to carry out their function substantially unhindered.

As used herein an “expression control sequence” may refer to a nucleotide sequence which controls expression of a transgene, e.g. to facilitate and/or increase expression.

The expression control sequence and the transgene may be in any suitable arrangement in the polynucleotide, providing that the expression control sequence is operably linked to the transgene (e.g. nucleic acid sequence encoding the CAR or any other nucleotide sequence of interest).

### Promoters

In some embodiments, the expression control sequence is a promoter.

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the nucleotide of interest (e.g. CAR) in a particular cell type (e.g. a tissue-specific promoter). The promoter may show inducible expression in response to presence of another factor, for example a factor present in a host cell. In any event, where the vector is administered for therapy, it is preferred that the promoter should be functional in the target cell background.

In some embodiments, the polynucleotide further comprises a promoter operably linked to the nucleic acid sequence encoding the CAR, scFv, or other nucleotide of interest.

In some embodiments, the promoter is a constitutive promoter.

In one embodiment, the nucleotide sequence encoding the CAR is operably linked to one or more promoter(s).

In one embodiment, the nucleotide sequences encoding the CAR and any other polynucleotide of interest are operably linked to one or more promoter(s).

In one embodiment, the nucleotide sequences encoding the CAR and any other polynucleotide of interest are operably linked to the same promoter.

The nucleotide sequences encoding the CAR and any other polynucleotide of interest may share a promoter such that their expression may be regulated by a single regulatory sequence.

In one embodiment, the nucleotide sequences encoding the CAR and polynucleotide of interest are independently operably linked to one or more promoter(s).

The nucleotide sequences encoding the polynucleotide of interest and the CAR may each be operably linked to a separate promoter such that their expression may be independently regulated by independent regulatory sequences. In one embodiment, the nucleotide sequences encoding the polynucleotide of interest and the CAR are operably linked to separate promoter(s).

In one embodiment, the polynucleotide of interest and the CAR are encoded in opposing directions.

In one embodiment, the polynucleotide of interest and the CAR are encoded in opposing directions and are independently operably linked to separate promoters.

In one embodiment, the polynucleotide of interest and the CAR are encoded in the same direction.

In one embodiment, the promoter is selected from the group consisting of: a cytomegalovirus promoter (CMV), a human phosphoglycerate kinase promoter (PGK), an EF-1α promoter and an inducible NFAT promoter.

In one embodiment, the promoter is a cytomegalovirus (CMV) promoter.

In another embodiment, the promoter is a minimal cytomegalovirus (mCMV or minCMV) promoter (mCMV, see, for example, Amendola (2005) Nat Biotech 23:108-116).

In one embodiment, the promoter is human phosphoglycerate kinase (PGK) promoter.

In one embodiment, the promoter is an EF-1α promoter.

In one embodiment, the promoter is an inducible NFAT promoter. The inducible module may be composed of a synthetic NFAT response element usually comprising repetitions of the consensus NFAT binding site placed upstream of a minimal promoter.

### Proteins

The term “protein” as used herein includes single-chain polypeptide molecules as well as multiple-polypeptide complexes where individual constituent polypeptides are linked by covalent or non-covalent means. The terms “polypeptide” and “peptide” as used herein refer to a polymer in which the monomers are amino acids and are joined together through peptide or disulfide bonds.

The proteins of the invention include any of the proteins disclosed herein with a methionine at the N-terminus.

### Polynucleotides

Polynucleotides of the invention may, for example, comprise DNA or RNA. They may be single-stranded or double-stranded. It will be understood by a skilled person that numerous different polynucleotides can encode the same polypeptide as a result of the degeneracy of the genetic code. In addition, it is to be understood that the skilled person may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to the skilled person. They may also be cloned by standard techniques.

Longer polynucleotides will generally be produced using recombinant means, for example using polymerase chain reaction (PCR) cloning techniques. This may involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

In one embodiment, the polynucleotide comprises a sequence encoding the CAR that comprises the sequence of any one of SEQ ID NOs: 16-18, 25-26, 46, or 51-54 or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the CAR that comprises the sequence of any one of SEQ ID NOs: 16-18, 25-26, 46, or 51-54.

In one embodiment, the polynucleotide comprises:
a) a sequence encoding the scFv that comprises the sequence of SEQ ID NO: 16 or 46; and/or
b) a sequence encoding the CAR that comprises the sequence of any one of SEQ ID NOs: 16-18, 25-26, or 51-54;
   or variants thereof, each having at least 75% sequence identity thereto.

In one embodiment, the polynucleotide comprises a sequence encoding the CAR that comprises the sequence of any one of SEQ ID NOs: 16-18, 25-26, 46, 51-54 or 85 or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the CAR that comprises the sequence of any one of SEQ ID NOs: 16-18, 25-26, 46, 51-54 or 85.

In one embodiment, the polynucleotide comprises:
a) a sequence encoding the scFv that comprises the sequence of SEQ ID NO: 16 or 46; and/or
b) a sequence encoding the CAR that comprises the sequence of any one of SEQ ID NOs: 16-18, 25-26, 51-54 or 85;
   or variants thereof, each having at least 75% sequence identity thereto.

In one embodiment, the polynucleotide comprises a sequence encoding the CAR that comprises the sequence of any one of SEQ ID NOs: 46 or 52-54 or a variant thereof having at least 75% sequence identity thereto, such as at least 80%, at least 85%, at least 90%, at least 91%, at least 92%, at least 93%, at least 94%, at least 95%, at least 96%, at least 97%, at least 98%, at least 99%, or 100% sequence identity thereto.

In one embodiment, the polynucleotide comprises a sequence encoding the CAR that comprises the sequence of any one of SEQ ID NOs: 46 or 52-54.

In one embodiment, the polynucleotide comprises:
a) a sequence encoding the scFv that comprises the sequence of SEQ ID NO: 46; and/or
b) a sequence encoding the CAR that comprises the sequence of any one of SEQ ID NOs: 52-54;
   or variants thereof, each having at least 75% sequence identity thereto.

### Vectors

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid or facilitating the expression of the protein encoded by a segment of nucleic acid. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid, mRNA or virus vectors and may include a promoter for the expression of a polynucleotide and optionally a regulator of the promoter.

Vectors comprising polynucleotides used in the invention may be introduced into cells using a variety of techniques known in the art, such as transfection, transformation and transduction. Several such techniques are known in the art, for example infection with recombinant viral vectors, such as retroviral, lentiviral (e.g. integration-defective lentiviral), adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14:556) and combinations thereof.

Transfection of cells with mRNA vectors can be achieved, for example, using nanoparticles, such as liposomes.

In some embodiments, the vector is comprised in a nanoparticle. In some embodiments, the nanoparticle is a polymeric nanoparticle, inorganic nanoparticle or lipid nanoparticle. In some embodiments, the nanoparticle is a liposome.

The nanoparticle may be targeted to a specific cell type(s) using one or more ligand displayed on its surface.

In one embodiment, polynucleotide delivery is transposon mediated.

In one embodiment, the polynucleotide is an mRNA. The mRNA may be comprised in a nanoparticle.

### Viral Vectors

In preferred embodiments, the vector is a viral vector. The viral vector may be in the form of a viral vector particle.

The viral vector may be, for example, a retroviral, lentiviral, adeno-associated viral (AAV) or adenoviral vector.

In some embodiments, the vector is a lentiviral vector. In some embodiments, the vector is an AAV vector.

### Retroviral and Lentiviral Vectors

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses. A review of these retroviruses is presented in Coffin et al. (1997) Retroviruses, Cold Spring Harbour Laboratory Press, 758-63.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective.

Lentivirus vectors are part of the larger group of retroviral vectors. A detailed list of lentiviruses may be found in Coffin et al. (1997) Retroviruses, Cold Spring Harbour Laboratory Press, 758-63. Lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis et al. (1992) EMBO J. 11:3053-8; Lewis et al. (1994) J. Virol. 68:510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site) or by modifying or deleting essential att sequences from the vector LTR, or by a combination of the above.

### Cells

The invention provides a cell comprising the CAR, scFv, polynucleotide, or the vector of the invention.

In one embodiment the cell is a mammalian cell. In another embodiment, the cell is a human cell.

In one embodiment, the cell is a cell from a subject. In one embodiment, the subject is a human subject.

In one embodiment, the cell is a T cell.

In one embodiment, the cell is a natural killer (NK) cell.

In one embodiment, the cell is a hematopoietic stem cell (HSC).

In one embodiment, the cell is a hematopoietic stem and/or progenitor cell (HSPC).

In one embodiment, the cell is a tumor-infiltrating lymphocyte (TIL).

In one embodiment, the cell is an invariant-NK T cell, a cytokine-induced killer cell (CIK) or a macrophage.

TILs are T cells that can be isolated from a tumor. TILs are enriched in natural T cells that recognize the tumor antigens. Isolated TILs can be expanded and modified, such as transduced with a polynucleotide or vector according to the invention, ex vivo and re-introduced to a tumor or subject.

The invention also contemplates a cell expressing a CAR of the invention, which has been engineered to disrupt one or more endogenous MHC genes. Disruption of an endogenous MHC gene can reduce or prevent expression of MHC on the engineered cell surface. Accordingly, such an engineered cell with reduced or no MHC expression will have limited or no capacity to present antigens on its cell surface. Such a cell is particularly advantageous for adoptive cell transfer since the cell will be non-alloreactive, e.g., the cell will not present antigens which could be recognized by the immune system of a subject receiving the adoptively transferred cell. As a result, the transferred cell will not be recognized as ‘non-self’ and an adverse immune reaction to the cell can be avoided. Such a cell is termed a ‘universal cell’ since it is suitable for adoptive transfer to a variety of different hosts regardless of HLA type.

In some embodiments, the cell is a non-alloreactive universal T-cell.

### Variants, Derivatives, Analogues, Homologues, and Fragments

In addition to the specific proteins and polynucleotides mentioned herein, the invention also encompasses the use of variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring protein.

The term “derivative” as used herein in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”.

A homologous sequence may include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

A homologous sequence may include a nucleotide sequence which may be at least 50%, 55%, 65%, 75%, 85% or 90% identical, preferably at least 95% or 97% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs disclosed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12:387). Examples of other software that can perform sequence comparisons include, but are not limited to, the BLAST package (see Ausubel et al. (1999) ibid-Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174:247-50; FEMS Microbiol. Lett. (1999) 177:187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. It is also possible to decrease expression by deliberately choosing codons for which the corresponding tRNAs are known to be rare in the particular cell type. Thus, an additional degree of translational control is available.

### Compositions

The polynucleotides, proteins (e.g., the CAR), vectors, and cells of the invention may be formulated for administration to subjects with a pharmaceutically-acceptable carrier, diluent or excipient. Suitable carriers and diluents include isotonic saline solutions, for example phosphate-buffered saline, and potentially contain human serum albumin.

Materials used to formulate a pharmaceutical composition should be non-toxic and should not interfere with the efficacy of the active ingredient. The precise nature of the carrier or other material may be determined by the skilled person according to the route of administration.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used. In some cases, serum albumin may be used in the composition.

For injection, the active ingredient may be in the form of an aqueous solution, which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Handling of the cell therapy products is preferably performed in compliance with FACT-JACIE International Standards for cellular therapy.

### Methods of Treatment

In one aspect, the invention provides the CAR, scFv, polynucleotide, vector, cell, composition or pharmaceutical composition of the invention for use in therapy.

In one aspect, the invention provides the CAR, scFv, polynucleotide, vector, cell, composition or pharmaceutical composition of the invention for use in the treatment of cancer.

All references herein to treatment include curative, palliative and prophylactic treatment. The treatment of mammals, particularly humans, is preferred. Both human and veterinary treatments are within the scope of the invention.

In some embodiments, the method of treatment provides the CAR, scFv, polynucleotide, vector, cell, composition or pharmaceutical composition of the invention to a tumor.

In one embodiment, the cancer is a solid tumor. In one embodiment, the cancer is a CDH17+ cancer.

In one embodiment:
a) the cancer is primary cancer, optionally a gastrointestinal cancer, a colorectal cancer, a pancreatic cancer and/or a stomach cancer;
b) the cancer is a secondary cancer, optionally a liver metastasis, optionally a liver metastasis of a colorectal cancer, and/or a liver metastasis of a pancreatic ductal adenocarcinoma (PDAC); and/or
c) the cancer is a neuroendocrine tumor.

In one embodiment, the cancer is primary cancer.

In one embodiment, the cancer is a gastrointestinal cancer, a colorectal cancer, a pancreatic cancer and/or a stomach cancer.

In one embodiment, the cancer is a gastrointestinal cancer.

In one embodiment, the cancer is a colorectal cancer.

In one embodiment, the cancer is a pancreatic cancer. In one embodiment, the cancer is a pancreatic ductal adenocarcinoma (PDAC). In one embodiment, the cancer is a pancreatic neuroendocrine tumor.

In one embodiment, the cancer is a stomach cancer.

In one embodiment, the cancer is a secondary cancer.

In one embodiment, the cancer is a liver metastasis.

In one embodiment, the cancer is a liver metastasis of a colorectal cancer.

In one embodiment, the cancer is a liver metastasis of a pancreatic ductal adenocarcinoma (PDAC).

In one embodiment, the cancer is a neuroendocrine tumor.

In one embodiment, the solid tumor is selected from the group consisting of: colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angio genesis, spinal axis tumor, brain stem glioma, pituitary adenoma, Kaposi's sarcoma, epidermoid cancer, squamous cell cancer, environmentally induced cancers, combinations of said cancers, and metastatic lesions of said cancers.

In one aspect, there is provided use of the polynucleotide, the vector, or the cell according to the invention, for improving CAR cell activity.

In one aspect, there is provided a method of producing a CAR cell, comprising introducing the CAR, scFv, polynucleotide, or the vector into a cell.

In one embodiment, the cell is a CAR-T cell.

In a further embodiment, there is provided the method or use, wherein the therapeutic activity of the CAR cell or CAR-T cell is improved.

In one embodiment, the therapeutic activity is target cell killing.

In one aspect, there is provided a method of treatment comprising producing a CAR cell according to the method of the invention, and administering the CAR cell to a subject in need thereof.

### Administration

In some embodiments, the CAR, scFv, polynucleotide, vector, cell, composition or pharmaceutical composition is administered to a subject locally.

Local administration may include administration to the tumor of interest.

In preferred embodiments, the CAR, scFv, polynucleotide, vector, cell, composition or pharmaceutical composition is administered to a tumor.

In some embodiments, the CAR, scFv, polynucleotide, vector, cell, composition or pharmaceutical composition is administered to a subject's liver. In some embodiments, the CAR, scFv, polynucleotide, vector, cell, composition or pharmaceutical composition is administered to a subject's pancreas.

In some embodiments, the CAR, scFv, polynucleotide, vector, cell, composition or pharmaceutical composition is administered to a subject systemically. In some embodiments, the CAR, scFv, polynucleotide, vector, cell, composition or pharmaceutical composition is administered to a subject intravenously.

The term “systemic delivery” or “systemic administration” as used herein means that the agent of the invention is administered into the circulatory system, for example to achieve broad distribution of the agent. In contrast, topical or local administration restricts the delivery of the agent to a localised area, e.g. a tumor.

In some embodiments, the CAR, scFv, polynucleotide, vector, cell, composition or pharmaceutical composition is administered in a nanoparticle that targets T cells in vivo.

### Dosage

The skilled person can readily determine an appropriate dose of an agent of the invention to administer to a subject. Typically, a physician will determine the actual dosage that will be most suitable for an individual patient, which will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

All publications mentioned in the above specification are herein incorporated by reference. Various modifications and variations of the disclosed polypeptides, polynucleotides, vectors, cells, compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

### EXAMPLES

### Material and Methods

### Expression Analysis in Liver Metastasis

Colorectal cancer (CRC) liver metastasis samples containing more than 70% of tumor cells were analyzed by mRNA sequencing. After obtaining the expression matrix in TPM (Transcripts Per Kilobase Million), values were converted in deciles and highly-expressed genes were defined as those belonging in the first three deciles (10, 9 and 8).

### Expression Analysis in Healthy Tissues

The approach described in Perna et al., Cancer Cell 2017 was applied for expression analysis in healthy tissues. The starting point was a global matrix including mRNA expression data of every gene in all available tissues. Means and standard deviations of all genes in each tissue were calculated to identify four expression classes: high, mid, low, and not detected. The class in which each candidate gene belonged in each tissue was determined and the criteria described in Perna et al. were applied to identify a safe target, namely “no high expression in any normal tissue except the tissue of origin” and “no mid expression across all healthy tissues”.

### Generation of CAR Constructs

Anti-CDH17 CARs were generated by including scFvs derived from either Lic3 or A4_4R mAbs (WO2017120557 and WO 2012054084). Both scFvs were synthesized by GeneArt (Thermo Fisher Scientific) and cloned into a CAR incorporating a CD28 trans-membrane and co-stimulatory domain, and a CD3z endodomain (Casucci, et al., Blood 2013). As CAR spacers, either a IgG1 hinge or an NGFR-derived sequence was incorporated as previously described (Casucci et al., Front Immunol 2018). All CARs were cloned into bidirectional lentiviral vectors under the direct control of the human phosphoglycerate kinase promoter (hPGK), whereas CD20 was cloned in antisense under the control of a minimal core promoter derived from the cytomegalovirus (minCMV or mCMV). Viral supernatants were produced in 293T packaging cells.

### Cells and Cell Culture

T cells were derived from peripheral blood of healthy donors after gradient centrifugation. All procedures were approved by the Institutional Review Board of IRCCS San Raffaele Scientific Institute and were compliant with all relevant ethical regulations. T cells were activated with CD3/CD28 beads (Gibco, 40203D) at a 3:1 ratio, transduced at day 2 and cultured in RPMI-1640 with interleukin (IL)-7 and IL-15 (5 ng/ml, Peprotech, 200-07, 200-15). At day 6, beads were removed and CAR T cells were expanded in complete medium until Day 21. Cell lines (LoVo and BxPC-3) were cultured in RPMI-1640 (Euroclone, ECB90062L). All media for tumor cells was supplemented with penicillin/streptomycin (100 UI/ml; Lonza, DE17-602E), glutamine (2 mM; Lonza, LOBE17-605E) and 10% FBS (fetal bovine serum, Carlo Erba, FA30WS1810500). All cells were routinely tested for mycoplasma contamination by PCR (Mycoplasmacheck, Eurofins Genomics) and proved negative.

In Vitro Co-Culture Assays with CAR T Cells

CAR T cells were co-cultured with target cells at different effector:target (E:T) ratios in RPMI-1640 fully supplemented in the absence of cytokines. After 4 days, surviving cells were counted using Flow-Count Fluorospheres (Beckman Coulter, 7547053) and analyzed by flow cytometry. T cells that were untransduced or transduced with an irrelevant CAR (19.28z) were used as a control. Elimination index was calculated as follows: 1−(number of residual target cells with experimental CAR T cells/number of residual target cells with control T cells).

### Flow Cytometry

Samples were washed with phosphate-buffered saline (PBS) containing 1% fetal bovine serum (FBS) and stained at 4° C. for 20 min. Prior to use, all antibodies were validated and titrated for the optimal on target/off target activity on human peripheral blood cells or tumor cell lines. Mouse anti-human fluorophore-conjugated antibodies specific for: CD3 allophycocyanin (APC)-Cy7 Cy7 (clone SK7, BioLegend, 344818), CD45 APC-Cy7 (clone HI30, BioLegend, 304014), CD45 PE-Cy7 (clone HI30, BioLegend, 304016), CD45 BV510 (clone 30-F11, BioLegend, 103137), CD45RA fluorescein isothiocyanate (FITC, clone HI100, BioLegend, 983002), CD62L APC (clone DREG-56, BioLegend, 304810), major histocompatibility complex II receptor (HLA-DR) APC-Cy7 (clone L243, BioLegend, 307618), programmed cell death protein 1 (PD-1) PE-Cy7 (clone EH12.2H7, BioLegend, 329918), and 4′,6-diamidino-2-phenylindole (DAPI) were used. CAR transduction efficiency was determined by staining with an anti-CD20 Pe (clone 2H7, BioLegend, 302306). Data were collected using FACS Canto (BD Biosciences) flow cytometer and analyzed using the FlowJo Software.

### Statistical Analysis

All data are presented as mean±s.e.m. Statistical analysis was performed on GraphPad Prism 8 software. Datasets were analyzed with Student's t-test, one-way or two-way ANOVA depending on the experimental design and as described in the figure legends. Differences with a P value<0.05 were considered statistically significant.

### Mouse Experiments

All experiments were approved by the Institutional Animal Care and Use Committee (IACUC) of IRCCS San Raffaele Scientific Institute and by the Italian Governmental Health Institute. Female or male 6 to 9-week-old NOD.Cg-Prkdescid II2rgtm1Wjl (NSG) mice (Charles River Laboratories) were kept in a specific-pathogen-free (SPF) facility within individually ventilated cages. Mice were injected intraperitoneally (i.p.) with luciferase (Luc+) LoVo CRC cells (0.5×10⁶) on day 0 and treated i.p. on day 11 with 6×10⁶ CAR T cells. Tumor growth was monitored by bioluminescence assay using the QUANTI-Luc detection reagent (InvivoGen, rep-qlc1) and expressed as relative light units (RLUs). Mice were euthanized at RLU≥5.5×10⁵ in control groups.

### Example 1: Identification of CDH17 as a Promising Target for CAR T-Cell Therapy of Multiple Solid Tumors

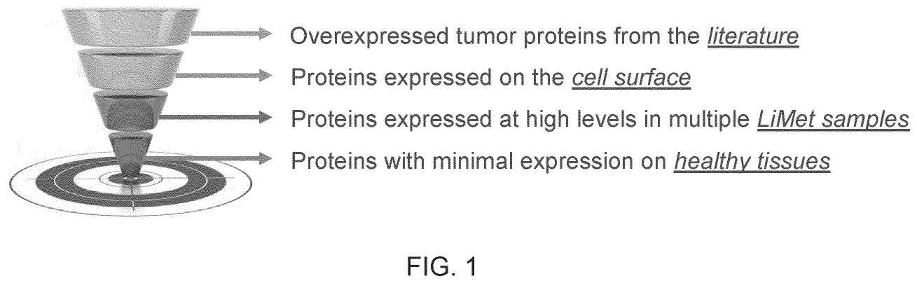

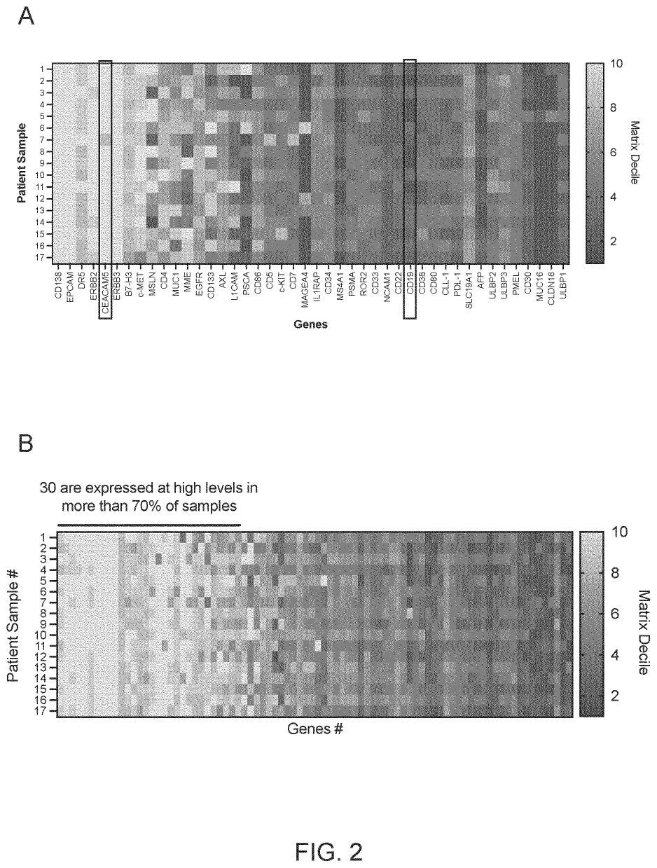

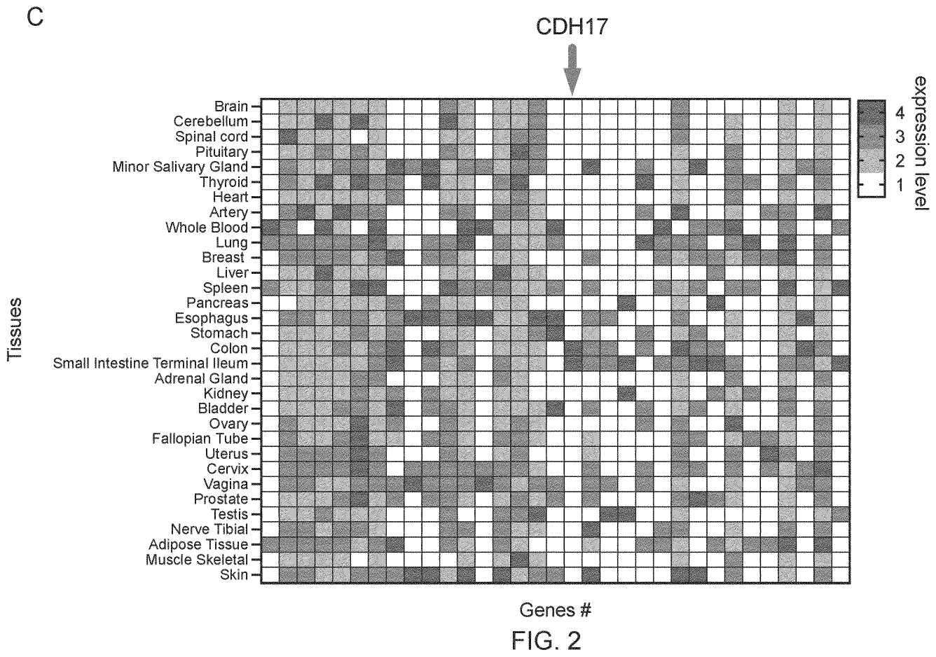

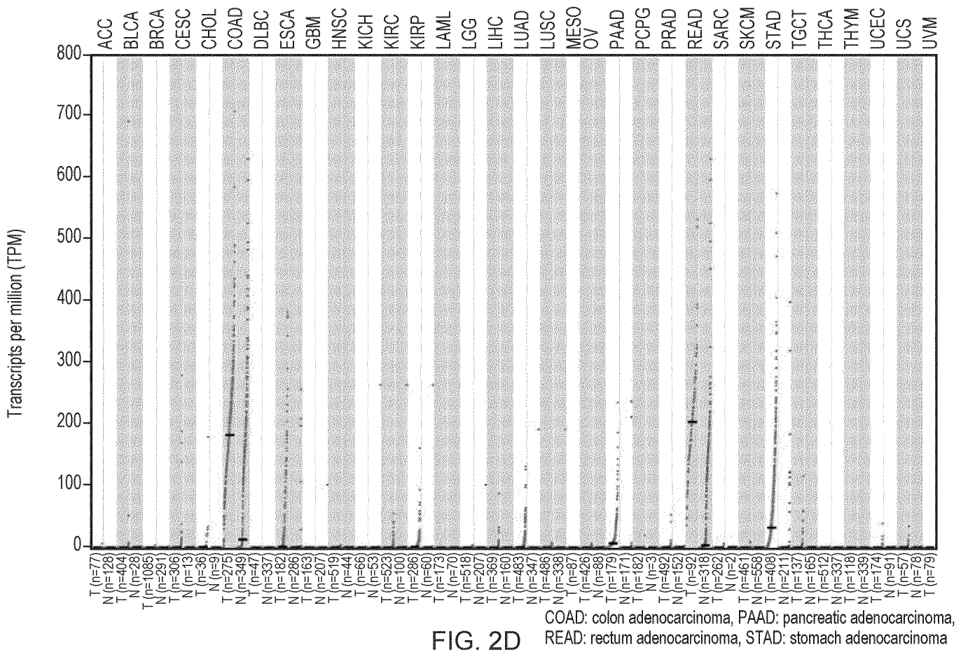

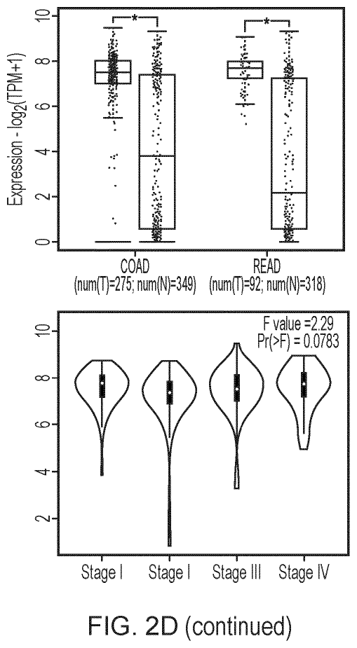

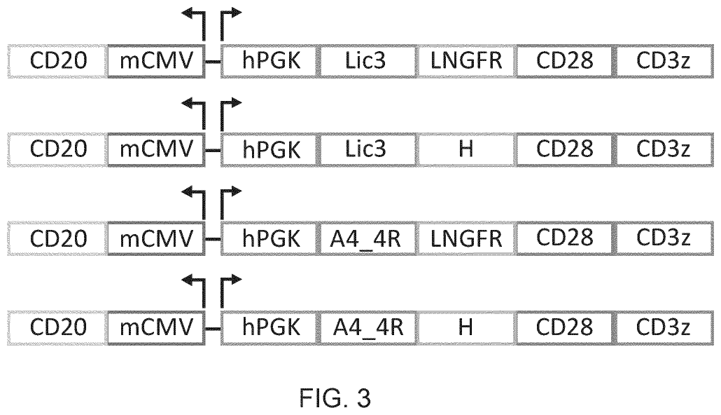

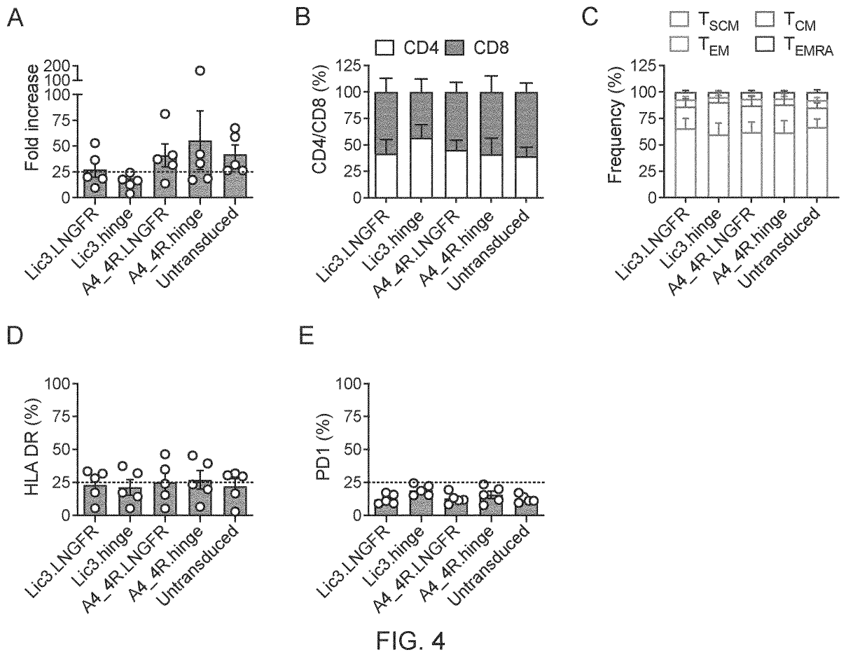

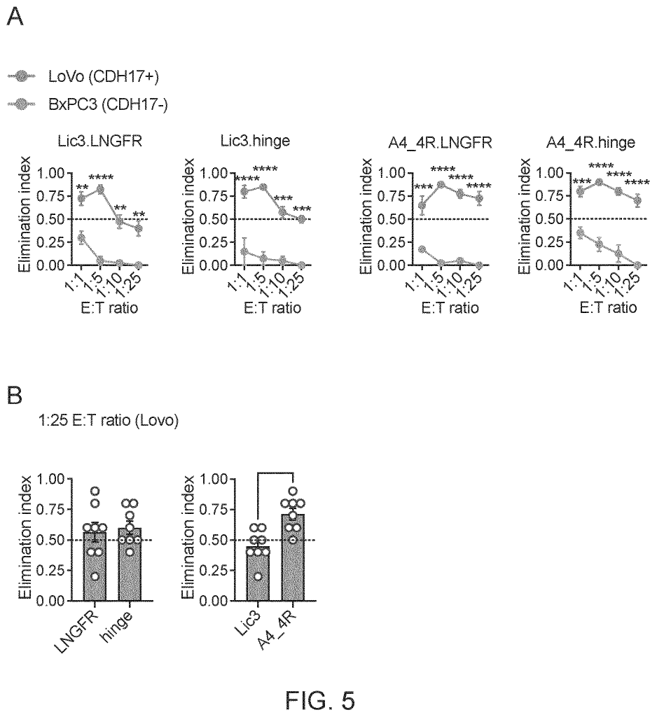

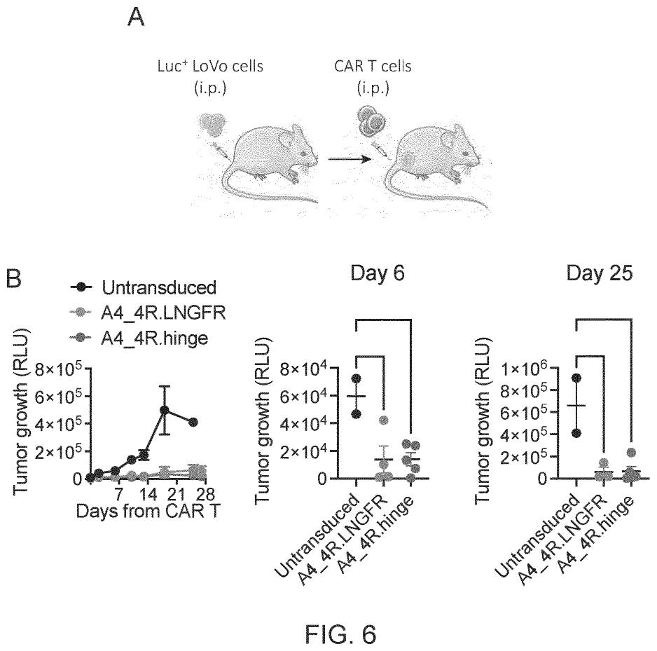

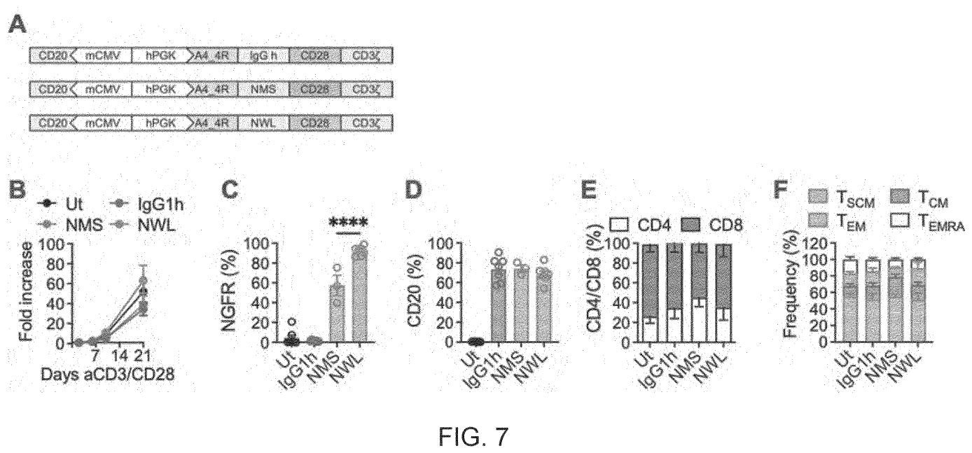

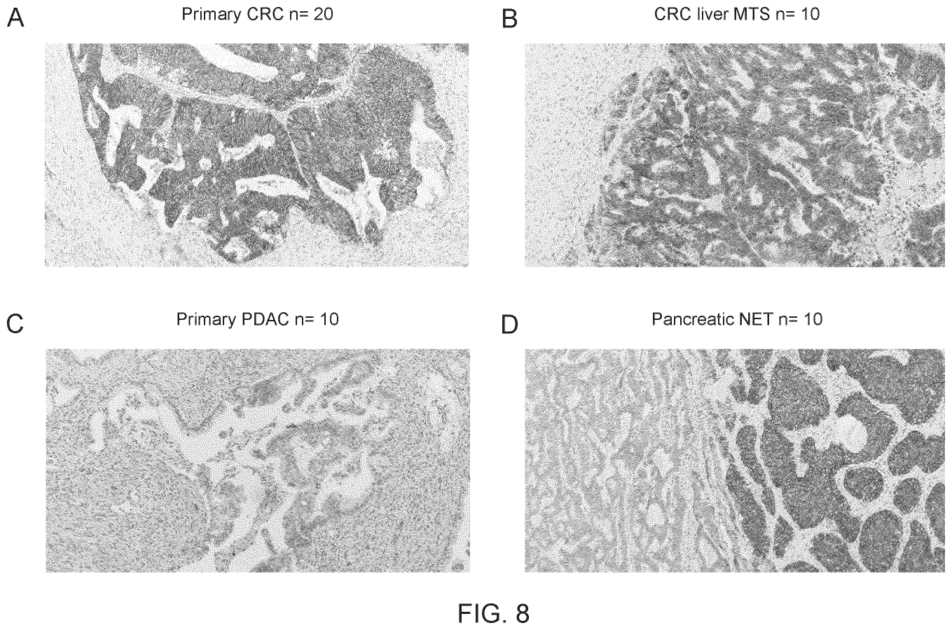

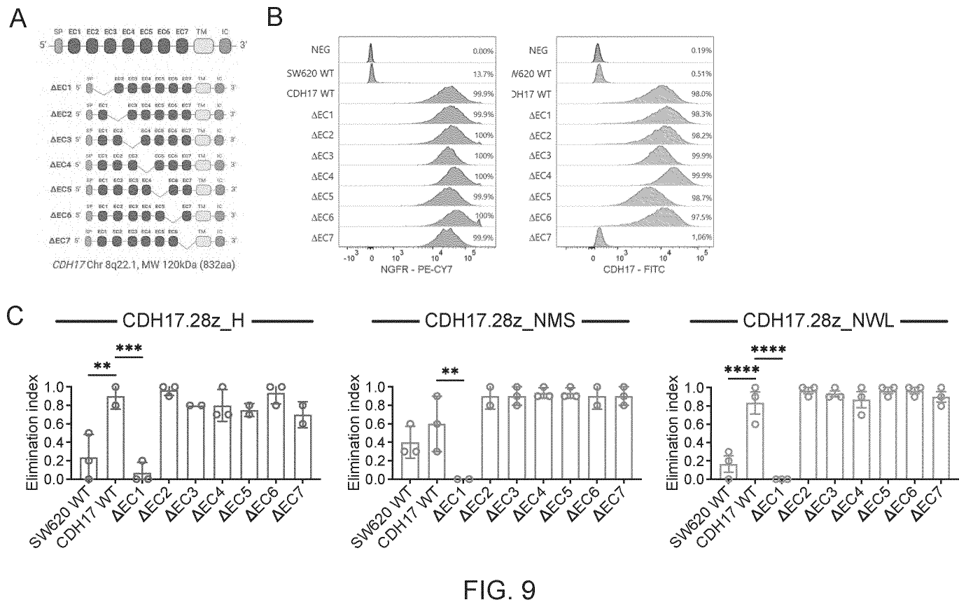

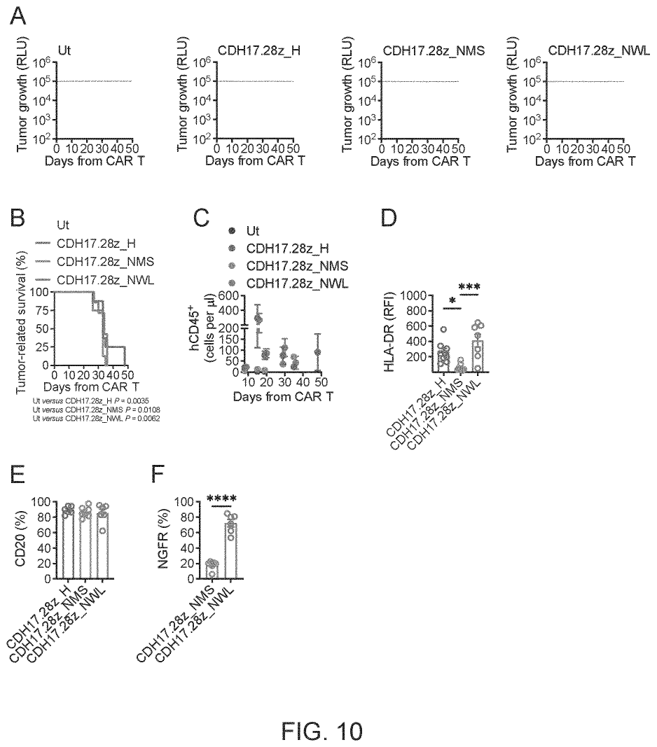

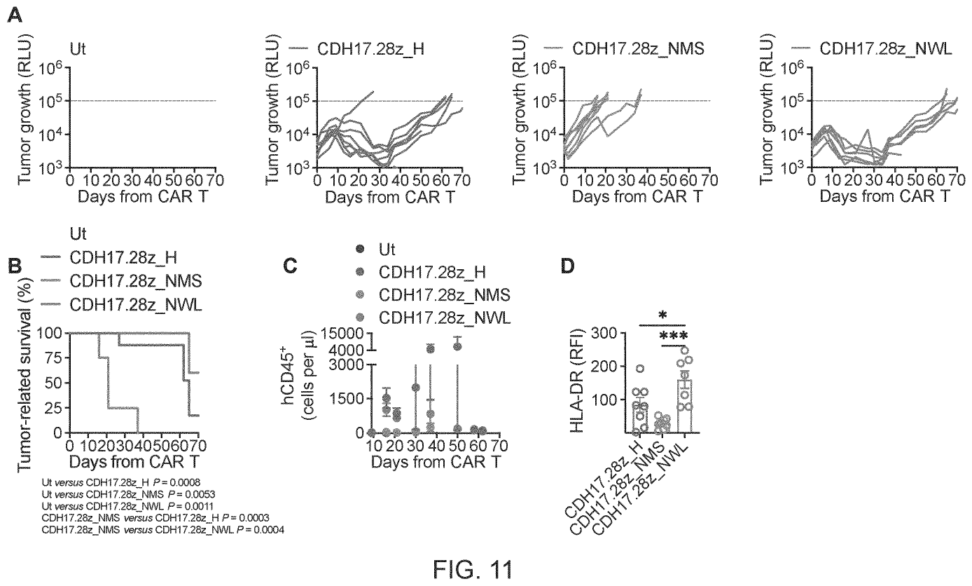

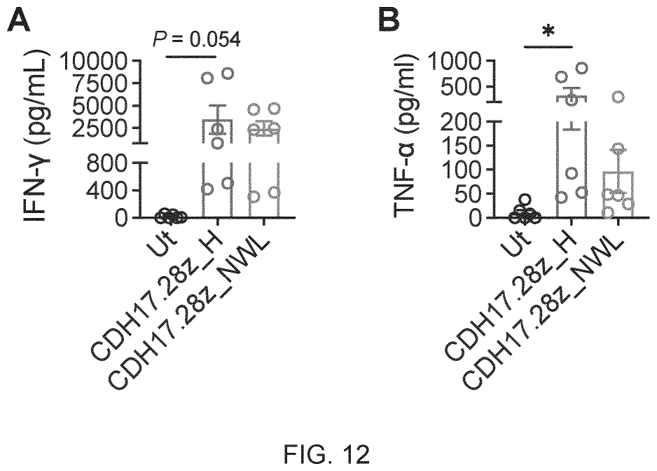

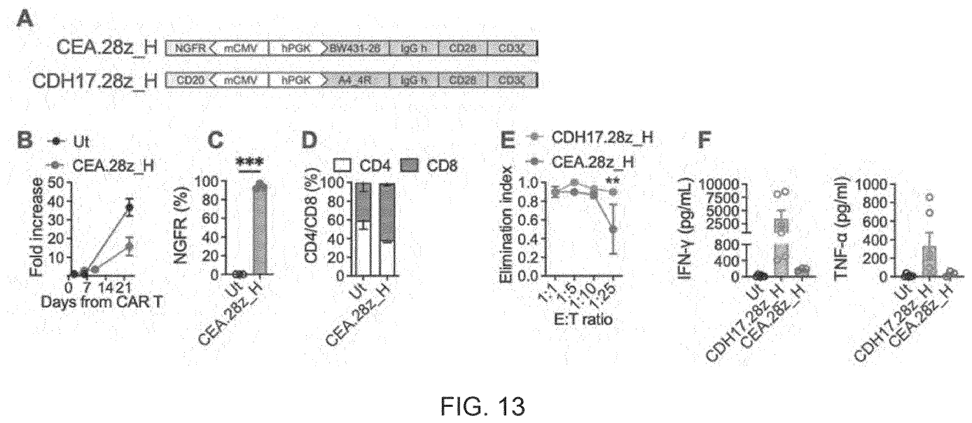

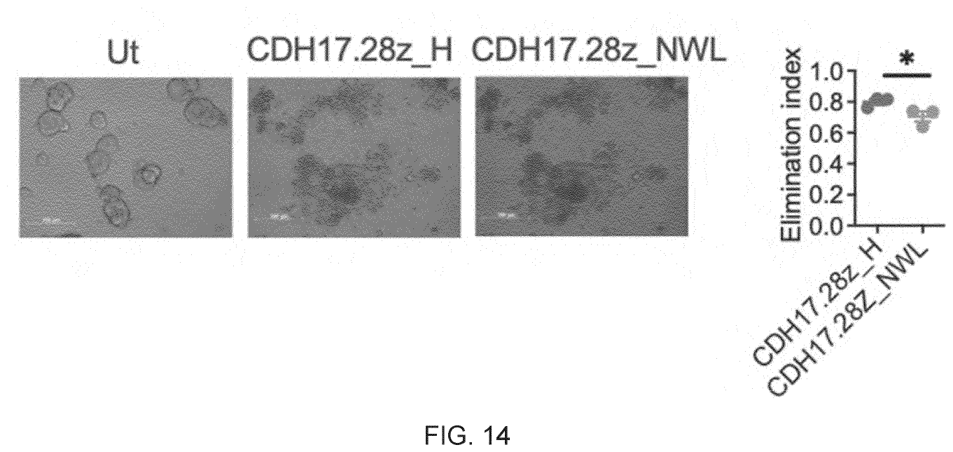

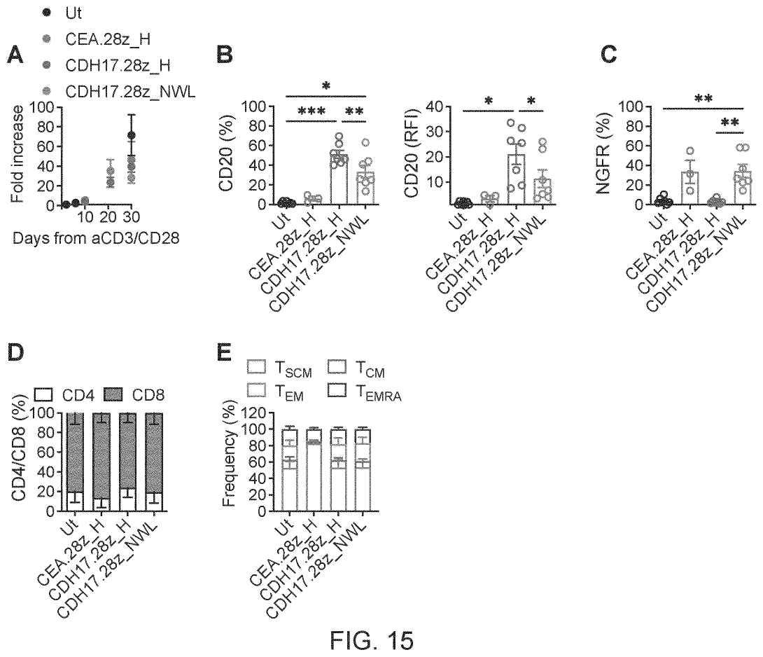

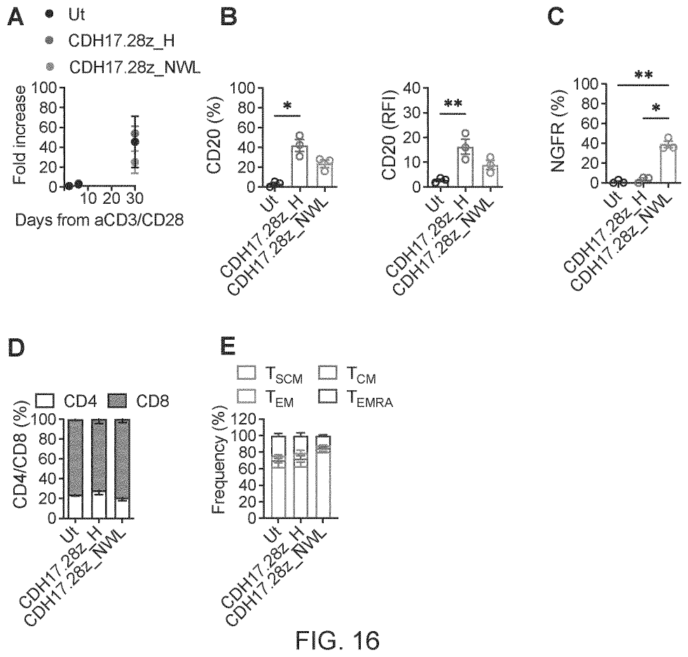

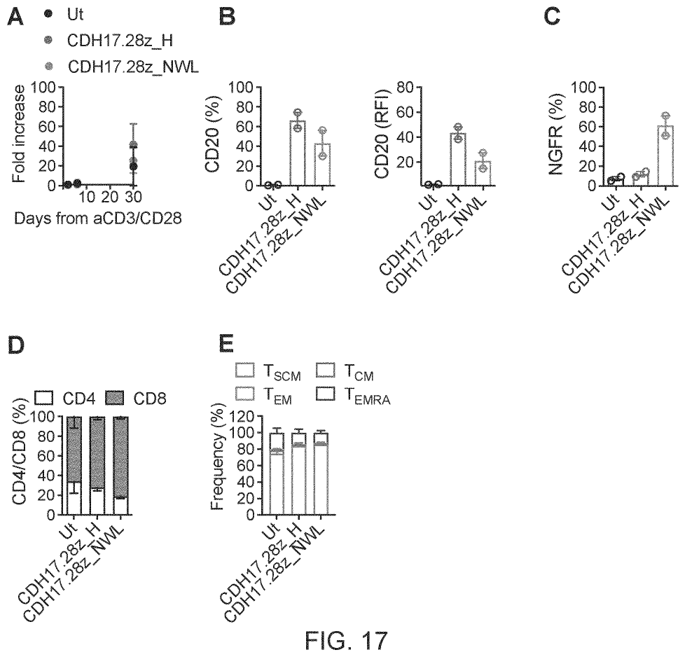

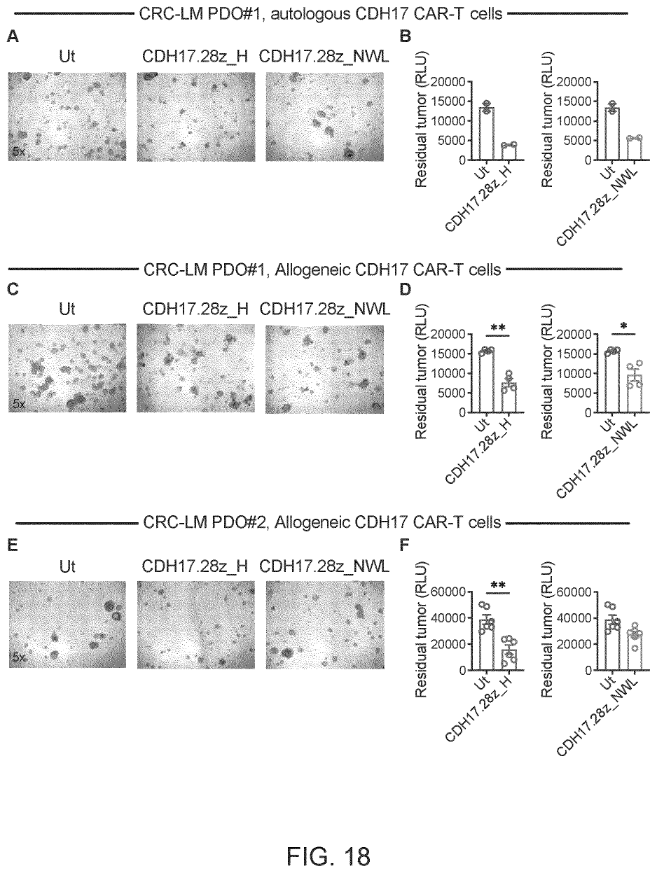

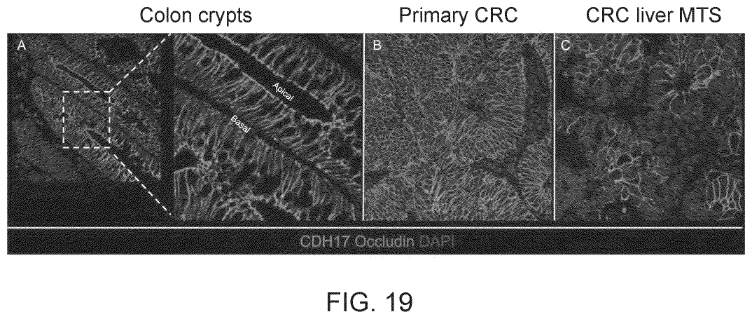

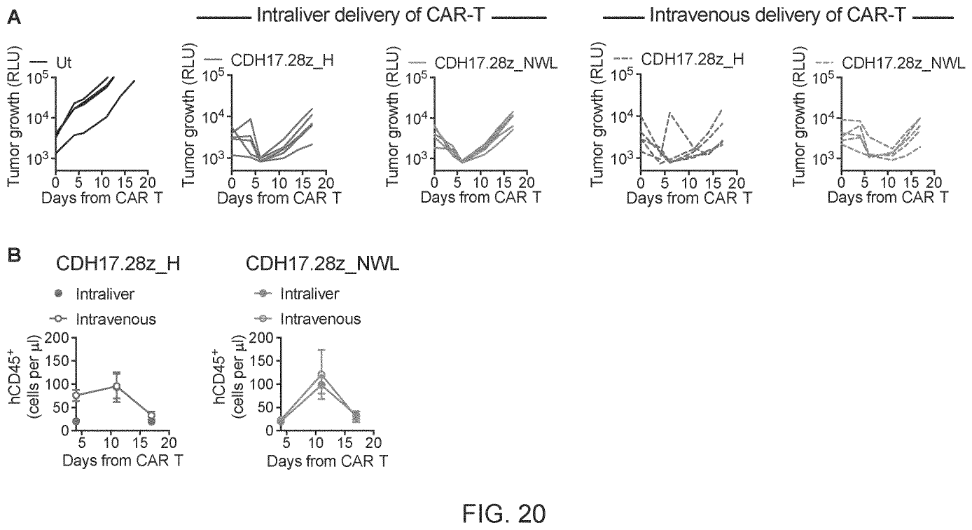

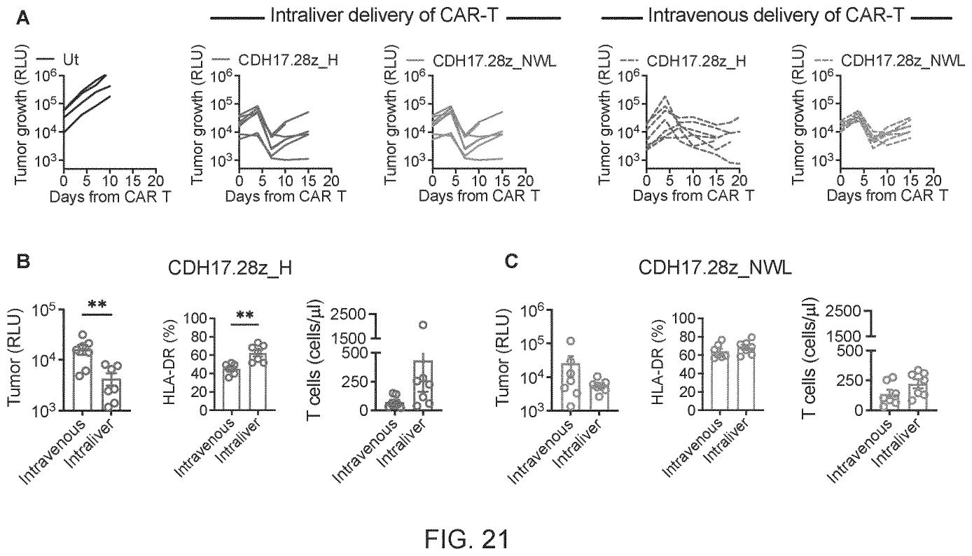

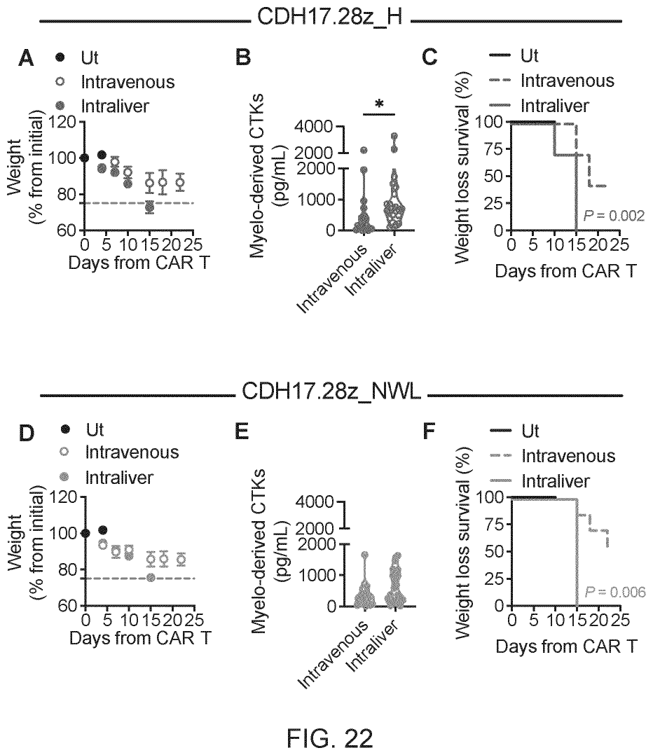

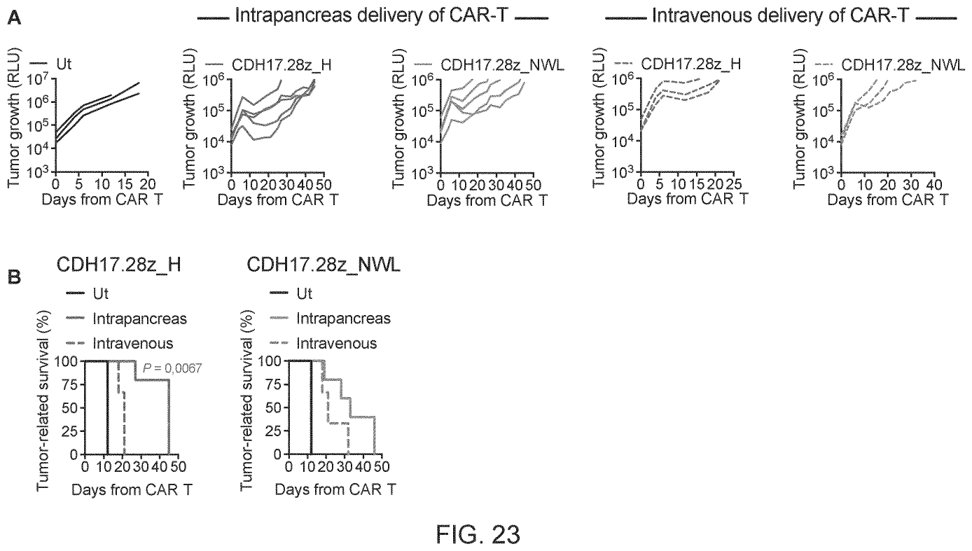

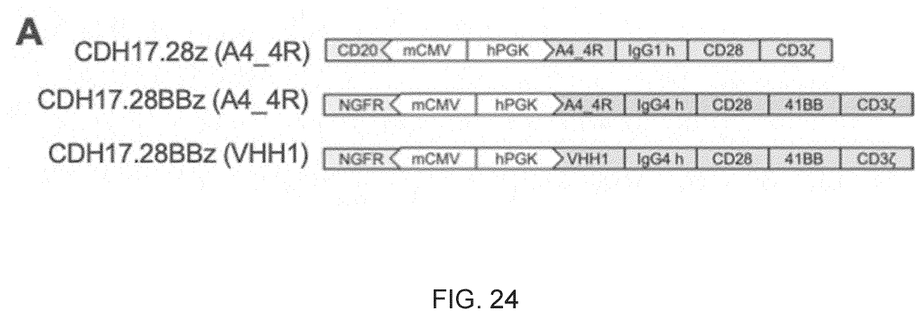

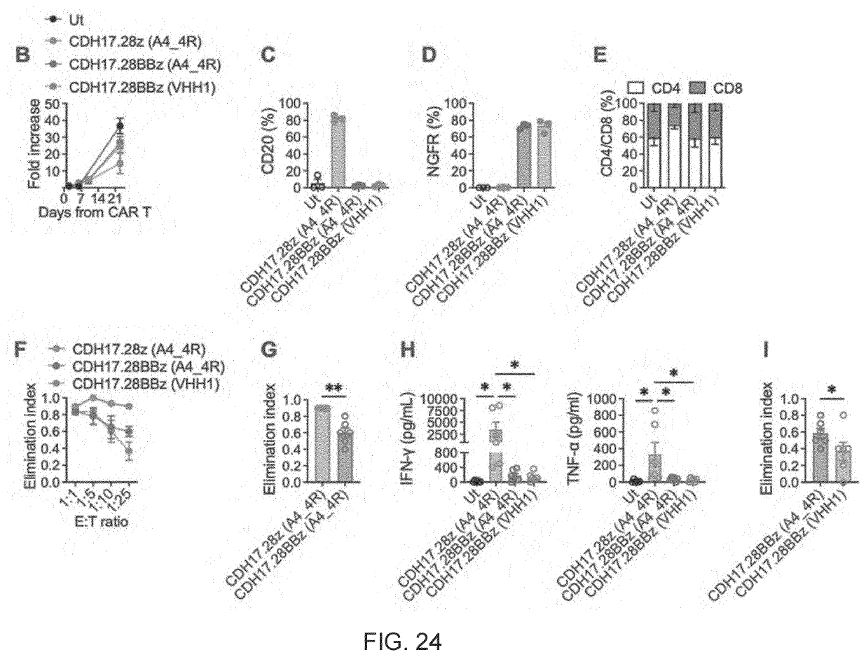

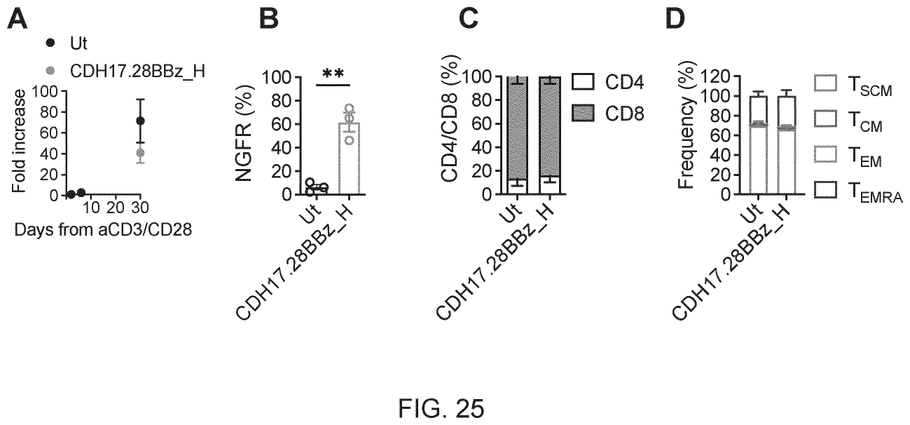

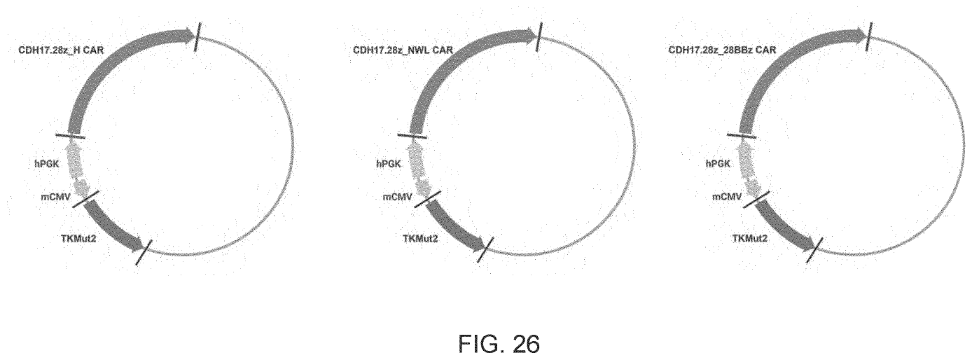

Identification of new targets for CRC liver metastasis was performed in a sequential fashion, as broadly illustrated in FIG. 1. First, articles were selected that included surface proteomic analyses of CRC and PDAC tumors and which comprised at least adjacent tissues as comparison. In addition, a list of CAR targets already in clinical development were included, to provide internal controls. From this analysis, a list of more than 200 genes was obtained and analyzed for their expression in LV MTS derived from the first cohort of CRC patients enrolled in the LiMeT protocol. This list was then reduced to 133 genes to meet the need for surface expression according to the HPA and UniProt. Analysis of the 47 known CAR antigens confirmed the appropriateness of our process, since no antigens were detected that were not expected to be found, such as CD19, while high expression of antigens, such as CEACAM5, was found, which are already reported to be highly expressed in LV MTS of different types of carcinomas (FIG. 2A). Interestingly, the analysis of the 86 new candidate antigens revealed 30 genes with high expression in more than 70% of patient samples (FIG. 2B). The expression of these 30 antigens in healthy tissues was checked by applying an approach described by Perna et al. Cancer Cell 2017, a matrix that reports the expression level of each candidate gene in each available tissue was generated (FIG. 2C). By applying the filtering criteria reported by Perna et al., CDH17 or Li cadherin, was identified as an antigen that satisfied all of the criteria, and one which is absent from all healthy tissues except the intestine.

CDH17 is a cell-to-cell adhesion glycoprotein that is overexpressed in several cancers of the gastrointestinal tract, including pancreas, colorectum, stomach, liver and esophagus, and in neuroendocrine tumors. Upon interaction with the @201 integrin, CDH17 promotes tumor cell proliferation, adhesion, and metastatic colonization of the liver.

Through use of the online database GEPIA, it was confirmed that CDH17 is overexpressed not only in CRC, but also in PDAC and stomach adenocarcinoma (FIG. 2D). Importantly, CDH17 is ten-fold over-expressed in tumor cells compared to the healthy counterparts, and such high expression is maintained across all CRC stages, including the metastatic stage.

### Example 2: Successful Generation of CDH17 CAR Constructs

New CAR constructs targeting CDH17 were devised and made. Single-chain-fragment variables (scFv) from two humanized antibody clones, Lic3 and A4_R4, were selected so as to limit the potential immunogenicity of the new CAR protein. For each scFv, two constructs were generated, which comprised different extracellular spacers: either an IgG1-derived hinge region or an LNGFR-derived spacer. The LNGFR-derived spacer includes the four TNFR cysteine-rich regions, where the fourth carries a deletion that abrogates NGF signalling, and is devoid of the serine/threonine-rich stalk (LNGFR mutated short or NMS, Casucci et al., Front Immunol 2018).

In all constructs, a CD28 as transmembrane and intracellular co-stimulatory domain were used as a starting point. The four constructs were cloned into bidirectional lentiviral vectors, together with the CD20 gene, which acted as both as a selection marker and a suicide gene in association with Rituximab (FIG. 3A).

### Example 3: CDH17 CAR T Cells Show Potent and Specific Recognition of CDH17+ Tumor Cells

Primary human T lymphocytes were transduced with the described LV vectors. To generate CDH17 CAR T cells, a protocol that can preserve T-cell fitness was applied, which is based on the use of anti-CD3/CD28 beads and IL-7/IL-15. No problems were encountered during manufacturing, with high expansion rates observed at the end of culture (FIG. 4A). CAR-T cell products obtained at the end of the process included a great representation of both CD4 and CD8, an extremely high fraction of stem memory T cells (T_{SCM}), a low activation profile and low expression of the PD-1 exhaustion marker (FIGS. 4B-E). When challenged in co-culture experiments, CDH17 CAR T cells showed potent elimination of CDH17-positive but not CDH17-negative tumor cells, indicating that all CARs are functional and specifically able to recognize the CDH17 antigen naturally expressed by malignant cells (FIG. 5A). Interestingly, no differences were observed between the two spacers, while the A4_4R scFv was superior to the Lic3 scFv (FIG. 5B).

The A4_4R.NMS CAR T cells and A4_4R.hinge CAR T cells were then tested in a xenograft mouse model of CRC with the LoVo cell line (FIG. 6A). In this model, LoVo cells were transduced to express a secreted luciferase, which allows the easy monitoring of tumor progression by serial peripheral blood analysis. While control untransduced T cells expectedly failed to control tumor progression, both A4_4R.NMS CAR T cells and A4_4R.hinge CAR T cells proved to be significantly effective (FIG. 6B).

Since the A4_4R scFv proved to be the best performing, in addition to the constructs that carry the NMS spacer (SEQ ID NO: 15) and the IgG1 hinge (SEQ ID NO: 14), we used this scFv to generate two additional CARs that include other spacer domains (SEQ ID Nos: 23 and 24). The first comprises the entire extracellular portion of the LNGFR molecule (LNGFR wild type long or NWL) and can be recognized by the clinical-grade anti-LNGFR mAb ME20.4. The second includes a mutated version of the IgG1 CH2CH3 spacer (mCH2CH3) that is unable to recognize the FcγRI.

### Example 4: Successful Generation of CDH17 CAR Constructs

Once selected the A4_4R scFv as the best-performing one, we devised and made an additional CAR construct and expanded the manufacturing testing with more donors. To this aim, alongside the IgG1 hinge and NWL-derived spacers, we generated an NGFR-derived mutated short (i.e. NMS) as described in Casucci et al. (2018) Front Immunol. The NMS-spaced CDH17 CAR was then cloned into the lentiviral backbone having the CD20 gene in antisense orientation (FIG. 7A). To generate CDH17 CAR-T cells, primary human T lymphocytes from healthy donors were stimulated with anti-CD3/CD28 beads, transduced with the described LV vectors and expanded with interleukin-7 (IL-7) and IL-15, according to a protocol that preserves T cell fitness. CAR-T cell products showed similar expansion kinetics and transduction efficiencies, despite a lower surface expression of NGFR by NMS-spaced CAR-T cells as compared to NWL-spaced CAR-T cells (FIGS. 7B-D). All T cell products included a great representation of both CD4 and CD8 (FIG. 7E), and an extremely high fraction of stem memory T cells (TSCM, FIG. 7F).

### Example 5: Expression of CDH17 in Human Primary Tumors

Immunohistochemistry analysis was performed to assess CDH17 protein expression in primary human tissue samples. Tissues from 20 patients with primary colorectal carcinoma (CRC; FIG. 8A) and from 10 patients with liver metastasis from CRC (FIG. 8B) were stained with a commercially available anti-CDH17 antibody (Abcam, Clone EPR 3996). CDH17 expression was found to be high and homogeneous in all tissue samples tested. This result aligns with previously reported findings showing high levels of CDH17 expression in tumor samples from CRC patients with advanced tumour and distant metastasis. Immunohistochemistry analysis of CDH17 expression resulted positive also toward tissues from primary pancreatic ductal adenocarcinoma (PDAC, FIG. 8C) and, more so, toward pancreatic neuroendocrine tumors (FIG. 8D).

### Example 6: Identification of the Extracellular Domain Containing the Epitope Recognized by A4_4R CARs

In order to better characterize and understand the interaction between A4_4R CAR T cells and CDH17 target antigen, we worked on the identification of the extracellular cadherin (EC) wherein the scFv binding site resides. Since CDH17 consists of seven EC domains that mediate calcium-dependent cell adhesion, we designed and generated seven distinct CDH17 mutants with individual EC domain deletion (FIG. 9A). The additional construct encoding for the wild-type (WT) form of CDH17 worked as a positive control. Full-length CDH17 and CDH17 mutant-encoding sequences were cloned in bidirectional lentiviral vectors carrying ΔNGFR selection marker in antisense orientation under mCMV promoter, and they were transduced into CDH17^{neg}SW620 colorectal cancer cells. Cells were efficiently transduced, as assessed by high levels of surface NGFR through flow cytometry, and expressed similar levels of CDH17 truncated mutants, as assessed by staining with the anti-CDH17 commercially available antibody (Santa Cruz anti-CDH17, clone H-1). As expected, since the anti-CDH17 antibody binds to an epitope located within EC7, the truncation of EC7 by the ΔEC7 mutant caused the loss of detectable signal (FIG. 9B). The transduced cells were then co-cultured with A4_4R CAR T cells carrying the three different extracellular spacers (i.e. CDH17.28z_H, CDH17.28z_NMS and CDH17.28z_NWL). A4_AR CAR T cells mediated an effective killing of all CDH17 mutants except for EC1 domain-deleted mutant (FIG. 9C). ΔEC1 was spared from CAR T cells killing, as well as WT SW620 cells that do not endogenously express CDH17 and worked as negative control, suggesting that the epitope recognized by A4_4R scFv lies within EC1.

### Example 7: In Vivo Evaluation of the Efficacy of CDH17.28z CAR T Cells with Different Extracellular Spacers in CRC and PDAC Xenograft Models

The efficacy of CDH17 CAR T cells carrying the three different extracellular spacers (i.e. CDH17.28z_H, CDH17.28z_NMS and CDH17.28z_NWL) was evaluated in vivo. We established tumor xenograft mouse models of colorectal cancer and pancreatic adenocarcinoma using LoVo and AsPC-1 cell lines, respectively. In both mouse models, tumor cells were subcutaneously injected and, in a setting of low-tumor burden, CDH17.28z CAR T cells were administered intravenously. In the CRC xenograft model, CDH17 CAR-T cells provided a significant survival benefit to mice as compared to control untransduced T cells (FIGS. 10A and B). However, CDH17.28z NMS showed no peripheral expansion and low T cell activation, while CDH17.28z_H and CDH17.28z_NWL CAR-T cells displayed high expansion peaks and activation rates (FIGS. 10C and D). Analysis of tumor-infiltrating lymphocytes retrieved at sacrifice showed that all three CDH17 CAR-T cells had high levels of the transduction marker gene CD20, suggesting that the integrating vector expressing the CAR was stably expressed in all conditions. However, CDH17.28z NMS CAR-T cells had lower levels of surface NGFR as compared to CDH17.28z_NWL CAR-T cells, suggesting lower surface expression of the CAR by CDH17.28z NMS CAR-T cells. Similar results were obtained in the PDAC xenograft model, whereby while all three CDH17 CAR-T cells provided a significant survival benefit to mice as compared to control untransduced T cells, CDH17.28z NMS CAR-T cells were the least effective in doing so (FIGS. 11A and B) and showed defects in the capability to expand in the periphery and activate as compared to CDH17.28z_H and CDH17.28z_NWL CAR-T cells (FIGS. 11C and D). Altogether, these results demonstrated the efficacy of CDH17.28z CAR T cells in recognizing and killing CDH17+ tumors in vivo and highlighted the lower efficacy of NMS-spaced CDH17 CAR-T cells.

### Example 8: Assessing Cytokine Production by CDH17.28z_H and CDH17.28z_NWL CAR-T Cells In Vitro

The production of IFNg and TNFa by CDH17.28z_H and CDH17.28z_NWL CAR-T cells was measured upon stimulation with the CRC LoVo cell line in vitro. Compared to control untransduced T cells, both CDH17 CAR-T cells produced considerable levels of cytokines (FIGS. 12A and B).

### Example 9: Efficacy of CEA and CDH17 CAR-T Cells In Vitro

CEA CAR-T cells are currently used in clinical trials for the treatment of liver metastasis originated from different solid tumors, including CRC. We therefore generated CEA CAR-T cells and compared the efficacy to CDH17 CAR-T cells. CEA CAR-T cells were generated by including an IgG hinge extracellular spacer and a CD28 transmembrane and costimulatory domain. The CEA CAR was cloned into bidirectional lentiviral vectors carrying NGFR as marker gene in antisense orientation (FIG. 13A). Primary human T lymphocytes from healthy donors were stimulated with anti-CD3/CD28 beads, transduced with the described LV vector and expanded with interleukin-7 (IL-7) and IL-15 (FIG. 13B). At the end of manufacturing, CEA CAR-T cells showed a high transduction efficiency and a great representation of both CD4 and CD8 (FIGS. 13C and D). Upon co-culture with LoVo tumors cells, both CEA and CDH17 CAR-T cells showed potent tumor recognition. Nonetheless, CDH17 CAR-T cells appeared more potent than CEA CAR-T cells, both in terms of tumor killing and of cytokine production (FIGS. 13E and F).

### Example 10: Efficacy of CDH17 CAR-T Cells with Primary Human Samples

To test the efficacy of CAR-T cells in more relevant settings, healthy donor-derived CDH17 CAR-T cells (see FIG. 7) were first tested against patient-derived organoids from CRC-LM. Patient-derived organoids are extremely relevant since they retain the histological complexity and genetic heterogeneity of parental tumors. CDH17.28z_H and CDH17.28z_NWL displayed potent recognition and killing of CRC-LM-derived organoids (FIG. 14).

Next, CDH17 CAR-T cells were manufactured from patients suffering from CRC-LM, PDAC-LM and PDAC (FIGS. 15, 16, 17). CDH17 CAR-T cells manufactured from CRC-LM patients displayed similar expansion kinetics and transduction efficiency as CEA CAR-T cells (FIGS. 15A-C), which are already employed in clinical trials. The distribution of CD4 and CD8 T cell compartments as well as T cell memory subsets were also comparable between the two CAR specificities (FIGS. 15D and E). Similar results were obtained with PDAC-LM and PDAC patients' T cell manufacturing, whereby CDH17 CAR-T cells showed high degrees of expansion and transduction (FIGS. 16 and 17, A-C) as well as a good representation of early memory T cell subsets, which are associated with improved antitumor activity in vivo (FIGS. 16 and 17, D and E).

Finally, patient-derived CDH17 CAR T cells from CRC-LM were co-cultured with autologous or allogeneic patient-derived organoids from CRC-LM. Compared to control untransduced T cells, CDH17 CAR-T cells displayed a significant potent recognition and killing of CRC-LM-derived organoids (FIG. 18).

### Example 11: Expression and Localization of CDH17 in Normal and Tumoral Human Colon

According to literature data and the expression analysis of healthy tissues (FIG. 2C), CDH17 is only expressed in the tissue of origin of colon carcinoma by epithelial cells of normal colon and small intestine. Reportedly, in normal cells the expression of CDH17 is localized at the cell-to-cell junctions between epithelial cells. This polarized expression is lost upon malignant transformation and tumor cells express the antigen all over cell surface. Murine CDH17 displays a similar expression pattern in normal colon and it has been demonstrated in animal models that the localization of the antigen at the cell-to-cell junctions masks the recognition by anti-CDH17 CAR-T cells (Feng et al. (2022) Nat Cancer), which encourages the safety of using CDH17-directed CAR-T cells. To address if CDH17 is localized at the lateral junctions between epithelial cells, we performed an immunofluorescence staining of human healthy colon tissues using CDH17 and Occludin, which is a marker for apical tight junctions in colonic epithelial cells. In normal colon, CDH17 was mainly expressed in the lateral side of epithelial cells but not at the basal side (FIG. 19A), which is the one accessible to CAR-T cells. On the contrary, tumor tissues showed a disruption of tissue architecture and CDH17 is expressed at high and continuous levels by tumoral epithelial cells (FIGS. 19B and C).

### Example 12: Validation of CDH17.28z Cells Administration Route in Xenograft Mouse Model of Colorectal Cancer

After having assessed the in vivo efficacy of CDH17.28z_H and NWL CAR-T cells, we moved to their optimization by validating first of all their administration route to potentially enhance their therapeutic effect. Locoregional delivery of CAR-T cells opposed to the conventional systemic administration has been explored as a means to overcome poor T cell trafficking and inefficient T cell penetration into solid tumors. To test locoregional delivery of CDH17.28z CAR-T cells, we used an immunodeficient NSG xenograft mouse model in which LoVo CRC cells are directly injected in the liver of mice and CDH17.28z_H CAR-T cells and CDH17.28z_NWL CAR-T cells were administered intraliver or intravenous. Both IgG1 hinge and NWL-spaced CAR-T cells proved an efficient suppression of tumor growth compared to control untransduced T cells with no significant differences between systemic and locoregional administration (FIG. 20A). In line with this, CDH17.28z_H and CDH17.28z_NWL CAR-T cells administered locoregionally or systemically expanded in a comparable manner, reaching a peak at day 11 post infusion (FIG. 20B). The locoregional versus systemic delivery of CDH17.28z CAR-T cells was also tested in a humanized model mouse model in which the CRC LoVo cell line was injected intraliver (FIG. 21). Briefly, in this model NSG mice transgenic for the expression of human IL-3, GM-CSF and CSF are reconstituted with human hematopoietic stem and progenitor cells to recreate a functional human immune system. Here, tumor growth and normal hematopoiesis coexist and this offers several advantages. First, the presence of cells and cytokines of human origin sustains human CAR-T cells dynamics in the long term and allow for more representative efficacy studies, mainly through the production of IL-15 by myeloid cells. Next, the model allows to recapitulate human CAR-T cells toxicities, such as the cytokine release syndrome (CRS), thanks to the presence of circulating monocytes which are primarily responsible for the systemic release of cytokines which causes the syndrome. In this model, no significant overall differences were observed in the capability of counteracting tumor growth by CDH17 CAR-T cells infused systemically or locoregionally (FIG. 21A). Nonetheless, the intraliver infusion of CDH17 CAR-T cells achieved a faster tumor debulking, T cell activation and T cell expansion (FIGS. 21B and C). However, possibly the stronger activation observed with the intraliver delivery of CDH17 CAR-T cells caused a harsh cytokine-release syndrome, which manifested as rapid weight loss (FIGS. 22A and D), higher levels of myelo-derived cytokines (FIGS. 22B and E) and eventually all mice treated intraliver succumbed to the toxicity (FIGS. 22C and F).

### Example 13: Validation of CDH17.28z CAR-T Cells Administration Route in Xenograft Mouse Model of Pancreatic Adenocarcinoma

Next, we investigated the impact of locoregional and systemic delivery of CDH17 CAR-T cells in a xenograft mouse model of pancreatic adenocarcinoma. In this model, the AsPC-1 PDAC cell line was injected intrapancreas into immunodeficient NSG mice, which were treated with CDH17 CAR-T cells either intrapancreas or intravenous. Both IgG1 hinge- and NWL-spaced CAR-T cells showed antitumor activity compared to control untransduced T cells, regardless of whether they were administered locally or systemically (FIG. 23A). However, CDH17 CAR-T cells infused intrapancreas achieved the best survival benefit (FIG. 23B), suggesting that the optimal delivery route should be evaluated on a case-by-case basis depending on the CAR specificity and tumor settings, as performance and trafficking of CAR-T cells may be influenced by inherent structural characteristics that can differ between distinct tumor types.

### Example 14: Generation and In Vitro Efficacy of Third Generation CDH17 CAR-T Cells

Third generation CARs are generated by combining two or more co-stimulatory domains, most frequently CD28 and 4-1BB, in the CAR intracellular portion. Since CD28 and 4-1BB activate distinct signaling pathways with different downstream effects on T cells, third generation CARs exploit the synergistic features of the two co-stimuli to enhance CAR-T cells fitness and efficacy. In several reported studies, third generation CARs demonstrated stronger cytokine release and tumor killing in vitro and increased expansion and longer persistence in vivo. Therefore, we decided to generate third generation CDH17.28BBz CAR-T cells to test if they confer an advantage compared to second generation CDH17.28z CAR-T cells. The CDH17.28BBz A4_4R CAR was generated by joining the A4_4R scFv, the IgG4 hinge spacer, the CD28 transmembrane domain and an intracellular domain consisting of both CD28 and 4-1BB co-stimuli, and CD32. We produced a third generation CAR construct developed by Feng et al. (Feng et al. (2022) Nat Cancer) with the idea of comparing the impact of our A4_4R scFv versus their antigen-recognition domain on CARs efficacy, which comprises a llama-derived VHH1 nanobody (CDH17.28BBZ VHH1). All CAR constructs were then cloned into bidirectional lentiviral vectors including the NGFR selection marker in antisense orientation and the CAR gene downstream the PGK promoter (FIG. 24A). Primary human T lymphocytes from healthy donors were stimulated with anti-CD3/CD28 beads, transduced with the described LV vectors and expanded with interleukin-7 (IL-7) and IL-15 (FIG. 24B). At the end of manufacturing, all CAR-T cells generated showed high and similar transduction efficiencies and a great representation of both CD4 and CD8 (FIGS. 24C-E). Upon co-culture with LoVo tumors cells, second generation CDH17.28z CAR-T cells showed the best antitumor killing (FIGS. 24 F and G) and the highest production of inflammatory cytokines (FIG. 24H). Focusing on third generation CARs carrying different antigen-recognition domains, A4_4R CAR T cells showed higher tumor killing than VHH1 CAR T cells at low E:T ratios (FIGS. 24 F and I).

CDH17.28BBz A4_4R CAR-T cells were successfully manufactured from patients suffering from CRC-LM, as they displayed a good expansion kinetic upon stimulation with anti-CD3/CD28 beads and interleukin-7 (IL-7) and IL-15, a high transduction efficiency and a good representation of CD4 and CD8 T cells as well as memory T cell subsets (FIGS. 25A-D).

### Additional Material and Methods (Examples 4 to 14)

### Cells and Culture Conditions

T cells were derived from peripheral blood mononuclear cells of healthy donors and of patients with liver metastasis from colorectal carcinoma, pancreatic ductal adenocarcinoma or primary pancreatic ductal adenocarcinoma. All procedures were approved by the Institutional Review Board of IRCCS San Raffaele Scientific Institute and were compliant with all relevant ethical regulations. Patient samples were collected at the Clinical Department at Ospedale San Raffaele (Milan, Italy) under written informed consent in agreement with the Declaration of Helsinki (Protocol “LiMet”, Milan, Italy). Samples obtained for routine diagnostic or monitoring purposes were processed and stored by the institutional biobank Biological Resource Center (CRB-OSR) (Num ID CRB in BBMRI-ERIC: bbmri-eric: ID: IT_1383758011993577). T cells were activated with CD3/CD28 beads (Gibco, 40203D) at 3:1 ratio, transduced at day 2 and cultured in RPMI-1640 (Euroclone, ECB90062L) supplemented with penicillin/streptomycin (100 UI/ml; Lonza, DE17-602E), glutamine (2 mM; Lonza, LOBE17-605E), 10% FBS (fetal bovine serum, Carlo Erba, FA30WS1810500) and interleukin IL-7 and IL-15 (5 ng/ml, Peprotech, 200-07, 200-15). At day 6, beads were removed and CAR T cells were expanded in complete medium, as previously described (Casucci et al. (2013) Blood; Casucci et al. (2018) Front Immunol). Phenotypic analysis and functional testing were performed at the end of manufacturing. Human colorectal cancer cell lines (LoVo and SW620) and human pancreatic cancer cell line (AsPC-1) were cultured with RPMI-1640 (Euroclone) supplemented with penicillin/streptomycin (100 UI/ml; Lonza, DE17-602E), glutamine (2 mM; Lonza, LOBE17-605E) and 10% FBS (fetal bovine serum, Carlo Erba, FA30WS1810500). Human HEK-293T cells were used as packaging line for lentiviral production and were cultured in Iscove's Modified Dulbecco's Medium (IMDM, Euroclone, ECB2072L). For in vivo studies, tumor cells were transduced with a secreted luciferase to allow tumor growth monitoring. All cells were routinely tested for mycoplasma contamination by PCR (Mycoplasmacheck, Eurofins Genomics) and proved negative.

### Generation of CAR Constructs

Second generation CEA.28z CAR construct contains scFv derived from BW431-26 mAb. CAR incorporates an IgG1-derived hinge spacer, a CD28 trans-membrane and costimulatory domain, and a CD3ξ endodomain. Third generation CDH17.28BBz_A4_4R and CDH17.28BBz_VHH1 CAR constructs were generated by cloning CAR-encoding sequences into bidirectional LV carrying the human PGK strong promoter and mCMV to enhance upstream transcription. For both these constructs, ΔLNGFR selection marker was placed in antisense orientation, while CAR-encoding sequences were cloned immediately downstream to the PGK promoter. CDH17.28BBz_A4_4R CAR construct contains A4_4R scFv, IgG4 hinge, CD28 transmembrane domain, CD28 and 4-1BB co-stimulatory domain, and CD3ξ endodomain. CDH17.28BBz_VHH1 CAR construct contains VHH1 nanobody (Feng et al. (2022) Nature Cancer), IgG4 hinge, CD28 transmembrane domain, CD28 and 4-1BB co-stimulatory domain and CD3ξ endodomain.

Generation of CDH17 Deleted Mutants and Identification of A4 4R scFv Binding Site

CDH17 deletion mutants (ΔEC1-ΔEC7) were generated by cloning CDH17 complementary DNA sequences with individual EC domain deletion synthetized by Twist Bioscience and by GeneArt (Thermo Fisher Scientific) into bidirectional lentiviral vectors carrying the human PGK strong promoter and mCMV to enhance upstream transcription. In this platform, ΔNGFR selection marker was placed in antisense orientation, while CDH17 mutant-encoding sequences were cloned immediately downstream to the PGK promoter. For determining the binding site of CDH17 by A4_4R scFv, full-length CDH17 or CDH17 deletion mutants were transduced into CDH17^{neg}SW620 colorectal cancer cells. The transduced cells were stained with anti-human fluorophore-conjugated antibodies specific for CDH17 FITC (Santa Cruz, Clone H1-1) and nerve growth factor receptor (NGFR) PE-Cy7 (clone C40-1457, BD Biosciences, 562122). The transduced cells were co-cultured with CDH17.28z cells or with control untransduced T cells. Elimination index was calculated as follows: 1−(number of residual target cells with experimental CAR T cells/number of residual target cells with control T cells). SW620 wild type cells were used as negative control.

### Sequencing

To verify that CDH17 deletion mutants cDNA inserts were correctly cloned into the lentiviral backbone, DNA sequencing was performed using forward primer 5′-GACCGAATCACCGACCTCTCT-3′ and reverse primer 5′-AATCCAGAGGTTGATT GTCGA-3′ (Eurofins Genomics).

### LV Vector Production and Titration

To produce LV supernatants, 293T cells were co-transfected with the transfer vector, the packaging plasmid (pMDLg/pRRE, encoding for viral genes gag-pol; Addgene, Cambridge, MA), the REV plasmid (pRSV-Rev, encoding for the viral gene rev; Addgene) and the ENV plasmid (pMD2.VSV-G, encoding for G glycoprotein of the vesicular stomatitis virus pericapsis-VSV-G-; Addgene), using CaCl₂) precipitation. Supernatants containing lentiviruses were collected 48 hours later, ultracentrifuged and cryopreserved. To titrate LV supernatants, 293T cells were transduced with different supernatant dilutions. After 6 days from the titration, 293T cells were analyzed by FACS and the titer of LV supernatants was calculated.

Generation of Patient Derived Organoids (PDOs) from CRC-Liver Metastases Samples

Tissue resections were obtained from patients undergoing surgery at IRCCS San Raffaele Hospital, for the removal of clinically confirmed liver metastases from colorectal cancer. Human samples were obtained after written informed consent and IRB approval (Protocol “Limet”, Milan, Italy). Tissue samples were kept in cold in Phosphate Buffered Saline (PBS) (Euroclone) until processing and for no more than 24 hours after surgery resection. Tissues were processed, by cutting them with scalpels in small pieces and incubating them with PBS+5 mM EDTA (Invitrogen). Tissues fragments were washed in PBS and then incubated for 1 hour at room temperature with a digestion solution, composed by PBS/EDTA 1 mM+TrypLE 10× (Gibco)+10× DNAse I buffer+DNAsel (Roche). Dissociated cells were collected in Advanced DMEM/F12 medium (Gibco), pelleted and resuspended in 130 μl of Growth Factor Reduced (GFR) Matrigel matrix (Corning) and seeded in single 24 mws plate. After Matrigel solidification, complete human organoid medium was added to the plate. Basal medium was prepared using the following reagents: Advanced DMEM F/12 (Gibco) with the addition of 1% penicillin/streptomycin (100 U/ml, 0.1 mg/ml, Euroclone) and 1% Glutamine (2 mM, Euroclone), 1×B-27 (Gibco), 1×N-2 (Gibco) and 0.1% BSA. Basal medium was complemented with the following additives: EGF (PrepoTech), Noggin (PrepoTech), RSpondin-1 (PrepoTech), Gastrin (Sigma-Aldrich), FGF-10 (PrepoTech), FGF-basic (PrepoTech), WNT-3A (R&D Systems), Prostaglandin E2 (Tocris), Y-27632 (Stem Cell Thecnologies), Nicotinamide (Sigma-Aldrich), A 83-01 (Tocris), SB202190 (SigmaAldrich), HGF (PrepoTech). Additive solutions were prepared at 1000× concentration and freshly added to the basal medium. To perform in vitro functional assays, PDOs were transduced with Luciferase-expressing lentiviral vector and selected in puromycin. For in vitro functional assays, PDOs were harvested by gently pipetted out of Matrigel using 1 ml Cell Recovery Solution (Corning) per well and incubated for 45 minutes at 4° C. After the incubation, PDOs were collected in Falcon tubes, diluted five times in HBSS and centrifuged. Supernatant was discarded and the pellet was resuspended in a 1:1 ratio Matrigel and basal medium.

### In Vitro Functional Assays

CAR T cells or control untransduced T cells were co-cultured with target cells at different E:T ratios in RPMI-1640 fully supplemented in the absence of cytokines. After 24 hours, supernatants were collected and analyzed with LEGENDplex™ bead-based cytokine immunoassay (BioLegend, 740725). After 4 days (tumors) surviving cells were counted using Flow-Count Fluorospheres (Beckman Coulter, 7547053) and analysed by flow cytometry. Elimination index was calculated as follows: 1−(number of residual target cells with experimental CAR T cells/number of residual target cells with control T cells). In co-culture assays addressing the killing of PDOs after 3 days, surviving organoids were quantified by bioluminescence assay using Tristar 3 filter-based multimode plate reader (Berthold) and representative bright-field microscopy images were taken. Residual tumor level was expressed as relative light unit (RLU). Elimination index was calculated as follows: 1−(RLU of residual target cells with experimental CAR T cells/RLU of residual target cells with control T cells).

### Mouse Experiments

All experiments were approved by the Institutional Animal Care and Use Committee of IRCCS San Raffaele Scientific Institute and by the Italian Governmental Health Institute. Female or male 6- to 9-week-old NOD.Cg-Prkdcscid Il2rgtm1Wjl (NSG) mice (Charles River Laboratories) and NSGTgCMV-IL3, CSF2, KITLG1Eav/MloySzJ (SGM3) mice (Charles River Laboratories) were kept in a specific pathogen-free facility within individually ventilated cages. For experiments addressing anti-tumor potency of CDH17.28z cells 1.5×10⁶ LoVo LUCIA+NGFR+ cells and 4×10⁶ AsPC-1 LUCIA+NGFR+ cells were subcutaneously injected and 10×10⁶ CAR T cells were administered intravenously. For experiments validating the administration route of treatment, 0.1×10⁶ LoVo LUCIA+NGFR+ cells were intrahepatic infused and 1.5×10⁶ AsPC-1 LUCIA+NGFR+ cells were intrapancreatic administered. 10×10⁶ CAR T cells were delivered intrahepatic and intrapancreatic, respectively. For experiments assessing the potential systemic toxicity of CDH17.28z cells, SGM3 mice were sublethally irradiated and infused intravenous with 1×10⁵ human cord blood (CB) derived hematopoietic stem and progenitor cells (HSPCs) CD34+ purified from umbilical CB samples collected at the Gynecology Unit at IRCCS Ospedale San Raffaele (Milan, Italy) under written informed consent approved by IRCCS Ospedale San Raffaele Ethics Committee (Protocol 34CB, Milan, Italy). Upon reconstitution, HSPC-humanized SGM3 mice were infused intrahepatic with 0.1×10⁶ LoVo LUCIA+NGFR+ cells and treated with 5×10⁶ CAR T cells delivered either intrahepatic or intravenous. For evaluating cytokine release syndrome development, weight loss was daily monitored, and the concentration of serum human cytokines was assessed at day 10 with LEGENDplex™ bead-based cytokine immunoassay (BioLegend, 740724). In all experiments, tumor growth was monitored by bioluminescence assay using the QUANTI-Luc detection reagent (InvivoGen, rep-qlc1) and expressed as relative light units (RLUs). Mice were euthanized at RLU≥10⁵ in control groups. At euthanasia, subcutaneous tumor masses were retrieved, dissociated using gentleMACS (Miltenyi Biotec, 130-093-235) and tumor dissociation reagents (Miltenyi Biotec, 130-095-929), and analyzed by flow cytometry.

### Flow Cytometry

Samples were washed with phosphate-buffered saline (PBS) containing 5% fetal bovine serum (FBS) and stained at 4° C. for 20 minutes. Prior to use, all antibodies were validated and titrated for the optimal on target/off target activity on human peripheral blood cells or tumor cell lines. We used mouse anti-human fluorophore-conjugated antibodies specific for: CD3 allophycocyanin (APC)-Cy7 (clone SK7, BioLegend, 344818), CD45 BV510 (clone 30-F11, BioLegend, 103137), CD8 PerCP (clone SK1, BD Biosciences, 345774), CD4 PB (clone OKT4, BioLegend, 317423), CD45RA fluorescein isothiocyanate (FITC, clone HI100, BioLegend, 983002), CD62L APC (clone DREG-56, BioLegend, 304810), nerve growth factor receptor (NGFR) PE (clone C40-1457, BD Biosciences, 557196), CD20 PE (clone 2H7, BioLegend, 980214), CDH17 FITC (clone H1-1, Santa Cruz, sc-393533), major histocompatibility complex II receptor (HLA-DR) APC-Cy7 (clone L243, BioLegend, 307618). 4′,6-diamidino-2-phenylindole (DAPI) was used to determine cell vitality. CAR transduction efficiency was determined by staining with an anti-CD20 monoclonal antibody or with an anti-NGFR monoclonal antibody reactive toward antisense gene marker. Relative Fluorescent Intensity (RFI) was calculated as the ratio of the mean fluorescence intensities (MFI) of a specific fluorophore-conjugated antibody over a fluorophore-conjugated control. Either secondary antibodies or control isotypes were used as control. Data were collected using FACS Canto II (BD Biosciences) or CytoFLEX (Beckman Coulter) flow cytometers. Data were analyzed with FlowJo Software.

### Immunohistochemical and Immunofluorescence Analysis

For IHC, tumor samples were incubated with commercially available anti-CDH17 antibody (clone EPR3996, Abcam, ab109190) according to manufacturer's instructions. Briefly, tumor sections were deparaffinized and heat mediated antigen retrieval was performed before proceeding with IHC staining protocol. For IF, healthy and tumor samples were incubated with indicated primary antibodies, according to manufacturer's instructions. Confocal images were acquired using Leica TCS SP8 confocal microscope (Leica Microsystems) with an HC PLAPO CS 2 63× (NA 1.4) oil objective equipped with a white light laser (470-670 nm) as z stacks. Digital images were recorded in separately scanned channels with no overlap in detection of emissions from the respective fluorochromes. Final image processing was performed with ImageJ Software with minimal contrast and luminosity adjustment.

### Statistical Analysis

Statistical analysis was performed on GraphPad Prism 10.0.2 software and are presented as mean+/−SEM as stated in the figure legends. Datasets were analyzed with paired or unpaired Student's t test, one-way or two-way analysis of variance (ANOVA), and the log-rank Mantel-Cox tests, depending on the experimental design. Appropriate statistical tests were used as described in the figure legends. Differences with a P value<0.05 were considered statistically significant.

### Embodiments

Various preferred features and embodiments of the present invention will now be described with reference to the following numbered paragraphs (paras).
1. A chimeric antigen receptor (CAR) comprising an antigen-binding domain, wherein the antigen-binding domain comprises:
   a) heavy chain variable region (VH) complementarity determining regions (CDRs) with the sequences: CDR1—DHTIHWMR (SEQ ID NO: 1); CDR2—YIYPRDGITGYNERFRGK (SEQ ID NO: 2); and CDR3—WGYSYRNYAYYYDYWGQGTL (SEQ ID NO: 3); or variants thereof each having up to three amino acid substitutions, additions or deletions; and
   b) light chain variable region (VL) CDRs with the sequences: CDR1—INCRSSQSLLHSSNQR (SEQ ID NO: 4); CDR2—PPKVLIYWASTRES (SEQ ID NO: 5); and CDR3—QQYYSYPWTFGQ (SEQ ID NO: 6); or variants thereof each having up to three amino acid substitutions, additions or deletions.
2. The CAR of para 1, wherein the antigen-binding domain comprises:
   a) a VH domain comprising the sequence of SEQ ID NO: 7; and
   b) a VL domain comprising the sequence of SEQ ID NO: 8;
   or variants thereof, each having at least 75% sequence identity thereto.
3. The CAR of para 1 or para 2, wherein the antigen-binding domain comprises a single-chain variable fragment (scFv).
4. The CAR of any one of paras 1 to 3, wherein the antigen-binding domain comprises a sequence having at least 75% sequence identity to the sequence of SEQ ID NO: 9.
5. The CAR of any one of paras 1 to 4, wherein the CAR comprises:
   a) a CD28, a CD8, and/or a CD4 transmembrane domain;
   b) an IgG1 hinge, LNGFR spacer or mCH2CH3 spacer;
   c) a CD28 and/or a 4-1BB co-stimulatory domain; and/or
   d) a CD3-zeta signalling domain.
6. The CAR of any one of paras 1 to 5, wherein the CAR comprises a IgG1 hinge that comprises a sequence with at least 75% sequence identity to the sequence of SEQ ID NO: 10.
7. The CAR of any one of paras 1 to 5, wherein the CAR comprises a LNGFR spacer that comprises a sequence having at least 75% sequence identity to the sequence of any one of SEQ ID NOs: 11, 27, 32, or 33.
8. The CAR of any one of paras 1 to 5, wherein the CAR comprises a mCH2CH3 spacer that comprises a sequence having at least 75% sequence identity to the sequence of SEQ ID NO: 28.
9. The CAR of any one of paras 1 to 8, wherein the CAR comprises a CD3-zeta signalling domain that comprises a sequence having at least 75% sequence identity to the sequence of SEQ ID NO: 13.
10 The CAR of any one of paras 1 to 9, wherein the CAR comprises a sequence having at least 75% sequence identity to the sequence of any one of SEQ ID NOs: 9, 14, 15, 23, 24, or 47.
11. A single-chain variable fragment (scFv) comprising:
   a) heavy chain variable region (VH) complementarity determining regions (CDRs) with the sequences: CDR1—DHTIHWMR (SEQ ID NO: 1); CDR2—YIYPRDGITGYNERFRGK (SEQ ID NO: 2); and CDR3—WGYSYRNYAYYYDYWGQGTL (SEQ ID NO: 3); or variants thereof each having up to three amino acid substitutions, additions or deletions; and
   b) light chain variable region (VL) CDRs with the sequences: CDR1—INCRSSQSLLHSSNQR (SEQ ID NO: 4); CDR2—PPKVLIYWASTRES (SEQ ID NO: 5); and CDR3—QQYYSYPWTFGQ (SEQ ID NO: 6); or variants thereof each having up to three amino acid substitutions, additions or deletions.
12. The scFv of para 11, wherein the scFv comprises a sequence having at least 75% sequence identity to the sequence of SEQ ID NO: 9.
13. A polynucleotide comprising one or more nucleotide sequences encoding the CAR of any one of paras 1 to 10, or the scFv of para 11 or para 12.
14. The polynucleotide of para 13, wherein the polynucleotide comprises:
   a) a sequence encoding the scFv that comprises the sequence of SEQ ID NO: 16; and/or
   b) a sequence encoding the CAR that comprises the sequence of any one of SEQ ID NOs: 16-18, 25-26, 51 or 85;
   or variants thereof, each having at least 75% sequence identity thereto.
15. A vector comprising the polynucleotide of para 13 or para 14.
16. A cell comprising the CAR of any one of paras 1 to 10, the scFv of para 11 or para 12, the polynucleotide of para 13 or para 14, or the vector of para 15.
17. The cell of para 16, wherein the cell is selected from the group consisting a of T cell, Natural Killer (NK) cell, invariant-NK T cell, Cytokine-Induced Killer (CIK) cell, and macrophage, optionally wherein the cell is an autologous or allogeneic cell.
18. A pharmaceutical composition comprising the CAR of any one of paras 1 to 10, the scFv of para 11 or para 12, the polynucleotide of para 13 or para 14, the vector of para 15, or the cell of para 16 or para 17.
19 The CAR of any one of paras 1 to 10, the scFv of para 11 or para 12, the polynucleotide of para 13 or para 14, the vector of para 15, the cell of para 16 or para 17, or the pharmaceutical composition of para 18 for use in therapy.
20 The CAR, the scFv, the polynucleotide, the vector, the cell, or the pharmaceutical composition for use according to para 19, wherein the therapy is treatment of cancer.
21 The CAR, the scFv, the polynucleotide, the vector, the cell, or the pharmaceutical composition for use according to para 19 or para 20, wherein:
   a) the cancer is primary cancer, optionally a gastrointestinal cancer, a colorectal cancer, a pancreatic cancer and/or a stomach cancer;
   b) the cancer is a secondary cancer, optionally a liver metastasis, optionally a liver metastasis of a colorectal cancer, and/or a liver metastasis of a pancreatic ductal adenocarcinoma (PDAC); and/or
   c) the cancer is a neuroendocrine tumor.
22. Use of the scFv of para 11 or para 12, for determining the level of cadherin-17 (CDH-17) in a sample, optionally wherein the sample is from a subject.
23. A method for identifying a subject suitable for treatment with an anti-CDH-17 therapy, wherein the method comprises determining a CDH-17 expression level in a sample isolated from the subject, wherein the CDH-17 expression level is determined using the scFv of para 11 or para 12.
24. A chimeric antigen receptor (CAR) comprising an antigen-binding domain, wherein the antigen-binding domain comprises:
   a) heavy chain variable region (VH) complementarity determining regions (CDRs) with the sequences: CDR1—DYYMY (SEQ ID NO: 42); CDR2—SISFDGTYTYYTDRVKG (SEQ ID NO: 43); and CDR3—DRPAWFPY (SEQ ID NO: 44); or variants thereof each having up to three amino acid substitutions, additions or deletions; and
   b) light chain variable region (VL) CDRs with the sequences: CDR1—RSSQSIVHSNGNTYLE (SEQ ID NO: 39); CDR2—KVSNRFS (SEQ ID NO: 40); and CDR3—FQGSHVPLT (SEQ ID NO: 41); or variants thereof each having up to three amino acid substitutions, additions or deletions.
25 The CAR of para 24, wherein the antigen-binding domain comprises:
   a) a VH domain comprising the sequence of SEQ ID NO: 58; and
   b) a VL domain comprising the sequence of SEQ ID NO: 59;
   or variants thereof, each having at least 75% sequence identity thereto.
26 The CAR of para 24 or para 25, wherein the antigen-binding domain comprises a single-chain variable fragment (scFv).
27 The CAR of any one of paras 24 to 26, wherein the antigen-binding domain comprises a sequence having at least 75% sequence identity to the sequence of SEQ ID NO: 45.
28. The CAR of any one of paras 24 to 27, wherein the CAR comprises:
   a) a CD28, a CD8, and/or a CD4 transmembrane domain;
   b) an IgG1 hinge, LNGFR spacer or mCH2CH3 spacer;
   c) a CD28 and/or a 4-1BB co-stimulatory domain; and/or
   d) a CD3-zeta signalling domain.
29. The CAR of any one of paras 24 to 28, wherein the CAR comprises a IgG1 hinge that comprises a sequence with at least 75% sequence identity to the sequence of SEQ ID NO: 10.
30. The CAR of any one of paras 24 to 28, wherein the CAR comprises a LNGFR spacer that comprises a sequence having at least 75% sequence identity to the sequence of any one of SEQ ID NOs: 11, 27, 32, or 33.
31. The CAR of any one of paras 24 to 28, wherein the CAR comprises a mCH2CH3 spacer that comprises a sequence having at least 75% sequence identity to the sequence of SEQ ID NO: 28.
32. The CAR of any one of paras 24 to 31, wherein the CD3-zeta signalling domain comprises a sequence having at least 75% sequence identity to the sequence of SEQ ID NO: 13.
33 The CAR of any one of paras 24 to 32, wherein the CAR comprises a sequence having at least 75% sequence identity to the sequence of any one of SEQ ID NOs: 48-50.
34. A single-chain variable fragment (scFv) comprising:
   a) heavy chain variable region (VH) complementarity determining regions (CDRs) with the sequences: CDR1—DYYMY (SEQ ID NO: 42); CDR2—SISFDGTYTYYTDRVKG (SEQ ID NO: 43); and CDR3—DRPAWFPY (SEQ ID NO: 44); or variants thereof each having up to three amino acid substitutions, additions or deletions; and
   b) light chain variable region (VL) CDRs with the sequences: CDR1—RSSQSIVHSNGNTYLE (SEQ ID NO: 39); CDR2—KVSNRFS (SEQ ID NO: 40); and CDR3—FQGSHVPLT (SEQ ID NO: 41); or variants thereof each having up to three amino acid substitutions, additions or deletions.
35. The scFv of para 34, wherein the scFv comprises a sequence having at least 75% sequence identity to the sequence of SEQ ID NO: 45.
36. A polynucleotide comprising one or more nucleotide sequences encoding the CAR of any one of paras 24 to 33, or the scFv of para 34 or para 35.
37 The polynucleotide of para 36, wherein the polynucleotide comprises:
   a) a sequence encoding the scFv that comprises the sequence of SEQ ID NO: 46; and/or
   b) a sequence encoding the CAR that comprises the sequence of any one of SEQ ID NOs: 46, and/or 52-54;
   or variants thereof, each having at least 75% sequence identity thereto.
38. A vector comprising the polynucleotide of para 36 or para 37.
39. A cell comprising the CAR of any one of paras 24 to 33, the scFv of para 34 or para 35, the polynucleotide of para 36 or para 37, or the vector of para 38.
40. The cell of para 39, wherein the cell is selected from the group consisting a of T cell, Natural Killer (NK) cell, invariant-NK T cell, Cytokine-Induced Killer (CIK) cell, and macrophage, optionally wherein the cell is an autologous or allogeneic cell.
41. A pharmaceutical composition comprising the CAR of any one of paras 24 to 33, the scFv of para 34 or para 35, the polynucleotide of para 36 or para 37, the vector of para 38, or the cell of para 39 or para 40.
42. The CAR of any one of paras 24 to 33, the scFv of para 34 or para 35, the polynucleotide of para 36 or para 37, the vector of para 38, the cell of para 39 or para 40, or the pharmaceutical composition of para 41 for use in therapy.
43. The CAR, the scFv, the polynucleotide, the vector, the cell, or the pharmaceutical composition for use according to para 42, wherein the therapy is treatment of cancer.
44. The CAR, the scFv, the polynucleotide, the vector, the cell, or the pharmaceutical composition for use according to para 42 or para 43, wherein:
   a) the cancer is primary cancer, optionally a gastrointestinal cancer, a colorectal cancer, a pancreatic cancer and/or a stomach cancer;
   b) the cancer is a secondary cancer, optionally a liver metastasis, optionally a liver metastasis of a colorectal cancer, and/or a liver metastasis of a pancreatic ductal adenocarcinoma (PDAC); and/or
   c) the cancer is a neuroendocrine tumor.
45 Use of the scFv of para 34 or para 35, for determining the level of cadherin-17 (CDH-17) in a sample, optionally wherein the sample is from a subject.
46. A method for identifying a subject suitable for treatment with an anti-CDH-17 therapy, wherein the method comprises determining a CDH-17 expression level in a sample isolated from the subject, wherein the CDH-17 expression level is determined using the scFv of para 34 or para 35.

1. A chimeric antigen receptor (CAR) comprising an antigen-binding domain, wherein the antigen-binding domain comprises:
(i) a) heavy chain variable region (VH) complementarity determining regions (CDRs) with the sequences: CDR1—DHTIHWMR (SEQ ID NO: 1); CDR2—YIYPRDGITGYNERFRGK (SEQ ID NO: 2); and CDR3—WGYSYRNYAYYYDYWGQGTL (SEQ ID NO: 3); or variants thereof each having up to three amino acid substitutions, additions or deletions; and b) light chain variable region (VL) CDRs with the sequences: CDR1—INCRSSQSLLHSSNQR (SEQ ID NO: 4); CDR2—PPKVLIYWASTRES (SEQ ID NO: 5); and CDR3—QQYYSYPWTFGQ (SEQ ID NO: 6); or variants thereof each having up to three amino acid substitutions, additions or deletions; or
(ii) a) heavy chain variable region (VH) complementarity determining regions (CDRs) with the sequences: CDR1—DYYMY (SEQ ID NO: 42); CDR2—SISFDGTYTYYTDRVKG (SEQ ID NO: 43); and CDR3—DRPAWFPY (SEQ ID NO: 44); or variants thereof each having up to three amino acid substitutions, additions or deletions; and b) light chain variable region (VL) CDRs with the sequences: CDR1—RSSQSIVHSNGNTYLE (SEQ ID NO: 39); CDR2—KVSNRFS (SEQ ID NO: 40); and CDR3—FQGSHVPLT (SEQ ID NO: 41); or variants thereof each having up to three amino acid substitutions, additions or deletions.

2. The CAR of claim 1, wherein the antigen-binding domain comprises:
a) a VH domain comprising the sequence of SEQ ID NO: 7 and a VL domain comprising the sequence of SEQ ID NO: 8, or variants thereof, each having at least 75% sequence identity thereto; or
b) a VH domain comprising the sequence of SEQ ID NO: 58 and a VL domain comprising the sequence of SEQ ID NO: 59, or variants thereof, each having at least 75% sequence identity thereto.

3. The CAR of claim 1 or claim 2, wherein the antigen-binding domain comprises a single-chain variable fragment (scFv), optionally wherein the antigen-binding domain comprises a sequence having at least 75% sequence identity to the sequence of SEQ ID NO: 9 or SEQ ID NO: 45.

4. The CAR of any one of claims 1 to 3, wherein the CAR comprises:
a) a CD28, a CD8, and/or a CD4 transmembrane domain;
b) an IgG1 hinge, LNGFR spacer or mCH2CH3 spacer;
c) a CD28 and/or a 4-1BB co-stimulatory domain; and/or
d) a CD3-zeta signalling domain;
optionally wherein:
(i) the CAR comprises a CD28 transmembrane domain that comprises a sequence having at least 75% sequence identity to the sequence of SEQ ID NO: 61;
(ii) the CAR comprises a IgG1 hinge that comprises a sequence with at least 75% sequence identity to the sequence of SEQ ID NO: 10;
(iii) the CAR comprises a LNGFR spacer that comprises a sequence having at least 75% sequence identity to the sequence of any one of SEQ ID NOs: 11, 27, 32, or 33;
(iv) the CAR comprises a mCH2CH3 spacer that comprises a sequence having at least 75% sequence identity to the sequence of SEQ ID NO: 28; and/or
(v) the CAR comprises a CD3-zeta signalling domain that comprises a sequence having at least 75% sequence identity to the sequence of SEQ ID NO: 13.

5. The CAR of any one of claims 1 to 4, wherein the CAR comprises a sequence having at least 75% sequence identity to the sequence of any one of SEQ ID NOs: 9, 14, 15, 23, 24, 47, 48, 49, or 50.

6. A single-chain variable fragment (scFv) comprising:
(i) a) heavy chain variable region (VH) complementarity determining regions (CDRs) with the sequences: CDR1—DHTIHWMR (SEQ ID NO: 1); CDR2—YIYPROGITGYNERFRGK (SEQ ID NO: 2); and CDR3—WGYSYRNYAYYYDYWGQGTL (SEQ ID NO: 3); or variants thereof each having up to three amino acid substitutions, additions or deletions; and b) light chain variable region (VL) CDRs with the sequences: CDR1—INCRSSQSLLHSSNQR (SEQ ID NO: 4); CDR2—PPKVLIYWASTRES (SEQ ID NO: 5); and CDR3—QQYYSYPWTFGQ (SEQ ID NO: 6); or variants thereof each having up to three amino acid substitutions, additions or deletions; or
(ii) a) heavy chain variable region (VH) complementarity determining regions (CDRs) with the sequences: CDR1—DYYMY (SEQ ID NO: 42); CDR2—SISFDGTYTYYTDRVKG (SEQ ID NO: 43); and CDR3—DRPAWFPY (SEQ ID NO: 44); or variants thereof each having up to three amino acid substitutions, additions or deletions; and b) light chain variable region (VL) CDRs with the sequences: CDR1—RSSQSIVHSNGNTYLE (SEQ ID NO: 39); CDR2—KVSNRFS (SEQ ID NO: 40); and CDR3—FQGSHVPLT (SEQ ID NO: 41); or variants thereof each having up to three amino acid substitutions, additions or deletions;
optionally, wherein the scFv comprises a sequence having at least 75% sequence identity to the sequence of SEQ ID NO: 9 or SEQ ID NO: 45.

7. A polynucleotide comprising one or more nucleotide sequences encoding the CAR of any one of claims 1 to 5, or the scFv of claim 6.

8. A vector comprising the polynucleotide of claim 7.

9. A cell comprising the CAR of any one of claims 1 to 5, the scFv of claim 6, the polynucleotide of claim 7, or the vector of claim 8; optionally, wherein the cell is selected from the group consisting a of T cell, Natural Killer (NK) cell, invariant-NK T cell, Cytokine-Induced Killer (CIK) cell, and macrophage; further optionally wherein the cell is an autologous or allogeneic cell.

10. A pharmaceutical composition comprising the CAR of any one of claims 1 to 5, the scFv of claim 6, the polynucleotide of claim 7, the vector of claim 8, or the cell of claim 9.

11. The CAR of any one of claims 1 to 5, the scFv of claim 6, the polynucleotide of claim 7, the vector of claim 8, the cell of claim 9, or the pharmaceutical composition of claim 10 for use in therapy.

12. The CAR, the scFv, the polynucleotide, the vector, the cell, or the pharmaceutical composition for use according to claim 11, wherein the therapy is treatment of cancer.

13. The CAR, the scFv, the polynucleotide, the vector, the cell, or the pharmaceutical composition for use according to claim 11 or claim 12, wherein:
a) the cancer is primary cancer, optionally a gastrointestinal cancer, a colorectal cancer, a pancreatic cancer and/or a stomach cancer;
b) the cancer is a secondary cancer, optionally a liver metastasis, optionally a liver metastasis of a colorectal cancer, and/or a liver metastasis of a pancreatic ductal adenocarcinoma (PDAC); and/or
c) the cancer is a neuroendocrine tumor.

14. Use of the scFv of claim 6, for determining the level of cadherin-17 (CDH-17) in a sample, optionally wherein the sample is from a subject.

15. A method for identifying a subject suitable for treatment with an anti-CDH-17 therapy, wherein the method comprises determining a CDH-17 expression level in a sample isolated from the subject, wherein the CDH-17 expression level is determined using the scFv of claim 6.
